(19) United States
(12) Patent Application Publication
(10) Pub. No.: US 2020/0033347 A1
(43) Pub. Date: Jan. 30, 2020

(54) Biomarkers And Targets For Proliferative Diseases

(71) Applicant: Universite Libre de Bruxelles, Brussels (BE)

(72) Inventors: Ahmad Merhi, Brussels (BE); Anna-Maria Marini, Brussels (BE)

(21) Appl. No.: 16/604,444

(22) PCT Filed: Apr. 18, 2018
(86) PCT No.: PCT/EP2018/059907

§ 371 Date: Oct. 10, 2019

(30) Foreign Application Priority Data
Apr. 18, 2017 (EP) 17166834.6

(51) Int. Cl. G01N 33/574; C07K 16/28; C12N 15/113; C12Q 1/6886

(52) U.S. Cl. CPC G01N 33/57415; C07K 16/28; C12N 15/1138; C12Q 2600/106; C07K 2317/34; C12N 2310/14; C12Q 2600/158; C12Q 1/6886

(57) ABSTRACT

The application discloses the use of at least one ammonium transporter as a biomarker and target for proliferative diseases, and related therapeutic or prophylactic agents, kits and methods.

### FIELD OF THE INVENTION

The invention relates to biomarkers and targets for diseases and conditions in subjects, in particular for proliferative diseases, and to related methods, uses, kits and therapeutic or prophylactic agents.

### BACKGROUND

Diseases caused by excessive, uncontrolled cell proliferation, such as various types of cancers, account for a large part of the yearly diagnosed life threatening diseases.

Breast cancer is the most common cancer in women. Due to the complexity and heterogeneity of breast cancer, clinical evolution is difficult to predict and therapeutic and prophylactic treatments are not optimal.

The importance of devising novel and/or improved manners to diagnose and combat proliferative disorders, such as breast cancer, is self-evident. Also important is avoidance of overtreatment in patients who only receive a modest benefit, while suffering from toxic side effects. Individual treatment optimization can be aided by improved methods to distinguish proliferative disease subtypes, such as for breast cancer, luminal and basal breast cancer.

### SUMMARY

The present invention addresses the aforementioned needs, and more particularly teaches novel markers and targets useful for proliferative diseases, such as breast cancer.

As corroborated by the experimental section, which illustrates certain representative embodiments of the invention, the inventors have identified a biological molecule whose quantity or expression level is closely predictive and/or indicative of proliferative diseases, and which thus constitutes a useful and promising biomarker for proliferative diseases. More particularly, the inventors have detected a significantly increased level of Rh family, B glycoprotein (RHBG) in tissue samples obtained from luminal breast tumours when compared to tissue samples obtained from basal breast tumours and/or healthy breast tissues. Therefore, RHBG can be used as a biomarker for proliferative diseases, such as for example as a biomarker for breast cancer or as a biomarker for luminal breast cancer. Furthermore, inhibition of RHBG expression in luminal breast cancer cells decreased proliferation of the cells, corroborating RHBG as a therapeutic target in proliferative diseases, such as for example in breast cancer or in luminal breast cancer.

The inventors also postulate that additional ammonium transporters, such as ammonium transporter belonging to the Mep-Amt-Rh superfamily (Mep-Amt-Rh superfamily includes two families, Mep-Amt and Rh), such as particularly belonging to the Rh family, such as more particularly Rh family, A glycoprotein (RHAG), and Rh family, C glycoprotein (RHCG), can constitute useful biomarkers and therapeutic targets in proliferative diseases, such as for example in breast cancer or in luminal breast cancer.

Accordingly, in an aspect, the invention provides use of at least one ammonium transporter as a biomarker for a proliferative disease in a subject.

In a further aspect, the invention provides a method for the diagnosis, prediction, prognosis and/or monitoring of a proliferative disease in a subject or for determining whether a subject is in need of therapeutic or prophylactic treatment of a proliferative disease, comprising detecting at least one ammonium transporter in a tissue sample from the subject.

In another aspect, the invention provides a kit for diagnosing, predicting, prognosing and/or monitoring a proliferative disease in a subject, the kit comprising:
(i) means for measuring the quantity or expression level of at least one ammonium transporter in a tissue sample from a subject; and
(ii) a reference value of the quantity or expression level of said at least one ammonium transporter or means for establishing said reference value, wherein said reference value represents a known diagnosis, prediction and/or prognosis of the proliferative disease, such as wherein said reference value corresponds to the quantity or expression level of said at least one ammonium transporter in a tissue not affected by the proliferative disease, such as in a healthy tissue, or wherein said reference value corresponds to the quantity or expression level of said at least one ammonium transporter in a tissue affected by the proliferative disease;

preferably wherein said proliferative disease is luminal breast cancer, said tissue sample is obtained from a breast tumour and said reference value represents the quantity or expression level of said at least one ammonium transporter in a healthy breast tissue or in a basal breast cancer tissue

In a further aspect, the invention provides a therapeutic or prophylactic agent capable of modulating, such as inhibiting or increasing, expression or activity of at least one ammonium transporter for use as a medicament. In a further aspect, the invention provides a therapeutic or prophylactic agent capable of modulating, such as inhibiting or increasing, expression or activity of at least one ammonium transporter for use as a medicament in the treatment of a proliferative disease.

In another aspect, the invention relates to an antibody or a functional fragment thereof, characterised in that the antibody or functional fragment thereof binds epitope DSPPRLPALRGPSS of human RHBG as set out in SEQ ID NO: 15.

These and further aspects and preferred embodiments of the invention are described in the following sections and in the appended claims. The subject-matter of the appended claims is hereby specifically incorporated in this specification.

### BRIEF DESCRIPTION OF THE FIGURES

FIG. 1 illustrates RHBG mRNA expression in HMEC (normal), basal and luminal breast cancer cells as determined by quantitative RT-PCR (qRT-PCR).

FIG. 2 illustrates RHBG mRNA expression in formalin-fixed paraffin-embedded (FFPE) basal and luminal breast tumours.

FIG. 3 illustrates immunohistochemistry for RHBG protein expression in formalin-fixed paraffin-embedded (FFPE) basal and luminal breast tumours.

FIG. 4 illustrates that knockdown of RHBG in luminal breast cancer cells decreased cell growth. (A) MCF-7 luminal breast cancer cells were reverse transfected with control or RHBG siRNA for 72 hours. RHBG mRNA expression was determined by qRT-PCR. (B-C) Cell growth was determined by crystal violet staining.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The inventors demonstrated that Rh family, B glycoprotein (RHBG) was (over)expressed in tissue samples from human subjects having a proliferative disease. For example, RHBG level was increased in tissue samples obtained from luminal breast tumour when compared to a tissue samples obtained from basal breast tumour and/or healthy breast tissue. Therefore, RHBG can be used as a biomarker for proliferative diseases, such as for example as a biomarker for breast cancer or as a biomarker for luminal breast cancer. For example, RHBG can be used to identify subjects having or not having luminal breast cancer. Hereby, patients most likely respond to a given therapy can be selected. Furthermore, the inventors demonstrated that inhibition of RHBG in luminal breast cancer cells decreased their proliferation, and thus identified RHBG as a valuable therapeutic target in proliferative diseases, such as for example in breast cancer or in luminal breast cancer.

The inventors also postulate that additional ammonium transporters, such as ammonium transporter belonging to the Mep-Amt-Rh superfamily (Mep-Amt-Rh superfamily includes two families, Mep-Amt and Rh), such as particularly belonging to the Rh family, such as more particularly Rh family, A glycoprotein (RHAG), and Rh family, C glycoprotein (RHCG), can constitute useful biomarkers and therapeutic targets in proliferative diseases, such as for example in breast cancer or in luminal breast cancer.

Accordingly, in a first aspect, the invention provides the use of at least one ammonium transporter as a biomarker for a proliferative disease in a subject.

In certain embodiments, the at least one ammonium transporter belongs to the Mep-Amt-Rh superfamily. For example, the at least one ammonium transporter may belong to the Mep-Amt family or to the Rh family. Presently known mammalian ammonium transporters are found in the Rh family. Preferably, the at least one ammonium transporter belongs to the Rh family.

In certain embodiments, said at least one ammonium transporter is selected from the group consisting of Rh family, B glycoprotein (RHBG), Rh family, A glycoprotein (RHAG), Rh family, C glycoprotein (RHCG), and combinations thereof.

The term “biomarker” is widespread in the art and may broadly denote a biological molecule and/or a detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition.

In certain embodiments, a biomarker as taught herein, such as in particular said at least one ammonium transporter, such as preferably at least one ammonium transporter selected from the group consisting of RHBG, RHAG, RHCG, and combinations thereof, such as particularly preferably RHBG, may be peptide-, polypeptide- and/or protein-based, or nucleic acid-based.

The reference to any marker, including any peptide, polypeptide, protein, or nucleic acid, corresponds to the marker, peptide, polypeptide, protein, nucleic acid, commonly known under the respective designations in the art. The terms encompass such markers, peptides, polypeptides, proteins, or nucleic acids of any organism where found, and particularly of animals, preferably warm-blooded animals, more preferably vertebrates, yet more preferably mammals, including humans and non-human mammals, still more preferably of humans. The terms particularly encompass such markers, peptides, polypeptides, proteins, or nucleic acids with a native sequence, i.e., ones of which the primary sequence is the same as that of the markers, peptides, polypeptides, proteins, or nucleic acids found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or post-translational modifications. Any such variants or isoforms of markers, peptides, polypeptides, proteins, or nucleic acids are intended herein. Accordingly, all sequences of markers, peptides, polypeptides, proteins, or nucleic acids found in or derived from nature are considered “native”. The terms encompass the markers, peptides, polypeptides, proteins, or nucleic acids when forming a part of a living organism, organ, tissue or cell, when forming a part of a biological sample, as well as when at least partly isolated from such sources. The terms also encompass markers, peptides, polypeptides, proteins, or nucleic acids when produced by recombinant or synthetic means.

In certain embodiments, markers, peptides, polypeptides, proteins, or nucleic acids may be human, i.e., their primary sequence may be the same as a corresponding primary sequence of or present in a naturally occurring human markers, peptides, polypeptides, proteins, or nucleic acids. Hence, the qualifier “human” in this connection relates to the primary sequence of the respective markers, peptides, polypeptides, proteins, or nucleic acids, rather than to its origin or source. For example, such markers, peptides, polypeptides, proteins, or nucleic acids may be present in or isolated from samples of human subjects or may be obtained by other means (e.g., by recombinant expression, cell-free transcription or translation, or non-biological nucleic acid or peptide synthesis).

Hence, in certain embodiments, a biomarker as taught herein, such as in particular an ammonium transporter, such as preferably at least one ammonium transporter selected from the group consisting of RHBG, RHAG, RHCG, and combinations thereof, such as particularly preferably RHBG, may be a human biomarker, such as in particular human ammonium transporter, such as preferably at least one ammonium transporter selected from the group consisting of human RHBG, human RHAG, human RHCG, and combinations thereof, such as particularly preferably human RHBG.

The present methods, uses, or products may employ measurements of at least one ammonium transporter. The term “ammonium transporter” as used herein is intended to be synonymous with terms such as “ammonia transporter”, “ammonium/ammonia transporter”, “ammonia/ammonium transporter”, “NH₃ transporter”, “NH₄⁺ transporter”, “NH₃/NH₄⁺ transporter”, or “NH₄⁺/NH₃ transporter”, and encompasses any transporter capable of transporting ammonia (NH₃) and/or ammonium (NH₄⁺) across a membrane, such as across a cell membrane and/or across an intracellular membrane, such as particularly but without limitation from the exterior of a cell into the cytosol of the cell, or from the interior of a cell to the extracellular fluid, or from the cytosol of a cell into the interior of a membrane-bound intracellular organelle, or from the interior of a membrane-bound intracellular organelle into the cytosol of the cell. The term also encompasses transporters capable of transporting methylamine and/or methylammonium. By means of an example, ammonium transporters may catalyse the reaction NH4⁺ (out) [symbol] NH4⁺ (in), wherein ‘out’ and ‘in’ denote environments or locations separated by a membrane structure, such as a biological membrane. Particularly intended are ammonium transporters constituted by channel-forming transmembrane polypeptides or proteins.

As mentioned, the at least one ammonium transporter may belong to the Mep-Amt-Rh superfamily, such as to the Mep-Amt family or to the Rh family, more preferably to the Rh family.

In certain embodiments, the ammonium transporter may be RHBG, hence, in certain embodiments, the present methods, uses, or products may employ measurements of RHBG. The term “Rh family, B glycoprotein” or “RHBG” as used herein, refers to the ammonia/ammonium transporter peptide, polypeptide or protein RHBG or the RHBG gene encoding the RHBG peptide, polypeptide or protein. RHBG is a non-erythroid member of the Rhesus protein family and has been predicted to be a transmembrane protein with 12 membrane spanning domains and with both the N- and C-terminus located in the cytoplasm of the cell. RHBG as used herein may typically denote human RHBG, but may also encompass reference to RHBG in non-human animals.

By means of an example, nucleic acid sequence of human RHBG is annotated under NCBI Genbank (http://www.ncbi.nlm.nih.gov/) accession number NM_020407.4. However, this sequence was found to be incorrect and a cytosine should be added after the cytosine at position 1271, thereby creating a new TGA stop codon which starts at position 1324 (Han K. et al. Expression of the ammonia transporter family member, Rh B glycoprotein, in the human kidney. Am J Physiol Renal Physiol. 2013; 304(7):F972-F981).

Accordingly, the human RHBG nucleic acid coding sequence is as set forth herein (SEQ ID NO: 1):

ATGGCCGGGTCTCCTAGCCGCGCCGCGGGCCGGCGACTGCAGCTTCCCC
TGCTGTGCCTCTTCCTCCAGGGCGCCACTGCCGTCCTCTTTGCTGTCTT
TGTCCGCTACAACCACAAAACCGACGCTGCCCTCTGGCACCGGAGCAAC
CACAGTAACGCGGACAATGAATTTTACTTTCGCTACCCAAGCTTCCAGG
ACGTGCATGCCATGGTCTTCGTGGGCTTTGGCTTCCTCATGGTCTTCCT
GCAGCGTTACGGCTTCAGCAGCGTGGGCTTCACCTTCCTCCTGGCCGCC
TTTGCCCTGCAGTGGTCCACACTGGTCCAGGGCTTTCTCCACTCCTTCC
ACGGTGGCCACATCCATGTTGGCGTGGAGAGCATGATCAATGCTGACTT
TTGTGCGGGGGCCGTGCTCATCTCCTTTGGTGCCGTCCTGGGCAAGACC
GGGCCTACCCAGCTGCTGCTCATGGCCCTGCTGGAGGTGGTGCTGTTTG
GCATCAATGAGTTTGTGCTCCTTCATCTCCTGGGGGTGAGAGATGCCGG
AGGCTCCATGACTATCCACACCTTTGGTGCCTACTTCGGGCTCGTCCTT
TCGCGGGTTCTGTACAGGCCCCAGCTGGAGAAGAGCAAGCACCGCCAGG
GCTCCGTCTACCATTCAGACCTCTTCGCCATGATTGGGACCATCTTCCT
GTGGATCTTCTGGCCTAGCTTCAATGCTGCACTCACAGCGCTGGGGGCT
GGGCAGCATCGGACGGCCCTCAACACATACTACTCCCTGGCTGCCAGCA
CCCTTGGCACCTTTGCCTTGTCAGCCCTTGTAGGGGAAGATGGGAGGCT
TGACATGGTCCACATCCAAAATGCAGCGCTGGCTGGAGGGGTTGTGGTG
GGGACCTCAAGTGAAATGATGCTGACACCCTTTGGGGCTCTGGCAGCTG
GCTTCTTGGCTGGGACTGTCTCCACGCTGGGGTACAAGTTCTTCACGCC
CATCCTTGAATCAAAATTCAAAGTCCAAGACACATGTGGAGTCCACAAC
CTCCATGGGATGCCGGGGGTCCTGGGGGCCCTCCTGGGGGTCCTTGTGG
CTGGACTTGCCACCCATGAAGCTTACGGAGATGGCCTGGAGAGTGTGTT
TCCACTCATAGCCGAGGGCCAGCGCAGTGCCACGTCACAGGCCATGCAC
CAGCTCTTCGGGCTGTTTGTCACACTGATGTTTGCCTCTGTGGGCGGGG
GCCTTGGAGGGCTCCTGCTGAAGCTACCCTTTCTGGACTCCCCCCCCAG
ACTCCCAGCACTACGAGGACCAAGTTCACTGGCAGGTGCCTGGCGAGCA
TGAGGATAAAGCCCAGAGACCTCTGA

Translation of the nucleic acid sequence as set forth in SEQ ID NO 1 provides amino acid sequence of human RHBG protein as annotated under NCBI Genbank accession number Q9H310.2, reproduced herein (SEQ ID NO: 2):

MAGSPSRAAGRRLQLPLLCLFLQGATAVLFAVFVRYNHKTDAALWHRSN
HSNADNEFYFRYPSFQDVHAMVFVGFGFLMVFLQRYGFSSVGFTFLLAA
FALQWSTLVQGFLHSFHGGHIHVGVESMINADFCAGAVLISFGAVLGKT
GPTQLLLMALLEVVLFGINEFVLLHLLGVRDAGGSMTIHTFGAYFGLVL
SRVLYRPQLEKSKHRQGSVYHSDLFAMIGTIFLWIFWPSFNAALTALGA
GQHRTALNTYYSLAASTLGTFALSALVGEDGRLDMVHIQNAALAGGVVV
GTSSEMMLTPFGALAAGFLAGTVSTLGYKFFTPILESKFKVQDTCGVHN
LHGMPGVLGALLGVLVAGLATHEAYGDGLESVFPLIAEGQRSATSQAMH
QLFGLFVTLMFASVGGGLGGLLLKLPFLDSPPRLPALRGPSSLAGAWRA

In certain embodiments, the ammonium transporter may be RHAG, hence, in certain embodiments, the present methods, uses, or products may employ measurements of RHAG. The term “Rh family, A glycoprotein” or “RHAG” as used herein, refers to the ammonia/ammonium transporter peptide, polypeptide or protein RHAG or the RHAG gene encoding the RHAG peptide, polypeptide or protein. RHAG is a non-erythroid member of the Rhesus protein family. RHAG as used herein may typically denote human RHAG, but may also encompass reference to RHAG in non-human animals.

By means of an example, nucleic acid sequence of human RHAG is annotated under NCBI Genbank accession number NM_000324.2. By means of an example, polypeptide sequence of human RHAG is annotated under NCBI Genbank accession number NP_000315.2, reproduced herein (SEQ ID NO: 20):

MRFTFPLMAIVLEIAMIVLFGLFVEYETDQTVLEQLNITKPTDMGIFFE
LYPLFQDVHVMIFVGFGFLMTFLKKYGFSSVGINLLVAALGLQWGTIVQ
GILQSQGQKFNIGIKNMINADFSAATVLISFGAVLGKTSPTQMLIMTIL
EIVFFAHNEYLVSEIFKASDIGASMTIHAFGAYFGLAVAGILYRSGLRK
GHENEESAYYSDLFAMIGTLFLWMFWPSFNSAIAEPGDKQCRAIVNTYF
SLAACVLTAFAFSSLVEHRGKLNMVHIQNATLAGGVAVGTCADMAIHPF
GSMIIGSIAGMVSVLGYKFLTPLFTTKLRIHDTCGVHNLHGLPGVVGGL
AGIVAVAMGASNTSMAMQAAALGSSIGTAVVGGLMTGLILKLPLWGQPS
DQNCYDDSVYWKVPKTR

In certain embodiments, the ammonium transporter may be RHCG, hence, in certain embodiments, the present methods, uses, or products may employ measurements of RHCG. The term “Rh family, C glycoprotein” or “RHCG” as used herein, refers to the ammonia/ammonium transporter peptide, polypeptide or protein RHCG or the RHCG gene encoding the RHCG peptide, polypeptide or protein. RHCG is a non-erythroid member of the Rhesus protein family. RHCG as used herein may typically denote human RHCG, but may also encompass reference to RHCG in non-human animals.

By means of an example, nucleic acid sequences of human RHCG are annotated under NCBI Genbank accession numbers NM_001321041.1 and NM_016321.2. By means of an example, polypeptide sequences of human RHCG are annotated under NCBI Genbank accession numbers NP_001307970.1 and NP_057405.1, reproduced herein (SEQ ID NO: 21):

MAWNTNLRWRLPLTCLLLQVIMVILFGVFVRYDFEADAHWWSERTHKNL
SDMENEFYYRYPSFQDVHVMVFVGFGFLMTFLQRYGFSAVGFNFLLAAF
GIQWALLMQGWFHFLQDRYIVVGVENLINADFCVASVCVAFGAVLGKVS
PIQLLIMTFFQVTLFAVNEFILLNLLKVKDAGGSMTIHTFGAYFGLTVT
RILYRRNLEQSKERQNSVYQSDLFAMIGTLFLWMYWPSFNSAISYHGDS
QHRAAINTYCSLAACVLTSVAISSALHKKGKLDMVHIQNATLAGGVAVG
TAAEMMLMPYGALIIGFVCGIISTLGFVYLTPFLESRLHIQDTCGINNL
HGIPGIIGGIVGAVTAASASLEVYGKEGLVHSFDFQGFNGDWTARTQGK
FQIYGLLVTLAMALMGGIIVGLILRLPFWGQPSDENCFEDAVYWEMPEG
NSTVYIPEDPTFKPSGPSVPSVPMVSPLPMASSVPLVP

Unless otherwise apparent from the context, reference herein to any marker, peptide, polypeptide, protein, or nucleic acid, or fragment thereof may generally also encompass modified forms of said marker, peptide, polypeptide, protein, or nucleic acid, or fragment thereof, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

In certain embodiments, the ammonium transporter may be capable of transporting one or both of ammonia or ammonium across a membrane, but not capable of transporting other substrates or solutes across a membrane. In certain embodiments, the ammonium transporter may be capable of transporting one or more of ammonia, ammonium, methylamine, or methylammonium across a membrane, but not capable of transporting other substrates or solutes across a membrane. In certain embodiments, the ammonium transporter may be capable of transporting one or more of ammonia, ammonium, or CO₂ across a membrane, but not capable of transporting other substrates or solutes across a membrane. In certain embodiments, the ammonium transporter may be capable of transporting one or more of ammonia, ammonium, methylamine, methylammonium, or CO₂ across a membrane, but not capable of transporting other substrates or solutes across a membrane. The reference to such other substrates or solutes in this context does not include the optional cotransport, such as symport or antiport, of a substance such as an ion.

In certain other embodiments, the ammonium transporter may be a protein or polypeptide capable of mediating the membrane transport of one or more of ammonia, ammonium, methylamine, methylammonium, or CO₂, such as particularly one or more of ammonia, ammonium, methylamine, or methylammonium, such as more particularly one or both of ammonia or ammonium, in addition to one or more other substrates or solutes (i.e., substrates or solutes other than an optionally co-transported substance such as an ion).

By means of an example and not limitation, aquaporines have been reported to also transport ammonia (see inter alia Litman et al. Handb Exp Pharmacol. 2009, no. 190: 327-58, “Ammonia and urea permeability of mammalian aquaporins”; Saparov et al. J. Biol. Chem. 2007, vol. 282, 5296-5301, “Fast and selective ammonia transport by aquaporin-8”; and Soria et al. Hepatology. 2013, vol. 57, no. 5, 2061-71, “Ammonia detoxification via ureagenesis in rat hepatocytes involves mitochondrial aquaporin-8 channels”).

By means of another example and not limitation, potassium (K⁺) transporters, such as Na⁺/K⁺-ATPase or the Na⁺—K⁺-2Cl⁻ cotransporter NKCC1, may be capable of transporting ammonium, since NH4′ has comparable or same ionic radius as and can compete with K+(see inter alia Hertz et al. Neurochem Res. 2015, vol. 40, no. 2, 241-57, “Ammonia, like K(+), stimulates the Na(+), K(+), 2 Cl(−) cotransporter NKCC1 and the Na(+),K(+)-ATPase and interacts with endogenous ouabain in astrocytes”; and Wall and Koger. Am J Physiol. 1994, 267, F660-70, “NH+4 transport mediated by Na(+)—K(+)-ATPase in rat inner medullary collecting duct”).

By means of another example and not limitation, NH4′ has been reported to substitute for H⁺in Na⁺/H⁺ exchanger (NHE), such as NHE2 and/or NHE3 (see inter alia Orlowski and Grinstein. Pflugers Arch. 2004, vol. 447, 549-65, “Diversity of the mammalian sodium/proton exchanger SLC9 gene family”; and Kinsella and Aronson. Am J Physiol. 1981, vol. 241, C220-6, “Interaction of NH4+ and Li+ with the renal microvillus membrane Na+-H+ exchanger”).

Accordingly, in certain embodiments, the at least one ammonium transporter is selected from the group consisting of RHBG, RHAG, RHCG, aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof.

In certain embodiments, the present methods, uses, or products may employ measurements of at least one ammonium transporter selected from the group consisting of RHBG, RHAG, RHCG, and combinations thereof.

In certain embodiments, the present methods, uses, or products may employ measurements of one ammonium transporter selected from the group consisting of RHBG, RHAG, and RHCG.

In certain embodiments, the present methods, uses, or products may employ measurements of two ammonium transporter selected from the group consisting of RHBG, RHAG, and RHCG, for example measurements of RHBG and RHAG, or RHBG and RHCG, or RHAG and RHCG.

In certain embodiments, the present methods, uses, or products may employ measurements of all three ammonium transporter selected from the group consisting of RHBG, RHAG, and RHCG.

The reference herein to any marker, peptide, polypeptide, protein, or nucleic acid also encompasses fragments thereof. Hence, the reference herein to measuring (or measuring the quantity of) any one marker, peptide, polypeptide, protein, or nucleic acid may encompass measuring the marker, peptide, polypeptide, protein, or nucleic acid, such as, e.g., measuring any mature and/or processed soluble/secreted form(s) thereof (e.g., plasma circulating form(s)) and/or measuring one or more fragments thereof.

For example, any marker, peptide, polypeptide, protein, or nucleic acid, and/or one or more fragments thereof may be measured collectively, such that the measured quantity corresponds to the sum amounts of the collectively measured species. In another example, any marker, peptide, polypeptide, protein, or nucleic acid, and/or one or more fragments thereof may be measured each individually.

The term “fragment” with reference to a peptide, polypeptide, or protein generally denotes a N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300 or ≥400 consecutive amino acids of the corresponding full-length peptide, polypeptide, or protein.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

The term encompasses fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The reference herein to any nucleic acid, protein, polypeptide or peptide may also encompass variants thereof. The term “variant” of a nucleic acid, protein, polypeptide or peptide refers to nucleic acids, proteins, polypeptides or peptides the sequence (i.e., nucleotide sequence or amino acid sequence, respectively) of which is substantially identical (i.e., largely but not wholly identical) to the sequence of said recited nucleic acid, protein or polypeptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited nucleic acid, protein, polypeptide or peptide when the whole sequence of the recited nucleic acid, protein, polypeptide or peptide is queried in the sequence alignment (i.e., overall sequence identity). Also included among fragments and variants of a nucleic acid, protein, polypeptide or peptide are fusion products of said nucleic acid, protein, polypeptide or peptide with another, usually unrelated, nucleic acid, protein, polypeptide or peptide.

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

A variant of a nucleic acid, protein, polypeptide or peptide may be a homologue (e.g., orthologue or paralogue) of said nucleic acid, protein, polypeptide or peptide. As used herein, the term “homology” generally denotes structural similarity between two macromolecules, particularly between two nucleic acids, proteins or polypeptides, from same or different taxons, wherein said similarity is due to shared ancestry.

Where the present specification refers to or encompasses fragments and/or variants of nucleic acids, proteins, polypeptides or peptides, this preferably denotes variants and/or fragments which are “functional”, i.e., which at least partly retain the biological activity or intended functionality of the respective nucleic acids, proteins, polypeptides or peptides. By means of example and not limitation, a functional fragment and/or variant of an ammonium transporter, such as RHBG, RHAG, or RHCG, shall at least partly retain the biological activity of said ammonium transporter, such as RHBG, RHAG, or RHCG, respectively, such as, e.g., ability to transport ammonia and/or ammonium into and/or from the cell, etc. Preferably, a functional fragment and/or variant may retain at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared to the corresponding nucleic acid, protein, polypeptide or peptide. For example, ammonium transport via an ammonium transporter, such as RHBG, RHAG, or RHCG, can be evaluated using stopped-flow spectrofluorimetry and the 2′,7′-bis-(2-carboxyethyl)-5(6)-carboxyfluorescein (BCECF) pH-sensitive probe to follow alterations in intracellular pH (pHᵢ) related to ammonium transport, substantially as described by Zidi-Yahiaoui et al. Human Rhesus B and Rhesus C glycoproteins: properties of facilitated ammonium transport in recombinant kidney cells. Biochem J. 2005; 391 (Pt 1): 33-40. Ammonium transport via an ammonium transporter, such as RHBG, RHAG, or RHCG, can further be evaluated by assays to determine apparent methylammonium permeability, substantially as described in Zidi-Yahiaoui et al. (supra) or Handlogten et al. Basolateral ammonium transport by the mouse inner medullary collecting duct cell (mIMCD-3). Am J Physiol Renal Physiol. 2004; 287(4): F628-38.

The term “proliferative disease or disorder” generally refers to any disease or disorder characterized neoplastic cell growth and proliferation, whether benign, pre-malignant, or malignant. The term proliferative disease generally includes all transformed cells and tissues and all cancerous cells and tissues. Proliferative diseases or disorders include, but are not limited to abnormal cell growth, benign tumours, premalignant or precancerous lesions, malignant tumours, and cancer. Examples of proliferative diseases and/or disorders are benign, pre-malignant, and malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

The proliferative disease or disorder may be a tumour or may be characterized by the presence of a tumour. As used herein, the terms “tumour” or “tumour tissue” refer to an abnormal mass of tissue that results from excessive cell division. A tumour or tumour tissue comprises “tumour cells” which are neoplastic cells with abnormal growth properties and no useful bodily function. Tumours, tumour tissue and tumour cells may be benign, pre-malignant or malignant, or may represent a lesion without any cancerous potential. A tumour or tumour tissue may also comprise “tumour-associated non-tumour cells”, e.g., vascular cells which form blood vessels to supply the tumour or tumour tissue. Non-tumour cells may be induced to replicate and develop by tumour cells, for example, the induction of angiogenesis in a tumour or tumour tissue.

The proliferative disease or disorder may be malignancy. As used herein, the term “malignancy” refers to a non-benign tumour or a cancer.

As used herein, the term “cancer” refers to a malignant neoplasm characterized by deregulated or unregulated cell growth. In certain embodiments, the proliferative disease or disorder may be selected from the group consisting of carcinoma, lymphoma, blastoma, sarcoma, leukemia, and lymphoid malignancies. In certain further embodiments, the proliferative disease or disorder may be selected from the group consisting of squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer or carcinoma, gastric or stomach cancer including gastrointestinal cancer, oesophageal cancer or carinoma, pancreatic cancer, glioma, glioblastoma, skin cancer, uterus cancer, cervical cancer, ovarian cancer, liver cancer, bile duct cancer, urothelial cancer, bladder cancer, hepatoma, breast cancer, colon cancer or carcinoma, rectal cancer, colorectal cancer, endometrial cancer or uterine carcinoma, salivary gland carcinoma, kidney or renal cancer or carcinoma, adrenal gland cancer, paraganglioma, prostate cancer, vulval cancer, thyroid cancer, hepatic carcinoma, anal carcinoma, penile carcinoma, multiple myeloma, brain cancer, and head and neck cancer. The term “cancer” includes primary malignant cells or tumours (e.g., those whose cells have not migrated to sites in the subject's body other than the site of the original malignancy or tumour) and secondary malignant cells or tumours (e.g., those arising from metastasis, the migration of malignant cells or tumour cells to secondary sites that are different from the site of the original tumour).

The proliferative disease or disorder may also be a premalignant condition. Premalignant conditions are known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell 1976 (Basic Pathology, 2d Ed., W. B. Saunders Co., Philadelphia, pp. 68-79).

In particular embodiments, the proliferative disease or disorder may be a hyperplastic disorder, a metaplastic disorder, or a dysplastic disorder.

The term “hyperplasia” refers to a form of controlled cell proliferation, involving an increase in cell number in a tissue or organ, without significant alteration in structure or function. In certain embodiments, the hyperplastic disorder may be selected from the group consisting of angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The term “metaplasia” refers to a form of controlled cell growth in which one type of adult or fully differentiated cell substitutes for another type of adult cell. In certain embodiments, the metaplastic disorder may be selected from the group consisting of agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The term “dysplasia” generally refers to an abnormality of cell development. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. In certain embodiments, the dysplastic disorder may be selected from the group consisting of anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders include, but are not limited to, benign dysproliferative disorders (e.g., benign tumours, fibrocystic conditions, tissue hypertrophy, intestinal polyps, colon polyps, and oesophageal dysplasia), leukoplakia, keratoses, Bowen's disease, Farmer's Skin, solar cheilitis, and solar keratosis.

In particular embodiments, the proliferative disease or disorder is selected form the group consisting of breast cancer, squamous cell cancer, lung cancer, cancer of the peritoneum, hepatocellular cancer or carcinoma, gastric cancer, stomach cancer, oesophageal cancer or carcinoma, pancreatic cancer, glioma, glioblastoma, skin cancer, uterus cancer, cervical cancer, ovarian cancer, liver cancer, urothelial cancer, bladder cancer, hepatoma, colon cancer or carcinoma, rectal cancer, colorectal cancer, endometrial cancer, uterine carcinoma, salivary gland carcinoma, renal cancer or carcinoma, prostate cancer, vulval cancer, thyroid cancer, hepatic carcinoma, anal carcinoma, penile carcinoma, leukemia, multiple myeloma, brain cancer, and head and neck cancer.

In particular embodiments, the proliferative disease or disorder is selected form the group consisting of hepatocellular carcinoma, hepatoma, hepatic carcinoma, liver cancer, colorectal cancer, colon cancer or carcinoma, and rectal cancer.

In particular embodiments, the proliferative disease or disorder is selected form the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, endometrial cancer, uterine carcinoma, salivary gland carcinoma, vulval cancer, thyroid cancer, anal carcinoma, penile carcinoma, head and neck cancer, adrenal gland cancer, paraganglioma, oesophageal cancer or carcinoma, kidney or renal cancer or carcinoma, urothelial cancer, bile duct cancer, prostate cancer, brain cancer, leukemia, and multiple myeloma.

In particular embodiments, the proliferative disease or disorder is selected form the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, adrenal gland cancer, bile duct cancer, paraganglioma, endometrial cancer, uterine carcinoma, salivary gland carcinoma, vulval cancer, thyroid cancer, anal carcinoma, penile carcinoma, and head and neck cancer.

In certain preferred embodiments, the proliferative disease may be characterised by (over)expression of said at least one ammonium transporter, such as preferably at least one ammonium transporter selected from the group consisting of RHBG, RHAG, RHCG, and combinations thereof, such as particularly preferably RHBG.

The term “expression” of a polypeptide by a cell generally refers to the production of said polypeptide by the cell. As commonly known, the expression of polypeptides by cells involves several successive molecular mechanisms, more particularly but without limitation, the transcription of a gene encoding said polypeptide into RNA, the polyadenylation and where applicable splicing and/or other post-transcriptional modifications of the RNA into mRNA, the localisation of the mRNA into cell cytoplasm, where applicable other post-transcriptional modifications of the mRNA, the translation of the mRNA into a polypeptide chain, where applicable post-translational modifications of the polypeptide, and folding of the polypeptide chain into the mature conformation of the polypeptide. For compartmentalised polypeptides, such as secreted polypeptides and transmembrane polypeptides, the production process further involves trafficking of the polypeptides, i.e., the cellular mechanism by which polypeptides are transported to the appropriate sub-cellular compartment or organelle, membrane, e.g. the plasma membrane, or outside the cell.

The term “overexpression” as used herein typically refers to an expression which is higher (more particularly, statistically significantly higher) than observed in the absence of the proliferative disease, such as in normal, healthy cells, particularly normal, healthy cells of the same tissue as that affected by the proliferative disease. By means of example, (over)expression of said at least one ammonium transporter, such as preferably at least one ammonium transporter selected from the group consisting of RHBG, RHAG, RHCG, and combinations thereof, such as particularly preferably RHBG may encompass an increase in the level of said at least one ammonium transporter, such as preferably at least one ammonium transporter selected from the group consisting of RHBG, RHAG, RHCG, and combinations thereof, such as particularly preferably RHBG (e.g., as measured by a suitable technique and expressed as a quantitative variable) in proliferative disease by at least about 10%, or by at least about 20%, or by at least about 30%, or by at least about 40%, or by at least about 50%, or by at least about 75%, or by at least about 100%, e.g., by at least about 150%, 200%, 250%, 300%, 400% or by at least about 500%, compared to normal, healthy cells, particularly normal, healthy cells of the same tissue as that affected by the proliferative disease.

Cancer cells typically display elevated glutaminolysis compared to non-cancerous or normal, healthy cells. Glutaminolysis, which catabolizes glutamine to generate ATP and lactate, is a metabolic pathway that involves the initial deamination of glutamine by GLS, yielding glutamate and ammonia. Glutaminolysis has critical roles in supporting macromolecule biosynthesis, regulating signalling pathways, and maintaining redox homeostasis, all of which contribute to cancer cell proliferation and survival.

Without wishing to be bound by any theory, the present inventors hypothesise that (over)expression of said at least one ammonium transporter, such as preferably at least one ammonium transporter selected from the group consisting of RHBG, RHAG, RHCG, and combinations thereof, such as particularly preferably RHBG by cancer cells may contribute to or facilitate their survival and/or proliferation, and thereby contribute to or facilitate the persistence and/or growth of cancer comprising the cells.

It shall be appreciated that cancer tissue comprises not only cancerous cells, but also a variety of non-cancerous cell types, such as connective tissue cells (e.g., stromal cells, fibroblasts), immune cells (e.g., T cells, macrophages, etc.), endothelial cells, etc. Hence, in certain embodiments, cell properties discussed in the present specification, such as quantity or expression level of said at least one ammonium transporter, such as preferably at least one ammonium transporter selected from the group consisting of RHBG, RHAG, RHCG, and combinations thereof, such as particularly preferably RHBG, or (over)expression of said at least one ammonium transporter, such as preferably at least one ammonium transporter selected from the group consisting of RHBG, RHAG, RHCG, and combinations thereof, such as particularly preferably RHBG, may be measured or determined on bulk cancer tissue, including cancerous and non-cancerous cell types. In certain other embodiments, said cell properties may be measured or determined on a subset of cells of the cancer tissue consisting essentially of or consisting of cancerous cells only. In certain other embodiments, said cell properties may be measured or determined on a subset of cells of the cancer tissue consisting essentially of or consisting of non-cancerous cell types only. Without wishing to be bound by any theory, metabolic reprogramming may occur in such non-cancerous cells, e.g., in cells of stroma, fibroblasts, and/or immune cell, which may lead to (over)-expression of said at least one ammonium transporter, such as preferably at least one ammonium transporter selected from the group consisting of RHBG, RHAG, RHCG, and combinations thereof, such as particularly preferably RHBG in said non-cancerous cells, thereby also favouring the proliferation of cancer cells.

Based on the realisation of the present inventors that at least one ammonium transporter selected from the group consisting of RHBG, RHAG, RHCG, and combinations thereof, such as particularly RHBG, can be used as a biomarker for proliferative diseases, the present inventors screened gene expression data and found that, besides in breast cancer, particularly luminal breast cancer, RHBG was also significantly (over)expressed or repressed in skin cancer, cervical cancer, uterus cancer, urothelial cancer, adrenocortical carcinoma, bladder urothelial carcinoma, breast invasive carcinoma, cervical squamous cell carcinoma, cholangiocarcinoma, colon adenomacarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, liver hepatocellular carcinoma, lung adenocarcinoma, lung squamous cell carcinoma, pheochromocytoma, paraganglioma, thyroid carcinoma, uterine carcinosarcoma, and uterine corpus endometrial carcinoma; that RHCG was significantly (over)expressed or repressed in adrenocortical carcinoma, bladder urothelial carcinoma, breast invasive carcinoma, cervical squamous cell carcinoma, colon adenomacarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, liver hepatocellular carcinoma, lung adenocarcinoma, lung squamous cell carcinoma, pancreatic adenocarcinoma, pheochromocytoma, paraganglioma, prostate adenocarcinoma, rectum adenocarcinoma, stomach adenocarcinoma, thyroid carcinoma, and uterine corpus endometrial carcinoma; and that RHAG was significantly (over)expressed or repressed in thyroid carcinoma and rectum adenocarcinoma, making these cancers particularly good candidates for the methods or agents for use as taught herein.

Accordingly, in particular embodiments, the proliferative disease characterised by (over)expression or repression of said at least one ammonium transporter is selected from the group consisting of breast cancer, luminal breast cancer, breast invasive carcinoma, skin cancer, cervical cancer, uterus cancer, urothelial cancer, adrenocortical carcinoma, bladder urothelial carcinoma, cervical squamous cell carcinoma, cholangiocarcinoma, colon adenomacarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, liver hepatocellular carcinoma, lung adenocarcinoma, lung squamous cell carcinoma, pancreatic adenocarcinoma, pheochromocytoma, paraganglioma, prostate adenocarcinoma, rectum adenocarcinoma, stomach adenocarcinoma, thyroid carcinoma, uterine carcinosarcoma, and uterine corpus endometrial carcinoma.

In particular embodiments, the proliferative disease characterised by (over)expression or repression of RHBG is selected from the group consisting of breast cancer, luminal breast cancer, skin cancer, cervical cancer, uterus cancer, urothelial cancer, adrenocortical carcinoma, bladder urothelial carcinoma, breast invasive carcinoma, cervical squamous cell carcinoma, cholangiocarcinoma, colon adenomacarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, liver hepatocellular carcinoma, lung adenocarcinoma, lung squamous cell carcinoma, pheochromocytoma, paraganglioma, thyroid carcinoma, uterine carcinosarcoma, and uterine corpus endometrial carcinoma, preferably selected from the group consisting of breast cancer, luminal breast cancer, skin cancer, cervical cancer, uterus cancer, urothelial cancer, adrenocortical carcinoma, bladder urothelial carcinoma, breast invasive carcinoma, cervical squamous cell carcinoma, cholangiocarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, lung adenocarcinoma, lung squamous cell carcinoma, pheochromocytoma, paraganglioma, thyroid carcinoma, uterine carcinosarcoma, and uterine corpus endometrial carcinoma.

In particular embodiments, the proliferative disease characterised by overexpression of RHBG is selected from the group consisting of breast cancer, luminal breast cancer, breast invasive carcinoma, hepatocellular carcinoma, hepatoma, hepatic carcinoma, liver cancer, liver hepatocellular, colorectal cancer, colon cancer or carcinoma, rectal cancer, colon adenocarcinoma, skin cancer, cervical cancer, cervical squamous cell carcinoma, uterus cancer, uterine carcinosarcoma, uterine corpus endometrial carcinoma, urothelial cancer, adrenocortical carcinoma, bladder urothelial carcinoma, kidney chromophobe, esophageal carcinoma, glioblastoma multiforme, acute myeloid leukemia, lung adenocarcinoma, lung squamous cell carcinoma, pheochromocytoma, paraganglioma, and thyroid carcinoma, preferably selected from the group consisting of breast cancer, luminal breast cancer, breast invasive carcinoma, skin cancer, cervical cancer, cervical squamous cell carcinoma, uterus cancer, uterine carcinosarcoma, uterine corpus endometrial carcinoma, urothelial cancer, adrenocortical carcinoma, bladder urothelial carcinoma, kidney chromophobe, esophageal carcinoma, glioblastoma multiforme, acute myeloid leukemia, lung adenocarcinoma, lung squamous cell carcinoma, pheochromocytoma, paraganglioma, and thyroid carcinoma.

In particular embodiments, the proliferative disease or disorder is breast cancer.

In certain preferred embodiments, the proliferative disease or disorder is luminal breast cancer.

The term “breast cancer” includes, for example, those conditions classified by biopsy or histology as malignant pathology. The clinical delineation of breast cancer diagnoses is well known in the medical arts. One of skill in the art will appreciate that breast cancer refers to any malignancy of the breast tissue, including, for example, carcinomas and sarcomas. Non-limiting examples of breast cancer include ductal carcinoma in situ (DCIS), lobular carcinoma in situ (LCIS), or mucinous carcinoma. Breast cancer also refers to infiltrating ductal (IDC) or infiltrating lobular carcinoma (ILC). Moreover, breast cancer also refers to all molecular subtypes of breast cancer, such as luminal A, luminal B, triple negative or basal(-like) and HER2 type breast cancer, of which luminal and basal breast cancer are the two most important ones.

The term “luminal breast cancer” as used herein refers to both luminal A and B subtypes. Luminal breast cancers arise from cells in the inner layer of the ducts that deliver the milk from the lobules to the nipple. Luminal breast cancer is typically oestrogen-receptor-positive (ER+) and low-grade, with luminal A tumours growing very slowly and luminal B tumours growing more quickly. Luminal A tumours have the best prognosis.

The terms “basal breast cancer” or “triple negative cancer” as used herein refers to tumours that are negative for three common markers, namely oestrogen receptor (ER), progestin receptor (PR) and human epidermal growth factor receptor-2 (HER-2). Basal breast cancers arise from cells in the outer layer of the ducts that deliver the milk from the lobules to the nipple. Basal breast cancer is typically aggressive, unresponsive to treatment and, ultimately, indicative of a poor prognosis.

As shown in the example section, luminal breast tumours display an increased expression level and protein quantity of RHBG when compared to basal breast tumours and control tissue.

The term “subject” or “patient” as used herein typically and preferably denotes humans, but may also encompass reference to non-human animals, preferably warm-blooded animals, more preferably vertebrates, even more preferably mammals, such as, e.g., non-human primates, rodents, canines, felines, equines, ovines, porcines, and the like. Particularly intended are subjects known or suspected to have a proliferative disease. Suitable subjects may include ones presenting to a physician for a screening for a proliferative disease and/or with symptoms and signs indicative of a proliferative disease.

In particular embodiments, the subject is diagnosed with or assumed to have cancer, preferably breast cancer.

In particular embodiments, at least one ammonium transporter, such as preferably at least one ammonium transporter selected from the group consisting of RHBG, RHAG, RHCG, and combinations thereof, such as particularly preferably RHBG is used as a biomarker for the diagnosis, prediction, prognosis and/or monitoring of said proliferative disease in the subject.

The terms “predicting” or “prediction”, “diagnosing” or “diagnosis” and “prognosticating” or “prognosis” are commonplace and well-understood in medical and clinical practice. It shall be understood that the phrase “a method for the diagnosis, prediction and/or prognosis” a given disease or condition may also be interchanged with phrases such as “a method for diagnosing, predicting and/or prognosticating” of said disease or condition or “a method for making (or determining or establishing) the diagnosis, prediction and/or prognosis” of said disease or condition, or the like.

By means of further explanation and without limitation, “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of” the diseases or conditions as taught herein in a subject may particularly mean that the subject has such, hence, is diagnosed as having such. “Diagnosis of no” diseases or conditions as taught herein in a subject may particularly mean that the subject does not have such, hence, is diagnosed as not having such. A subject may be diagnosed as not having such despite displaying one or more conventional symptoms or signs reminiscent of such.

The terms “prognosticating” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

Hence, prediction or prognosis of a disease or condition can inter alia allow to predict or make a prognosis of the occurrence of the disease or condition, or to predict or make a prognosis of the progression, aggravation, alleviation or recurrence of the disease or condition or response to treatment or to other external or internal factors, situations or stressors, etc.

A good prognosis of the condition as taught herein may generally encompass anticipation of a satisfactory partial or complete recovery from the conditions back to before the condition was obtained, preferably within an acceptable time period. A good prognosis of such may more commonly encompass anticipation of not further worsening or aggravating the general health of the patient, preferably within a given time period.

A poor prognosis of the diseases or conditions as taught herein may generally encompass anticipation of a limited recovery and/or unsatisfactorily slow recovery, or to substantially no recovery or even further worsening of such and more particularly resulting in death of the diseased subject.

Further, monitoring a disease or condition can inter alia allow to predict the occurrence of the disease or condition, or to monitor the progression, aggravation, alleviation or recurrence of the disease or condition, or response to treatment or to other external or internal factors, situations or stressors, etc. Advantageously, monitoring may be applied in the course of a medical treatment of a subject, preferably medical treatment aimed at alleviating the so-monitored disease or condition. Such monitoring may be comprised, e.g., in decision making whether a patient may be discharged, needs a change in treatment or needs further hospitalisation. As intended herein, a reference to monitoring of a disease or condition also specifically includes monitoring of the probability, risk or chance of a subject to develop the disease or condition, i.e., monitoring change(s) in said probability, risk or chance over time.

The present use of said at least one ammonium transporter, such as preferably at least one ammonium transporter selected from the group consisting of RHBG, RHAG, RHCG, and combinations thereof, such as particularly preferably RHBG may preferably allow for sensitivity and/or specificity (preferably, sensitivity and specificity) of at least 50%, at least 60%, at least 70% or at least 80%, e.g., ≥85% or ≥90% or ≥95%, e.g., between about 80% and 100% or between about 85% and 95%.

A further aspect of the invention thus relates to a method for the diagnosis, prediction, prognosis and/or monitoring of a proliferative disease in a subject or for determining whether a subject is in need of therapeutic or prophylactic treatment of a proliferative disease, comprising detecting at least one ammonium transporter in a tissue sample from the subject.

In certain embodiments, the at least one ammonium transporter belongs to the Rhesus (Rh) protein family.

In certain embodiments, the at least one ammonium transporter is selected from the group consisting of Rh family, B glycoprotein (RHBG), Rh family, A glycoprotein (RHAG), Rh family, C glycoprotein (RHCG), and combinations thereof.

In a related aspect, the invention provides a method for detecting or measuring the quantity or expression level of at least one ammonium transporter, such as preferably at least one ammonium transporter selected from the group consisting of RHBG, RHAG, RHCG, and combinations thereof, such as particularly preferably RHBG in a subject, said method comprising:
(i) obtaining a tissue sample from the subject; and
(ii) detecting or measuring the quantity or expression level of said at least one ammonium transporter in the tissue sample;
wherein said patient is affected by a proliferative disease.

In a related aspect, the invention provides a method for diagnosing and treating a proliferative disease in a subject comprising:
(i) detecting at least one ammonium transporter, such as preferably at least one ammonium transporter selected from the group consisting of RHBG, RHAG, RHCG, and combinations thereof, such as particularly preferably RHBG in a tissue sample from the subject;
(ii) diagnosing the subject as in need of treatment of the proliferative disease when said at least one ammonium transporter is detected in the tissue sample; and
(iii) administering an effective amount of a treatment to the diagnosed subject.

In particular embodiments, any one of the methods as taught herein may comprise the step of comparing the quantity or expression level of said at least one ammonium transporter, such as preferably at least one ammonium transporter selected from the group consisting of RHBG, RHAG, RHCG, and combinations thereof, such as particularly preferably RHBG in the sample with the reference quantity or expression level of said at least one ammonium transporter.

In particular embodiments, any one of the methods as taught herein may comprise the following steps:
(i) determining the quantity or expression level of at least one ammonium transporter, such as preferably at least one ammonium transporter selected from the group consisting of RHBG, RHAG, RHCG, and combinations thereof, such as particularly preferably RHBG in a tissue sample from the subject;
(ii) comparing the quantity or expression level of said at least one ammonium transporter as determined in (a) with a reference value, said reference value representing a known diagnosis, prediction and/or prognosis of said proliferative disease;
(iii) finding a deviation or no deviation of the quantity or expression level of said at least one ammonium transporter as determined in (a) from said reference value;
(iv) attributing said finding of deviation or no deviation to a particular diagnosis, prediction, or prognosis of the proliferative disease in the subject.

In a related aspect, the prevention provides a method for diagnosing and treating a proliferative disease in a subject comprising:
(i) determining the quantity or expression level of at least one ammonium transporter, such as preferably at least one ammonium transporter selected from the group consisting of RHBG, RHAG, RHCG, and combinations thereof, such as particularly preferably RHBG in a tissue sample from the subject;
(ii) comparing the quantity or expression level of said at least one ammonium transporter as determined in (i) with a reference value, said reference value representing a known diagnosis of said proliferative disease;
(iii) diagnosing the subject as in need of treatment of the proliferative disease when said quantity or expression level of said at least one ammonium transporter as determined in (i) deviates from said reference value, and
(iv) administering an effective amount of a treatment to the diagnosed subject.

In particular embodiments, any one of the methods as taught herein may comprise the following steps:
(i) determining the quantity or expression level of at least one ammonium transporter, such as preferably at least one ammonium transporter selected from the group consisting of RHBG, RHAG, RHCG, and combinations thereof, such as particularly preferably RHBG in a tissue sample from the subject;
(ii) comparing the quantity or expression level of said at least one ammonium transporter as determined in (a) with a reference value, said reference value representing a known diagnosis, prediction and/or prognosis of said proliferative disease;
(iii) finding a deviation or no deviation of the quantity or expression level of said at least one ammonium transporter as determined in (a) from said reference value;
(iv) attributing said finding of deviation or no deviation to a particular diagnosis, prediction, or prognosis of the proliferative disease in the subject,
wherein in step (iii) an elevated quantity or expression level of said at least one ammonium transporter in the tissue sample from the subject as compared to the reference value allows for the diagnosis, prediction, or prognosis of the proliferative disease in the subject.

In a related aspect, the invention provides a method for diagnosing and treating a proliferative disease in a subject comprising:
(i) determining the quantity or expression level of at least one ammonium transporter, such as preferably at least one ammonium transporter selected from the group consisting of RHBG, RHAG, RHCG, and combinations thereof, such as particularly preferably RHBG in a tissue sample from the subject;
(ii) comparing the quantity or expression level of said at least one ammonium transporter as determined in (i) with a reference value, said reference value representing a known diagnosis of said proliferative disease;
(iii) diagnosing the subject as in need of treatment of the proliferative disease when said quantity or expression level of said at least one ammonium transporter as determined in (i) deviates from said reference value, and
(iv) administering an effective amount of a treatment to the diagnosed subject,
wherein in step (iii) an elevated quantity or expression level of said at least one ammonium transporter in a tissue sample obtained from the subject as compared to the reference value allows for the diagnosis of the proliferative disease in the subject.

As used herein, a phrase such as “a subject in need of treatment” includes subjects that would benefit from treatment of a given condition, particularly proliferative diseases. Such subjects may include, without limitation, those that have been diagnosed with said condition, those prone to develop said condition and/or those in whom said condition is to be prevented.

In particular embodiments, the subject of interest is a human patient suspected of or actually diagnosed with a proliferative disease, preferably wherein said proliferative disease is breast cancer. In certain embodiments, the subject may be suspected of or actually diagnosed (e.g., by other means) with luminal breast cancer.

The terms “treat” or “treatment” encompass both the therapeutic treatment of an already developed disease or condition, such as the therapy of an already developed proliferative disease, as well as prophylactic or preventive measures, wherein the aim is to prevent or lessen the chances of incidence of an undesired affliction, such as to prevent occurrence, development and progression of proliferative diseases. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment. Non-limiting examples of therapeutic or prophylactic treatment of a proliferative disease are radiotherapy, chemotherapy, hormone therapy, biological therapy, bisphosphonate therapy, immune therapy, stem cell therapy, and surgery.

In particular embodiments, the treatment is selected from the group consisting of radiotherapy, chemotherapy, hormone therapy, biological therapy, bisphosphonate therapy, immune therapy, stem cell therapy, and surgery.

In particular embodiments, the treatment comprises administration of one or more of the therapeutic agents capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG, in accordance with certain aspects and embodiments of the present invention as disclosed elsewhere in this specification.

In particular embodiments, the therapeutic or prophylactic treatment is a therapeutic or prophylactic treatment for breast cancer. Non-limiting examples of medicinal products/active pharmaceutical ingredients for prevention of breast cancer are raloxifene hydrochloride or tamoxifen citrate. Non-limiting examples of medicinal products/active pharmaceutical ingredients for treatment of breast cancer are leuprolide acetate, methotrexate, plactitaxel (e.g., albumin-stabilized nanoparticle formulation), ado-trastuzumab emtansine, everolimus, anastrozole, pamidronate disodium, emestane, capecitabine, cyclophosphamide, docetaxel, doxorubicin hydrochloride, epirubicin hydrochloride, eribulin mesylate, everolimus, exemestane, fluorouracil (e.g., injection), toremifene, fluvestrant, letrozole, gemcitabine hydrochloride, goserelin acetate, trastuzumab, palbociclib, ixabepilone, ado-trastuzumab emtansine, lapatinib ditosylate, megestrol acetate, paclitaxel, palbociclib, pamidronate disodium, pertuzumab, thiotepa, toremifene and vinblastine sulfate.

In particular embodiments, the therapeutic or prophylactic treatment is a therapeutic or prophylactic treatment for luminal breast cancer, more preferably the therapeutic or prophylactic treatment is hormone therapy. Hormone therapy can treat luminal breast cancer, which is a hormone-receptor-positive (ER-positive and PR-positive) breast cancer, by lowering the amount of the hormone estrogen in the body or by blocking the action of estrogen on breast cancer cells by use of, for example, medicinal products which inhibit estrogen production by the ovaria, aromatase inhibitors which block estrogen production, selective estrogen receptor modulaters (SERMs) and estrogen receptor downregulators, Non-limiting examples of SERMs include tamoxifen, fulvestrant and toremifene. Non-limiting examples of aromatase inhibitors include anastrozole, letrozole and exemestane. Non-limiting examples of ovarian suppression medicinal products/active pharmaceutical ingredients include goserelin and leuprolide acetate.

In particular embodiments, the treatment is a combination of hormone therapy and one or more therapeutic agents capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG, in accordance with certain aspects and embodiments of the present invention as disclosed elsewhere in this specification.

The term “effective amount” as used herein may refer to a prophylactically effective amount, which is an amount of an active compound or pharmaceutical agent, more particularly a prophylactic agent, that inhibits or delays in a subject the onset of a disorder as being sought by a researcher, veterinarian, medical doctor or other clinician, or may refer to a therapeutically effective amount, which is an amount of active compound or pharmaceutical agent, more particularly a therapeutic agent, that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the agents as taught herein.

The term “administration” or “administering” as used herein refers to the giving of a certain treatment of a proliferative disease to a subject in need of such a treatment. Such a treatment can be a therapeutic or prophylactic agent according to the invention The route of administration may be essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

The present methods for the diagnosis, prediction, prognosis and/or monitoring of the proliferative disease may be adequately qualified as in vitro methods in that they apply one or more in vitro processing and/or analysis steps to a sample removed from the subject. The term “in vitro” generally denotes outside, or external to, a body, e.g., an animal or human body. Detecting said at least one ammonium transporter, such as preferably at least one ammonium transporter selected from the group consisting of RHBG, RHAG, RHCG, and combinations thereof, such as particularly preferably RHBG in a tissue sample from a subject may ordinarily imply that the examination phase of the present methods comprises measuring the quantity of said at least one ammonium transporter in the sample from the subject. One understands that the present methods may generally comprise an examination phase in which data is collected from and/or about the subject.

A molecule or analyte such as a marker, peptide, polypeptide, protein, or nucleic acid, is “detected” in a sample when the presence or absence and/or quantity of said molecule or analyte is detected or determined in the sample, preferably substantially to the exclusion of other molecules and analytes.

Depending on factors that can be evaluated and decided on by a skilled person, such as inter alia the type of a biomarker (e.g., peptide, polypeptide, protein, or nucleic acid), the type of a sample (e.g., whole blood, plasma, serum, cancer tissue biopsy, cancer tissue surgical specimen, histological section), the expected abundance of the biomarker in the sample, the type, robustness, sensitivity and/or specificity of the detection method used to detect the biomarker, etc., the biomarker may be measured directly in the sample, or the sample may be subjected to one or more processing steps aimed at achieving an adequate measurement of the biomarker.

By means of example, the sample may be subjected to one or more isolation or separation steps, aimed at whereby the biomarker is isolated from the sample or whereby a fraction of the sample is prepared which is enriched for the biomarker. For example, if the biomarker is a peptide, polypeptide, or protein, any known protein purification technique may be applied to the sample to isolate peptides, polypeptides, and proteins therefrom. If the biomarker is a nucleic acid, any known nucleic acid purification technique may be applied to the sample to isolate nucleic acids therefrom. Non-limiting examples of methods to purify peptides, polypeptides, proteins, or nucleic acids may include chromatography, preparative electrophoresis, centrifugation, precipitation, affinity purification, etc.

As used herein, the term “purified” with reference to markers, peptides, polypeptides, proteins, or nucleic acids does not require absolute purity. Instead, it denotes that such markers, peptides, polypeptides, proteins, or nucleic acids are in a discrete environment in which their abundance (conveniently expressed in terms of mass or weight or concentration) relative to other analytes is greater than in the biological sample. A discrete environment denotes a single medium, such as for example a single solution, gel, precipitate, lyophilisate, etc. Purified nucleic acids, proteins, polypeptides or peptides may be obtained by known methods including, for example, laboratory or recombinant synthesis, chromatography, preparative electrophoresis, centrifugation, precipitation, affinity purification, etc.

Purified markers, peptides, polypeptides, proteins, or nucleic acids may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Purity of peptides, polypeptides, or proteins may be determined by SDS-PAGE under reducing or non-reducing conditions using Coomassie blue or, preferably, silver stain. Quantity of nucleic acids may be determined by measuring absorbance A260. Purity of nucleic acids may be determined by measuring absorbance A260/A280, or by agarose- or polyacrylamide-gel electrophoresis and ethidium bromide or similar staining.

The terms “sample” or “biological sample” as used herein include any biological specimen obtained and isolated from a subject. Samples may include, without limitation, organ tissue (i.e., tumour tissue, more particular breast tumour tissue), whole blood, plasma, serum, whole blood cells, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions. Preferably, a sample may be readily obtainable by minimally invasive methods, such as blood collection or tissue biopsy, allowing the removal/isolation/provision of the sample from the subject. The term “tissue” as used herein encompasses all types of cells of the human body including cells of organs but also including blood and other body fluids recited above.

In particular embodiments, the sample is a tissue sample obtained from breast tissue, more preferably breast tumour tissue.

Any existing, available or conventional separation, detection and quantification methods may be used herein to measure the presence or absence (e.g., readout being present vs. absent; or detectable amount vs. undetectable amount) and/or quantity (e.g., readout being an absolute or relative quantity, such as, for example, absolute or relative concentration) of markers, peptides, polypeptides, proteins, or nucleic acids in samples.

For example, such methods may include biochemical assay methods, immunoassay methods, mass spectrometry analysis methods, or chromatography methods, or combinations thereof.

The term “immunoassay” generally refers to methods known as such for detecting one or more molecules or analytes of interest in a sample, wherein specificity of an immunoassay for the molecule(s) or analyte(s) of interest is conferred by specific binding between a specific-binding agent, commonly but without limitation an antibody, and the molecule(s) or analyte(s) of interest. Immunoassay technologies include without limitation immunohistochemistry, direct ELISA (enzyme-linked immunosorbent assay), indirect ELISA, sandwich ELISA, competitive ELISA, multiplex ELISA, radioimmunoassay (RIA), ELISPOT technologies, and other similar techniques known in the art. Principles of these immunoassay methods are known in the art, for example John R. Crowther, “The ELISA Guidebook”, 1st ed., Humana Press 2000, ISBN 0896037282.

By means of further explanation and not limitation, direct ELISA employs a labelled primary binding agent, e.g., antibody, to bind to and thereby quantify target antigen in a sample immobilised on a solid support such as a microwell plate. Indirect ELISA uses a non-labelled primary binding agent, e.g., antibody, which binds to the target antigen and a secondary labelled binding agent, e.g., antibody, that recognises and allows the quantification of the antigen-bound primary binding agent. In sandwich ELISA the target antigen is captured from a sample using an immobilised ‘capture’ binding agent, e.g., antibody, which binds to one antigenic site within the antigen, and subsequent to removal of non-bound analytes the so-captured antigen is detected using a ‘detection’ binding agent, e.g., antibody, which binds to another antigenic site within said antigen, where the detection binding agent may be directly labelled or indirectly detectable as above. Competitive ELISA uses a labelled ‘competitor’ that may either be the primary binding agent, e.g., antibody, or the target antigen. In an example, non-labelled immobilised primary binding agent, e.g., antibody, is incubated with a sample, this reaction is allowed to reach equilibrium, and then labelled target antigen is added. The latter will bind to the primary binding agent wherever its binding sites are not yet occupied by non-labelled target antigen from the sample. Thus, the detected amount of bound labelled antigen inversely correlates with the amount of non-labelled antigen in the sample. Multiplex ELISA allows simultaneous detection of two or more analytes within a single compartment (e.g., microplate well) usually at a plurality of array addresses (see, for example, Nielsen & Geierstanger 2004. J Immunol Methods 290: 107-20 and Ling et al. 2007. Expert Rev Mol Diagn 7: 87-98 for further guidance). As appreciated, labelling in ELISA technologies is usually by enzyme (such as, e.g., horse-radish peroxidase) conjugation and the end-point is typically colourimetric, chemiluminescent or fluorescent, magnetic, piezo electric, pyroelectric and other.

In particular embodiments, the at least one ammonium transporter protein, such as preferably at least one ammonium transporter proteins selected from the group consisting of RHBG, RHAG, RHCG, and combinations thereof, such as particularly preferably RHBG protein may be detected in a tissue sample obtained from a tumour, preferably a breast tumour, by an immunoassay technique (e.g., immunohistochemistry) capable of detecting said at least one ammonium transporter in cells comprised in the tissue sample obtained from the tumour. For applying the immunoassay technique (e.g., immunohistochemistry) thereon, the tissue sample(s) may be suitably processed as generally known in the art; for example, snap-frozen in liquid nitrogen, optionally kept at −80° C., and sectioned to allow for immuno-staining of frozen sections for said at least one ammonium transporter; or for example, suitably fixed (e.g., in 10% formalin), embedded in a matrix (e.g., in paraffin), sectioned, and treated to remove the matrix (e.g., deparaffinised), to allow for conventional histological evaluation and immuno-staining for the at least one ammonium transporter protein. The immuno-stained sections may be scanned by a suitable slide scanner at a suitable magnification (e.g., about 400×) to capture a 2D-image. The image, or a region thereof, or more commonly a representative number of regions of the image (e.g., at least 3, or at least 4, or at least 5 or at least 6 regions, such as for example between 3 and 12, or between 5 and 10, or between 6 and 8 regions), such as equally sized regions, wherein such regions may be selected using conventional image processing software, are subjected to conventional image analysis. The image analysis provides a suitable quantity representing the immuno-staining for the at least one ammonium transporter protein. In a preferred example, such quantity may be the total surface area of the staining for the at least one ammonium transporter protein in the image or region(s) thereof. Given that the number of cells present in the image or region(s) thereof may vary between samples and sections, the quantity representing the immuno-staining for the at least one ammonium transporter protein, such as the total surface area of the staining for the at least one ammonium transporter protein, can be suitably normalised. For example, the total surface area of the immuno-staining for the at least one ammonium transporter protein in the image or region(s) thereof may be divided by the total surface area of the cell nuclei in said image or said region(s) thereof, yielding a normalised surface area of the immuno-staining for the at least one ammonium transporter protein. For this purpose, cell nuclei can be stained as common in the art, e.g., using haematoxylin and eosin in conventional immunohistochemistry, and image analysed as explained above.

Radioimmunoassay (RIA) is a competition-based technique and involves mixing known quantities of radioactively-labelled (e.g., ¹²⁵I- or ¹³¹I-labelled) target antigen with binding agent, e.g., antibody, to said antigen, then adding non-labelled or ‘cold’ antigen from a sample and measuring the amount of labelled antigen displaced (see, e.g., “An Introduction to Radioimmunoassay and Related Techniques”, by Chard T, ed., Elsevier Science 1995, ISBN 0444821198 for guidance).

Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), are useful herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ⁿ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)ⁿ; atmospheric pressure photoionization mass spectrometry (APPI-MS); APPI-MS/MS; and APPI-(MS)ⁿ. Peptide ion fragmentation in tandem MS (MS/MS) arrangements may be achieved using manners established in the art, such as, e.g., collision induced dissociation (CID). Detection and quantification of biomarkers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods described herein below.

Chromatography may also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably HPLC. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilised metal affinity chromatography, and the like.

Chromatography, including single-, two- or more-dimensional chromatography, may be used as a peptide fractionation method in conjunction with a further peptide analysis method, such as for example, with a downstream mass spectrometry analysis as described elsewhere in this specification.

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

The level of biomarkers at the nucleic acid level, more particularly RNA level, e.g., at the level of hnRNA, pre-mRNA, mRNA, or cDNA, may be detected using standard quantitative RNA or cDNA measurement tools known in the art. Non-limiting examples include hybridisation-based analysis, microarray expression analysis, digital gene expression (DGE), RNA-in-situ hybridisation (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, or the like.

Numerous different PCR or qPCR protocols are known in the art. Generally, in PCR, a target polynucleotide sequence is amplified by reaction with a pair of oligonucleotide primers. The primers hybridise to complementary regions of a target nucleic acid and a DNA polymerase extends the primers to amplify the target sequence, generating an amplification product. The amplification cycle is repeated to increase the concentration of the amplification product.

The reaction can be performed in any thermocycler commonly used for PCR. However, preferred are cyclers with real-time fluorescence measurement capabilities, for example, Smartcycler® (Cepheid, Sunnyvale, Calif.), ABI PRISM 7700® (Applied Biosystems, Foster City, Calif.), Rotor-Gene™ (Corbett Research, Sydney, Australia), Lightcycler® (Roche Diagnostics Corp, Indianapolis, Ind.), iCycler® (Biorad Laboratories, Hercules, Calif.), MX4000® (Stratagene, La Jolla, Calif.), and CFX96 Real-Time PCR system (Biorad).

As used herein, “quantitative PCR” (or “real-time qPCR”) refers to the direct monitoring of the progress of a PCR amplification as it is occurring without the need for repeated sampling of the reaction products. In QPCR, the reaction products may be monitored via a signalling mechanism (e.g., fluorescence) as they are generated and are tracked after the signal rises above a background level but before the reaction reaches a plateau. The number of cycles required to achieve a detectable or “threshold” level of fluorescence (“cycle threshold”, “CT”) varies directly with the concentration of amplifiable targets at the beginning of the PCR process, enabling a measure of signal intensity to provide a measure of the amount of target nucleic acid in a sample in real time.

By means of example and not limitation, real-time amplification, especially real-time PCR, as intended herein encompasses fully conventional systems, such as, e.g., the TaqMan™ system developed by Applied Biosystems, which relies on the release and detection of a fluorogenic probe during each round of DNA amplification (Holland et al. 1991. Detection of specific polymerase chain reaction product by utilizing the 5′-3′ exonuclease activity of Thermus aquaticus DNA polymerase. PNAS 88: 7276-80). The method uses the 5′ exonuclease activity of Taq polymerase during primer extension to cleave a dual-labelled, fluorogenic probe hybridised to the target DNA between the PCR primers. Prior to cleavage, a reporter fluorophore, such as 6-carboxyfluorescein (6-FAM) at the 5′ end of the probe is quenched by 6-carboxy-tetramethylrhodaniine (TAMRA) through fluorescent resonance energy transfer (FRET). Following digestion, FAM is released. The resulting fluorescence measured in real-time at around 518 nm during the log phase of product accumulation is proportional to the number of copies of the target sequence.

Further real-time amplification, especially real-time PCR, detection systems can also utilise FRET, such as, e.g., systems based on molecular beacons. Molecular beacons are single-stranded polynucleotide probes that possess a stem-and-loop hairpin structure. The loop portion is a probe sequence complementary to a sequence within an amplicon to be evaluated, and the stem is formed by short complementary sequences located at the opposite ends of the molecular beacon. The molecular beacon is labelled with a fluorophore (e.g., 6-FAM) at one end and a quencher (e.g., TAMRA) at the other end. When free in solution, the stem keeps the fluorophore and the quencher in close proximity, causing the fluorescence of the fluorophore to be quenched by FRET. However, when bound to its complementary target, the probe-target hybrid forces the stem to unwind, separating the fluorophore from the quencher, and restoring the fluorescence. Accordingly, when the quantity of an amplicon increases during amplification, this can be monitored as an increase in the fluorescence of the corresponding beacon (see, e.g., Manganelli et al. 2001. Real-time PCR using molecular beacons. Methods Mol Med 54: 295-310; Marras S A E. 2006. Selection of fluorophore and quencher pairs for fluorescent nucleic acid hybridization probes. Methods Mol Biol 335: 3-16; Marras S A E et al. 2006. Real-time assays with molecular beacons and other fluorescent nucleic acid hybridization probes. Clin Chim Acta 363: 48-60 for further discussion of molecular beacons detection).

An alternative real-time PCR amplification and detection system is the Light Upon Extension (LUX™) system commercialised by Invitrogen (Carlsbad, Calif.) and described in detail in Nazarenko et al. 2002 (Nucleic Acids Research 30: e37) and Nazarenko et al. 2002 (Nucleic Acids Research 30: 2089-2095). This system employs primer pairs in which usually one of the primers of said primer pair is labelled by a fluorophore (such as, e.g., FAM or JOE or Alexa Fluor 546). The particular structure of the “free” primer quenches the signal of the fluorophore bound thereto, whereas the fluorophore's signal intensity increases when the primer assumes an extended conformation once incorporated into the amplification product.

The sequence of primers may be tailored to perform with the LUX™ technology, following instructions of the above publications of Nazarenko et al. 2002 or using software tools provided by Invitrogen on www.invitrogen.com/lux. The LUX™ technology is particularly well suited for multiplexing (i.e., performing in a single reaction) of two or more amplifications using different primer sets, since each of the primer sets may be marked using a different fluorophore.

For description of additional ways to detect and evaluate amplification products in real-time (e.g., using adjacent probes; 5′-nuclease probes such as Taqman™; Light-up probes; Duplex scorpion primers; Amplifluor primers; and further alternative fluorescent hybridisation probe formats see, e.g., Marras S A E et al. 2006. Real-time assays with molecular beacons and other fluorescent nucleic acid hybridization probes. Clin Chim Acta 363: 48-60, esp. section 6 and references therein).

In particular embodiments, the method for diagnosing, prediction, prognosis and/or monitoring of a proliferative disease in a subject or for determining whether a subject is in need of therapeutic or prophylactic treatment according to the invention comprises the detection of said at least one ammonium transporter, such as preferably at least one ammonium transporter selected from the group consisting of RHBG, RHAG, RHCG, and combinations thereof, such as particularly preferably RHBG in a tissue sample by an immunohistochemistry assay or by RT-qPCR.

Various techniques for measuring biomarkers may employ binding agents for said respective biomarkers. Hence, further disclosed are binding agents capable of specifically binding to markers, peptides, polypeptides, proteins, or nucleic acids as taught herein. Binding agents as intended throughout this specification may include inter alia an antibody or antibody fragment, aptamer, photoaptamer, protein, peptide, peptidomimetic, nucleic acid such as an oligonucleotide, or a small molecule.

As used herein, the term “agent” broadly refers to any chemical (e.g., inorganic or organic), biochemical or biological substance, compound, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, peptides, peptidomimetics, antibodies and fragments and derivatives thereof, aptamers, photoaptamers, chemical substances, preferably organic molecules, more preferably small organic molecules, lipids, carbohydrates, polysaccharides, etc., and any combinations thereof. The term “bind”, “interact”, “specifically bind” or “specifically interact” as used throughout this specification means that an agent binds to or influences one or more desired molecules or analytes substantially to the exclusion of other molecules which are random or unrelated, and optionally substantially to the exclusion of other molecules that are structurally related. The term “bind”, “interact”, “specifically bind” or “specifically interact” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

The binding or interaction between the agent and its intended target(s) may be covalent (i.e., mediated by one or more chemical bonds that involve the sharing of electron pairs between atoms) or, more typically, non-covalent (i.e., mediated by non-covalent forces, such as for example, hydrogen bridges, dipolar interactions, van der Waals interactions, and the like). Preferably, the agent may bind to or interact with its intended target(s) with affinity constant (K_{A}) of such binding K_{A}≥1×10⁶ M⁻¹, more preferably K_{A}≥1×10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹, wherein K_{A}=[A_T]/[A][T], A denotes the agent, T denotes the intended target. Determination of K_{A} can be carried out by methods known in the art, such as for example, using equilibrium dialysis and Scatchard plot analysis.

The term “protein” as used herein generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used herein generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

Reference to protein- and polypeptide agents also includes without limitation naturally-occurring binding and/or regulatory partners of said at least one ammonium transporter, such as preferably at least one ammonium transporter selected from the group consisting of RHBG, RHAG, RHCG, and combinations thereof, such as particularly preferably RHBG, and variants or fragments thereof, including intracellular, transmembrane and extracellular binding and/or regulatory partners of said d at least one ammonium transporter. By means of an example, the existence of an RHBG regulatory partner has been demonstrated for the yeast homologue of RHBG (see Boeckstaens et al. Identification of a Novel Regulatory Mechanism of Nutrient Transport Controlled by TORC1-Npr1-Amu1/Par32. PLoS Genet. 2015 July; 11(7): e1005382), which is hypothesized to mediate the inactivation of the ammonium transporter by binding to the cytoplasmic side of the transporter. Naturally-occurring binding and/or regulatory partners of said at least one ammonium transporter from other species, such as of human ammonium transporter, can be readily identified by standard protein-protein interaction (PPI) detection methods, such as co-immunoprecipitation, phage display, chemical cross-linking, or yeast two hybrid screens.

The term “peptide” as used herein preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “peptidomimetic” as used herein refers to a non-peptide agent that is a topological analogue of a corresponding peptide. Methods of rationally designing peptidomimetics of peptides are known in the art. For example, by means of a guidance, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “nucleic acid” as used herein typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Exemplary modified nucleobases include without limitation 5-substituted pyrimidines, 6-azapyrimidines and N-2, N-6 and O-6 substituted purines, including 2-aminopropyladenine, 5-propynyluracil and 5-propynylcytosine. In particular, 5-methylcytosine substitutions have been shown to increase nucleic acid duplex stability and may be preferred base substitutions in for example antisense agents, even more particularly when combined with 2′-O-methoxyethyl sugar modifications. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups (such as without limitation 2′-O-alkylated, e.g., 2′-O-methylated or 2′-O-ethylated sugars such as ribose; 2′-O-alkyloxyalkylated, e.g., 2′-O-methoxyethylated sugars such as ribose; or 2′-O,4′-C-alkylene-linked, e.g., 2′-O,4′-C-methylene-linked or 2′-O,4′-C-ethylene-linked sugars such as ribose; 2′-fluoro-arabinose, etc.). Nucleoside units may be linked to one another by any one of numerous known inter-nucleoside linkages, including inter alia phosphodiester linkages common in naturally-occurring nucleic acids, and further modified phosphate- or phosphonate-based linkages such as phosphorothioate, alkyl phosphorothioate such as methyl phosphorothioate, phosphorodithioate, alkylphosphonate such as methylphosphonate, alkylphosphonothioate, phosphotriester such as alkylphosphotriester, phosphoramidate, phosphoropiperazidate, phosphoromorpholidate, bridged phosphoramidate, bridged methylene phosphonate, bridged phosphorothioate; and further siloxane, carbonate, sulfamate, carboalkoxy, acetamidate, carbamate such as 3′-N-carbamate, morpholino, borano, thioether, 3′-thioacetal, and sulfone internucleoside linkages.

Preferably, inter-nucleoside linkages may be phosphate-based linkages including modified phosphate-based linkages, such as more preferably phosphodiester, phosphorothioate or phosphorodithioate linkages or combinations thereof. The term “nucleic acid” also encompasses any other nucleobase containing polymers such as nucleic acid mimetics, including, without limitation, peptide nucleic acids (PNA), peptide nucleic acids with phosphate groups (PHONA), locked nucleic acids (LNA), morpholino phosphorodiamidate-backbone nucleic acids (PMO), cyclohexene nucleic acids (CeNA), tricyclo-DNA (tcDNA), and nucleic acids having backbone sections with alkyl linkers or amino linkers (see, e.g., Kurreck 2003 (Eur J Biochem 270: 1628-1644)). “Alkyl” as used herein particularly encompasses lower hydrocarbon moieties, e.g., C1-C4 linear or branched, saturated or unsaturated hydrocarbon, such as methyl, ethyl, ethenyl, propyl, 1-propenyl, 2-propenyl, and isopropyl. Nucleic acids as intended herein may include naturally occurring nucleosides, modified nucleosides or mixtures thereof. A modified nucleoside may include a modified heterocyclic base, a modified sugar moiety, a modified inter-nucleoside linkage or a combination thereof. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear. Nucleic acids and particularly antisense oligonucleotides or RNAi agents may be herein denoted as comprising uracil (U) bases. It shall be appreciated that U may be optionally substituted by thymine (T) in (at least some) such nucleic acids and agents. For example, as 2′-O-methyl phosphorothioate antisense oligonucleotides are more ‘RNA-like’, U may be used and denoted in such molecules. With other antisense chemistries, such as peptide nucleic acids or morpholino backbones, T bases may be preferably denoted and used.

The term “aptamer” as used herein refers to single-stranded or double-stranded oligo-DNA, oligo-RNA or oligo-DNA/RNA or any analogue thereof that can specifically bind to a target molecule. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term also encompasses photoaptamers, i.e., aptamers that contain one or more photoreactive functional groups that can covalently bind to or crosslink with a target molecule.

The term “small organic molecule” or “small molecule” as used herein encompasses organic compounds with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

As used herein, the term “antibody” is used in its broadest sense and generally refers to any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The term antibody thereby includes a whole immunoglobulin molecule, a monoclonal antibody, a chimeric antibody, a humanized antibody, a human antibody, or an immunologically effective fragment of any of these. The term thus specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

The term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acid sequences or nucleotide sequences encoding the same, unless the context requires a more limited interpretation.

The term “epitope” includes any polypeptide determinant capable of specifically binding to an immunoglobulin or T-cell receptor. Epitope determinants may include chemically active surface groupings of molecules such as amino acids, sugar side chains, phosphoryl, or sulfonyl, and may have specific three dimensional structural characteristics, and/or specific charge characteristics. An epitope is a region of an antigen that is bound by an antibody. An antibody is said to specifically bind an antigen when it preferentially recognizes its target antigen in a complex mixture of proteins and/or macromolecules.

The terms “binding region”, “binding site” or “interaction site” shall herein have the meaning of a particular site, part, domain or stretch of amino acid residues that is responsible for binding to an antigen of interest. Such binding region essentially consists of specific amino acid residues of the antibodies described herein, which residues are in contact with the target molecule.

The term “specificity” refers to the number of different types of antigens or antigenic determinants to which a particular antigen-binding molecule or antigen-binding protein (such as an antibody) molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to the skilled person, affinity can be determined in a manner known per se, depending on the specific antigen of interest. Avidity is the measure of the strength of binding between an antigen-binding molecule (such as an antibody) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as antibodies) will bind with a dissociation constant (KD) of 10⁻⁵ to 10⁻¹² moles/liter (M) or less, and preferably 10⁻⁷ to 10⁻¹² moles/liter (M) or less and more preferably 10⁻⁸ to 10⁻¹² moles/liter, and/or with an association constant(KA) of at least 10⁷ M⁻¹, preferably at least 10⁸ M⁻¹, more preferably at least 10⁹ M⁻¹, such as at least 10¹² M⁻¹. Any KD value greater than 10⁻⁴ M is generally considered to indicate non-specific binding. Preferably, an antibody will bind to the desired antigen with an KD less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

A full-length antibody as it exists naturally is an immunoglobulin molecule comprising 2 heavy (H) chains and 2 light (L) chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (FR). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions is in accordance with the well-known Kabat numbering convention, which refers to a system of numbering amino acid residues which are more variable (i.e., hypervariable) than other amino acid residues in the heavy and light chain regions of an antibody (Kabat, et al., Ann. NY Acad. Sci. 190:382-93 (1971); Kabat, et al., Sequences of Proteins of Immunological Interest, Fifth Edition, U.S. Department of Health and Human Services, NIH Publication No. 91-3242 (1991)). The positioning of CDRs in the variable region of an antibody follows Kabat numbering or simply, “Kabat.”

Light chains are classified as kappa or lambda, and are characterized by a particular constant region as known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

In particular embodiments, an antibody may be any of IgA, IgD, IgE, IgG and IgM classes, and preferably IgG class antibody.

The term “polyclonal antibody” as used herein may be an antiserum or immunoglobulins purified there from (e.g., affinity-purified).

As used herein, the term “monoclonal antibody” refers to an antibody that is derived from a single copy or clone including, for example, any eukaryotic, prokaryotic, or phage clone, and not the method by which it is produced. Monoclonal antibodies can target a particular antigen or a particular epitope within an antigen with greater selectivity and reproducibility. Monoclonal antibodies preferably exist in a homogeneous or substantially homogeneous population. Monoclonal antibodies and antigen-binding fragments thereof of the present invention can be produced, for example, by recombinant technologies, phage display technologies, synthetic technologies, e.g., CDR-grafting, or combinations of such technologies, or other technologies known in the art.

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be made using phage antibody libraries using techniques as described by Clackson et al. 1991 (Nature 352: 624-628) and Marks et al. 1991 (J Mol Biol 222: 581-597).

These latter techniques are based on the phage display technology as described inter alia in U.S. Pat. Nos. 5,837,500, 5,571,698, 5,223,409, 7,118,879, 7,208,293 and 7,413,537. Briefly, therapeutic candidate molecules, e.g., human antibody fragments (e.g., Fabs), peptides, and small proteins, are displayed on the surface of a small bacterial virus called a bacteriophage (or phage). A collection of displayed molecules is known as a library. Phage display enables to search through these libraries to identify molecules that bind, preferably with high specificity and/or affinity, to targets of interest, e.g. therapeutic targets. Non-limiting examples of phage antibody libraries include HuCAL® (Human Combinatorial Antibody Library, Morphosys), Ylanthia® (Morphosys), the human Fab fragment libraries described in WO200070023, and the macaque antibody library as described in WO 1996040878. The Human Combinatorial Antibody Library (Morphosys) has been prepared as described in WO 199708320 using synthetic consensus sequences which cover the structural repertoire of antibodies encoded in the human genome.

In certain embodiments, the binding agent may be an antibody fragment.

The term “antibody fragment” or “antigen-binding moiety” comprises a portion or region of a full length antibody, generally the antigen binding or variable domain thereof. Examples of antibody fragments include Fab, Fab′, F(ab)2, Fv, sFv fragments, single domain (sd)Fv, such as V_{H} domains, V_{L} domains and V_{HH} domains, diabodies, linear antibodies, single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term “antigen-binding portion” or “antigen-binding region” refers to one or more fragments of an antibody that retain the ability to specifically bind to an antigen. It has been shown that the antigen-binding function of an antibody may be performed by fragments of a full-length antibody. These may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., Nature, 341: 544-546 (1989); PCT publication WO 90/05144), which comprises a single variable domain; and (vi) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain Fv (scFv) (Bird et al., Science, 242: 423-426 (1988); and Huston et al., Proc. Natl. Acad. Sci., 85: 5879-5883 (1988)). Such single chain antibodies are also intended to be encompassed within the term “antigen-binding portion” of an antibody. Other forms of single chain antibodies, such as diabodies are also encompassed.

Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (Holliger, et al., Proc. Natl. Acad. Sci., 90: 6444-6448 (1993); Poljak, et al., Structure 2: 1121-1123 (1994)). Such antibody binding portions are known in the art (Kontermann and Dubel eds., Antibody Engineering (2001) Springer-Verlag. New York. 790 pp. (ISBN 3-540-41354-5).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al., Human Antibodies and Hybridomas, 6: 93-101 (1995)) and use of a cysteine residue, a marker peptide and a C-terminal polyhistidine tag to make bivalent and biotinylated scFv molecules (Kipriyanov, et al., Mol. Immunol., 31: 1047-1058 (1994)). Antibody portions, such as Fab and F(ab′)2 fragments, may be prepared from whole antibodies using conventional techniques, such as papain or pepsin digestion, respectively, of whole antibodies. Moreover, antibodies, antibody portions and immunoadhesion molecules may be obtained using standard recombinant DNA techniques.

In particular embodiments, the binding agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a naturally occurring heavy-chain antibody, preferably a heavy-chain antibody derived from camelids; (2) by expression of a nucleotide sequence encoding a naturally occurring V_{HH} domain; (3) by “humanization” of a naturally occurring V_{HH} domain or by expression of a nucleic acid encoding a such humanized V_{HH} domain; (4) by “camelization” of a naturally occurring V_{H} domain from any animal species, and in particular from a mammalian species, such as from a human being, or by expression of a nucleic acid encoding such a camelized V_{H} domain; (5) by “camelisation” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

The amino acid sequence and structure of a Nanobody can be considered—without however being limited thereto—to be comprised of four framework regions or “FR's”, which are referred to in the art and herein as “Framework region 1” or “FRI”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region I” or “CDRI”; as “Complementarity Determining Region 2” or “CDR2”; and as “Complementarity Determining Region 3” or “CDR3”, respectively. The total number of amino acid residues in a Nanobody can be in the region of 110-120, and preferably 112-115. It should however be noted that parts, fragments, analogs or derivatives of a Nanobody are not particularly limited as to their length and/or size, as long as such parts, fragments, analogs or derivatives meet the further requirements outlined herein and are preferably suitable for the purposes described herein.

The amino acid residues of a Nanobody are numbered according to the general numbering for VH domains given by Kabat et al. (“Sequence of proteins of immunological interest”, US Public Health Services, NIH Bethesda, Md., Publication No. 91). According to this numbering, FRI of a Nanobody comprises the amino acid residues at positions 1-30, CDRI of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for VH domains and for VHH domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FRI and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.]. Alternative methods for numbering the amino acid residues of VH domains, which methods can also be applied in an analogous manner to VHH domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to VHH domains by Riechmann and Muyldermans will be followed, unless indicated otherwise.

For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and applicant; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by applicant and the further published patent applications by applicant; Hamers-Casterman et al., Nature 1993 Jun. 3; 363 (6428): 446-8; Davies and Riechmann, FEBS Lett. 1994 Feb. 21; 339(3): 285-90; Muyldermans et al., Protein Eng. 1994 September; 7(9): 1129-3; Davies and Riechmann, Biotechnology (NY) 1995 May; 13(5): 475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9(6): 531-7; Desmyter et al., Nat Struct Biol. 1996 September; 3(9): 803-11; Sheriff et al., Nat Struct Biol. 1996 September; 3(9): 733-6; Spinelli et al., Nat Struct Biol. 1996 September; 3(9): 752-7; Arbabi Ghahroudi et al., FEBS Lett. 1997 Sep. 15; 414(3): 521-6; Vu et al., Mol Immunol. 1997 November-December; 34(16-17): 1121-31; Atarhouch et al., Journal of Camel Practice and Research 1997; 4: 177-182; Nguyen et al., J. Mol. Biol. 1998 Jan. 23; 275(3): 413-8; Lauwereys et al., EMBO J. 1998 Jul. 1; 17(13): 3512-20; Frenken et al., Res Immunol. 1998 July-August; 149(6): 589-99; Transue et al., Proteins 1998 Sep. 1; 32(4): 515-22; Muyldermans and Lauwereys, J. Mol. Recognit. 1999 March-April; 12 (2): 131-40; van der Linden et al., Biochim. Biophys. Acta 1999 Apr. 12; 1431(1): 37-46; Decanniere et al., Structure Fold. Des. 1999 Apr. 15; 7(4): 361-70; Ngyuen et al., Mol. Immunol. 1999 June; 36(8): 515-24; Woolven et al., Immunogenetics 1999 October; 50 (1-2): 98-101; Riechmann and Muyldermans, J. Immunol. Methods 1999 Dec. 10; 231 (1-2): 25-38; Spinelli et al., Biochemistry 2000 Feb. 15; 39(6): 1217-22; Frenken et al., J. Biotechnol. 2000 Feb. 28; 78(1): 11-21; Nguyen et al., EMBO J. 2000 Mar. 1; 19(5): 921-30; van der Linden et al., J. Immunol. Methods 2000 Jun. 23; 240 (1-2): 185-95; Decanniere et al., J. Mol. Biol. 2000 Jun. 30; 300 (1): 83-91; van der Linden et al., J. Biotechnol. 2000 Jul. 14; 80(3): 261-70; Harmsen et al., Mol. Immunol. 2000 August; 37(10): 579-90; Perez et al., Biochemistry 2001 Jan. 9; 40(1): 74-83; Conrath et al., J. Biol. Chem. 2001 Mar. 9; 276 (10): 7346-50; Muyldermans et al., Trends Biochem Sci. 2001 April; 26(4):230-5; Muyldermans S., J. Biotechnol. 2001 June; 74 (4): 277-302; Desmyter et al., J. Biol. Chem. 2001 Jul. 13; 276 (28): 26285-90; Spinelli et al., J. Mol. Biol. 2001 Aug. 3; 311 (1): 123-9; Conrath et al., Antimicrob Agents Chemother. 2001 October; 45 (10): 2807-12; Decanniere et al., J. Mol. Biol. 2001 Oct. 26; 313(3): 473-8; Nguyen et al., Adv Immunol. 2001; 79: 261-96; Muruganandam et al., FASEB J. 2002 February; 16 (2): 240-2; Ewert et al., Biochemistry 2002 Mar. 19; 41 (11): 3628-36; Dumoulin et al., Protein Sci. 2002 March; 11 (3): 500-15; Cortez-Retamozo et al., Int. J. Cancer. 2002 Mar. 20; 98 (3): 456-62; Su et al., Mol. Biol. Evol. 2002 March; 19 (3): 205-15; van der Vaart J M., Methods Mol Biol. 2002; 178: 359-66; Vranken et al., Biochemistry 2002 Jul. 9; 41 (27): 8570-9; Nguyen et al., Immunogenetics 2002 April; 54 (1): 39-47; Renisio et al., Proteins 2002 Jun. 1; 47 (4): 546-55; Desmyter et al., J. Biol. Chem. 2002 Jun. 28; 277 (26): 23645-50; Ledeboer et al., J. Dairy Sci. 2002 June; 85 (6): 1376-82; De Genst et al., J. Biol. Chem. 2002 Aug. 16; 277 (33): 29897-907; Ferrat et al., Biochem. J. 2002 Sep. 1; 366 (Pt 2): 415-22; Thomassen et al., Enzyme and Microbial Technol. 2002; 30: 273-8; Harmsen et al., Appl. Microbiol. Biotechnol. 2002 December; 60 (4): 449-54; Jobling et al., Nat Biotechnol. 2003 January; 21 (1): 77-80; Conrath et al., Dev. Comp. Immunol. 2003 February; 27 (2): 87-103; Pleschberger et al., Bioconjug. Chem. 2003 March-April; 14 (2): 440-8; Lah et al., J. Biol. Chem. 2003 Apr. 18; 278 (16): 14101-11; Nguyen et al., Immunology. 2003 May; 109 (1): 93-101; Joosten et al., Microb. Cell Fact. 2003 Jan. 30; 2 (1): 1; Li et al., Proteins 2003 Jul. 1; 52 (1): 47-50; Loris et al., Biol Chem. 2003 Jul. 25; 278 (30): 28252-7; van Koningsbruggen et al., J. Immunol. Methods. 2003 August; 279 (1-2): 149-61; Dumoulin et al., Nature. 2003 Aug. 14; 424 (6950): 783-8; Bond et al., J. Mol. Biol. 2003 Sep. 19; 332 (3): 643-55; Yau et al., J. Immunol. Methods. 2003 Oct. 1; 281 (1-2): 161-75; Dekker et al., J. Virol. 2003 November; 77 (22): 12132-9; Meddeb-Mouelhi et al., Toxicon. 2003 December; 42 (7): 785-91; Verheesen et al., Biochim. Biophys. Acta 2003 Dec. 5; 1624 (1-3): 21-8; Zhang et al., J Mol Biol. 2004 Jan. 2; 335 (1): 49-56; Stijlemans et al., J Biol Chem. 2004 Jan. 9; 279 (2): 1256-61; Cortez-Retamozo et al., Cancer Res. 2004 Apr. 15; 64 (8): 2853-7; Spinelli et al., FEBS Lett. 2004 Apr. 23; 564 (1-2): 35-40; Pleschberger et al., Bioconjug. Chem. 2004 May-June; 15 (3): 664-71; Nicaise et al., Protein Sci. 2004 July; 13 (7): 1882-91; Omidfar et al., Tumour Biol. 2004 July-August; 25 (4): 179-87; Omidfar et al., Tumour Biol. 2004 September-December; 25(5-6): 296-305; Szynol et al., Antimicrob Agents Chemother. 2004 September; 48(9):3390-5; Saerens et al., J. Biol. Chem. 2004 Dec. 10; 279 (50): 51965-72; De Genst et al., J. Biol. Chem. 2004 Dec. 17; 279 (51): 53593-601; Dolk et al., Appl. Environ. Microbiol. 2005 January; 71(1): 442-50; Joosten et al., Appl Microbiol Biotechnol. 2005 January; 66(4): 384-92; Dumoulin et al., J. Mol. Biol. 2005 Feb. 25; 346 (3): 773-88; Yau et al., J Immunol Methods. 2005 February; 297 (1-2): 213-24; De Genst et al., J. Biol. Chem. 2005 Apr. 8; 280 (14): 14114-21; Huang et al., Eur. J. Hum. Genet. 2005 Apr. 13; Dolk et al., Proteins. 2005 May 15; 59 (3): 555-64; Bond et al., J. Mol. Biol. 2005 May 6; 348(3):699-709; Zarebski et al., J. Mol. Biol. 2005 Apr. 21.

In accordance with the terminology used in the above references, the variable domains present in naturally occurring heavy chain antibodies will also be referred to as “VHH domains”, in order to distinguish them from the heavy chain variable domains that are present in conventional 4-chain antibodies (which will be referred to herein as “VH domains”) and from the light chain variable domains that are present in conventional 4-chain antibodies (which will be referred to herein as “VL domains”). As mentioned in the prior art referred to above, VHH domains have a number of unique structural characteristics and functional properties which make isolated VHH domains (as well as Nanobodies based thereon, which share these structural characteristics and functional properties with the naturally occurring VHH domains) and proteins containing the same highly advantageous for use as functional antigen-binding domains or proteins. In particular, and without being limited thereto, VHH domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the VHH domains from the VH and VL domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a VH domain covalently linked to a VL domain).

In further embodiments, the antibody or antibody fragment may be multispecific (such as a bispecific, trispecific, etc. antibody) comprising at least two (such as two, three, etc.) binding sites, each directed against a different antigen or antigenic determinant.

In some embodiments, the therapeutic agent may be a dual variable domain immunoglobulin (DVD-Ig™).

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

The term antibody as used herein also encompasses “chimeric antibodies” which originate from at least two animal species. The term “chimeric antibody” or “chimeric antibodies” refers to antibodies which comprise heavy and light chain variable region sequences from one species and constant region sequences from another species, such as for example antibodies having murine heavy and light chain variable regions linked to human, canine, equine, or feline constant regions. Chimeric antibodies comprise a portion of the heavy and/or light chain that is identical to or homologous with corresponding sequences from antibodies derived from a particular species or belonging to a particular antibody class or subclass, while the remainder of the chain(s) is identical to or homologous with corresponding sequences in antibodies from another species or belonging to another antibody class or subclass, as well as fragments of such antibodies, exhibiting the desired biological activity (See e.g., U.S. Pat. No. 4,816,567; and Morrison et al., Proc. Natl. Acad. Sci. USA 81:6851-6855 (1984)). Chimeric antibodies are made through merging DNA encoding a portion, such as the Fv region, of a monoclonal antibody from one species, e.g. mouse or monkey, with the antibody-producing DNA from another species, e.g. human.

The term antibody as used herein also encompasses “fully human antibodies”. The term “human antibody” or “fully human antibody” refers to an antibody of which the encoding genetic information is of human origin. Accordingly, the term “fully human antibody” refers to antibodies having variable and constant regions derived only from human germline immunoglobulin sequences. The term “fully human antibody” is thus not to include antibodies in which CDR sequences derived from the germline of other mammalian species, such as a mouse, have been grafted onto human framework sequences. Fully human antibodies may be derived from phage human antibody libraries as described above, or they may be obtained through immunization of transgenic mice which have been engineered to replace the murine immunoglobulin encoding region as described in Lonberg and Husznar 1995 (Int. Rev. Immunol. 13 (1): 65-93). Fully human antibodies that are made using phage display are preferably produced by recombinant expression in a human cell line resulting in antibodies with a human glycosylation pattern. Non-limiting examples of fully human antibodies are HuCAL® antibodies (Morphosys). The genetic information for constructing a HuCAL® antibody is extracted from the HuCAL® antibody library (Morphosys) and introduced into human PER.C6® cells in the form of a vector (i.e., transfection). The transfected cells translate the genetic information into protein. The protein is further modified by glycosylation and the resulting antibody molecule is finally secreted by the cells into the culture medium.

The term antibody as used herein also encompasses “humanized antibodies”. The term “humanized antibody” refers to antibodies derived from non-human species whose protein sequence have been modified so as to increase their similarity to antibodies produced naturally in humans, more particularly, antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which non-human CDR sequences are introduced into human VH and VL sequences to replace the corresponding human CDR sequences.

The humanized antibody is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. A humanized antibody comprises substantially all, or at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the framework regions are those of a human immunoglobulin consensus sequence. A humanized antibody also comprises at least a portion of an immunoglobulin constant region (Fc), typically that of a human immunoglobulin.

A humanized antibody may contain both the light chain as well as at least the variable domain of a heavy chain. The antibody also may include the CHI, hinge, CH2, CH3, and CH4 regions of the heavy chain. Alternatively, a humanized antibody may only contain a humanized light chain, or a humanized heavy chain. An exemplary humanized antibody contains a humanized variable domain of a light chain and a humanized variable domain of a heavy chain.

Also, for example, humanized antibodies may be derived from conventional antibodies from the family Camelidae, in particular from the llama (e.g., Lama paccos, Lama glama or Lama vicugna), whose variable domains exhibit a high degree of amino acid sequence identity with the variable domains of human antibodies. A suitable platform for the production of such humanized antibodies is the SIMPLE Antibody™ platform (ArGEN-X) as described in WO 2011080350.

The term antibody as used herein also encompasses “primatized antibodies”. The term “primatized antibody” refers to antibodies derived from non-primate species whose protein sequence have been modified so as to increase their similarity to antibodies produced naturally in primates (e.g. macaque), more particularly, antibodies which comprise heavy and light chain variable region sequences from a non-primate species (e.g., a mouse) but in which at least a portion of the VH and/or VL sequence has been altered to be more “primate-like”, i.e., more similar to primate germline variable sequences. Primatized antibodies are structurally identical to human antibodies and reduce potential human-anti-mouse reactivity.

The term antibody as used herein also encompasses “intrabodies”. The term “intrabody” generally refers to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention or retrieval signal sequence KDEL.

A skilled person will understand that an antibody can include one or more amino acid deletions, additions and/or substitutions (e.g., conservative substitutions), insofar such alterations preserve its binding of the respective antigen. For example, mutations may be introduced into the antibody, in particular in the Fc region, to extend in vivo half-life without compromising immunogenicity as described in U.S. Pat. No. 8,323,962. An antibody may also include one or more native or artificial modifications of its constituent amino acid residues (e.g., glycosylation, etc.).

Methods for immunising animals, e.g., non-human animals such as laboratory or farm animals, using immunising antigens (such as, e.g., the herein disclosed CD80 or CD86 polypeptides) optionally fused to or covalently or non-covalently linked, bound or adsorbed to a presenting carrier, and preparation of antibody or cell reagents from immune sera is well-known per se and described in documents referred to elsewhere in this specification. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles. Following immunization, the antibody-producing cells from the animals may be isolated and used to generate monoclonal antibody-producing hybridoma cells using techniques well-known in the art.

Methods for producing recombinant antibodies or fragments thereof need a host organism or cell. The terms “host cell” and “host organism” may suitably refer to cells or organisms encompassing both prokaryotes, such as bacteria, and eukaryotes, such as yeast, fungi, protozoan, plants and animals. Contemplated as host organisms or cells for the production of antibodies include inter alia unicellular organisms, such as bacteria (e.g., E. coli), and (cultured) animal cells (e.g., mammalian cells or human cells). The advantages of producing antibodies in bacteria are amongst other the relatively safe and straightforward handling of bacterial cells and the rapid replication cycles of microorganisms. Bacteria are particularly suitable for the production of antibody fragments with a simple structure. For the production of full-length immunoglobulins or more complex antibody fragments in prokaryotic cells, the bacterial cells may be transformed with at least two nucleic acids each encoding a different portion of the antibody fragment or the immunoglobulin, e.g., the heavy chain or the light chain, as described in WO 2009021548 for full-length immunoglobulins. In the bacterial cell, the genetic information encoding the antibody is read and translated into a protein. The resulting antibodies accumulate in the periplasmic space and can be harvested upon lysis of the bacterial cells. A further separation step may be performed to purify the antibodies. WO 2009021548 describes an E. coli-based secretion system wherein the bacteria release the antibodies in the surrounding culture medium due to the introduction of a signal sequence into the antibody encoding construct. This enables the easy and convenient purification of the antibodies from the cell culture medium. An exemplary mammalian cell line that can be used for the production of antibodies is the Chinese hamster ovary (CHO) cell line. An exemplary human cell line suitable for the production of antibodies includes the PER.C6® cell line as deposited under ECAC no. 96022940 and described in WO 2000063403 or a derivative thereof. Human cell lines are particularly suitable for the production of fully human antibodies because they produce antibodies with a human glycosylation pattern.

Unless indicated otherwise, all methods, steps, techniques and manipulations that are not specifically described in detail can be performed and have been performed in a manner known per se, as will be clear to the skilled person. Reference is for example again made to standard handbooks as well as to the general background art referred to herein and to the further references cited therein.

Examples of antibodies capable of binding to RHBG, which may be suitable for measuring the RHBG protein, more particularly human RHBG protein, by methods appropriate for the respective antibodies (information about the suitability of a given antibody for a given antigen detection technique or method is readily available from the vendor of the antibody), include without limitation those available from the following vendors (“#” stands for catalogue number): Abcam (Cambridge, UK) (# ab136658, # ab106801, goat polyclonals, # ab102591, rabbit polyclonal), Sigma-Aldrich (St. Louis, Mo., US) (# HPA048489, # SAB2105731, rabbit polyclonals), ThermoFisher Scientific (Waltham, Mass., US) (# PA5-26978, # PA5-43367, rabbit polyclonals), OriGene Technologies (Rockville, Md., US) (# TA338416, # TA338417, # AP53655PU-N, # ARP49502-P050, # ARP49503-P050, # NBP1-59847, # NBP1-69483, rabbit polyclonals), Santa Cruz (# sc-398816, mouse monoclonal clone B-9, IgG1 (kappa light chain), residues 337-408).

Numerous antibodies binding to other markers, peptides, polypeptides and proteins described herein, such as antibodies to RHAG or RHCG, are also commercially available from a variety of vendors. This information can be obtained from the respective vendors, and is also conveniently catalogued and can be queried in publically available databases, such as the GeneCards® database maintained by the Weizmann Institute (www.genecards.org), field “Antibody products”.

In some embodiments, binding agents as taught herein may comprise a detectable label. The term “label” refers to any atom, molecule, moiety or biomolecule that may be used to provide a detectable and preferably quantifiable read-out or property, and that may be attached to or made part of an entity of interest, such as a binding agent. Labels may be suitably detectable by for example mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.) alone or in combination with moieties that may suppress or shift emission spectra by fluorescence resonance energy transfer (FRET).

In some embodiments, binding agents may be provided with a tag that permits detection with another agent (e.g., with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc.

The biomarker—binding agent conjugate may be associated with or attached to a detection agent to facilitate detection. Examples of detection agents include, but are not limited to, luminescent labels; colourimetric labels, such as dyes; fluorescent labels; or chemical labels, such as electroactive agents (e.g., ferrocyanide); enzymes; radioactive labels; or radiofrequency labels. The detection agent may be a particle. Examples of such particles include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles may be colloidal gold particles.

In particular embodiments, the means to detect said at least one ammonium transporter, such as preferably at least one ammonium transporter selected from the group consisting of RHBG, RHAG, RHCG, and combinations thereof, such as particularly preferably RHBG may be primers or probes selectively detecting the expression of nucleic acids encoding said at least one ammonium transporter, e.g., mRNA encoding said at least one ammonium transporter (e.g., by quantitative RT-PCR). In more particular embodiments, RHBG may be detected using a primer pair comprising a forward primer as set forth in SEQ ID NO: 3 (5′-CTGTGAGTGCAGCATTGAAG-3′) and a reverse primer as set forth in SEQ ID NO: 4 (5′-CTACCATTCAGACCTCTTCGC-3′) and/or a probe/56-FAM/CCATCTTCC/ZEN/TGTGGATCTTCTGGCC/31ABkFQ/(SEQ ID NO: 5). In other embodiments, RHBG may be detected using a primer pair comprising a forward primer as set forth in SEQ ID NO: 9 (5′-CCTCAAGTGAAATGATGCTG-3′) and a reverse primer as set forth in SEQ ID NO: 10 (5′-ATTTTGATTCAAGGATGGGC-3′).

As used herein, the term “primer” or “amplification primer” refers to a single-stranded oligonucleotide, more preferably to a DNA oligonucleotide, which is (or part of which is) complementary or sufficiently complementary to a sequence comprised in a nucleic acid to be amplified by polymerase-based amplification process, e.g., PCR, such that the primer can hybridise (anneal) with said sequence and can act as a point of initiation of synthesis of a primer extension product in the presence of nucleotides and a nucleic acid polymerase, e.g., DNA polymerase. A primer needs to be sufficiently long to prime the synthesis of an extension product. A typical primer may thus be at least 10 nucleotides in length, e.g., at least 11, at least 12, at least 13 or at least 14 nucleotides in length, preferably at least 15 nucleotides in length, e.g., at least 16, at least 17, at least 18 or at least 19 nucleotides in length, more preferably at least 20 nucleotides in length. Further preferred primers are between about 10 and about 40 nucleotides in length, more preferably between about 15 and about 30 nucleotides in length, most preferably between about 18 and about 26 nucleotides long.

The term “primer pair” or “amplification primer pair” refers to a combination of two primers which are suited for amplification of a target nucleic acid region (amplicon) from within a nucleic acid of interest by a polymerase-based amplification process, e.g., PCR. The ability to amplify an amplicon from within the nucleic acid of interest using a primer pair designed to specifically hybridise within the nucleic acid indicates the presence (and optionally quantity) of the nucleic acid in the polymerase-based amplification reaction.

The term “oligonucleotide” as used herein refers to a nucleic acid (including nucleic acid analogues and mimetics) oligomer or polymer as defined herein. Preferably, an oligonucleotide is (substantially) single-stranded. Oligonucleotides as intended herein may be preferably between about 10 and about 100 nucleoside units (i.e., nucleotides or nucleotide analogues) in length, preferably between about 15 and about 50, more preferably between about 15 and about 40, also preferably between about 20 and about 30.

In particular embodiments, oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, enhanced cellular uptake, increased stability in the presence of nucleases and increased hybridization affinity, increased tolerance for mismatches, etc. Further, oligonucleotides as intended herein may be configured to not activate RNAse H, accordance with known techniques (see, e.g., U.S. Pat. No. 5,149,797).

As used herein, the term “probe” refers to an oligonucleotide, more preferably to a DNA oligonucleotide, which (or part of which) is complementary or sufficiently complementary as defined herein to a sequence comprised in a nucleic acid to be detected by the probe, such that the probe can hybridise (anneal) with said sequence. In particular, probes as intended herewith can hybridise (anneal) with a primer extension product produced by the polymerase-based amplification.

In certain embodiments, probes as taught herein may be defined as configured to hybridise (anneal) within certain recited nucleic acid sequences. In this context, the phrase “hybridise within a nucleic acid” or “hybridise within a nucleic acid sequence” is intended to mean that the probe may anneal to the whole of the recited nucleic acid sequence, or only to a portion of the recited nucleic acid sequence, but does not anneal to sequences adjacent to but outside of the recited nucleic acid sequence.

Probes may be ideally less than or equal to about 50 nucleotides in length, for example less than or equal to about 40, about 30, about 20, or less than about 10 nucleotides in length, e.g., between 10 and 30 or between 15 and 25 nucleotides in length.

A probe comprises an oligonucleotide sequence which effects the hybridisation (annealing) of the probe with a sequence comprised in a nucleic acid to be detected by the probe. In certain embodiments, a probe does not contain any further oligonucleotide sequence(s). In certain other embodiments, a probe may contain—besides the oligonucleotide sequence which effects the hybridisation of the probe with a sequence comprised in a nucleic acid to be detected by the probe—additional oligonucleotide sequence(s) serving other useful purpose(s). For example but without limitation, such additional oligonucleotide sequence(s) may provide linker sequences allowing to couple a probe with another moiety or moieties, e.g., label(s) or reporter moiety, e.g., a radioactive isotope (e.g., ³²P, ³³P), ligand, chemiluminescent agent, fluorophore (e.g., fluorescein, tetrachloro-fluorescein, TAMRA, ROX, Cy3, Cy3.5, Cy5, Cy5.5, Texas Red, etc.), vitamin (e.g., biotin), steroid (e.g., digoxin), enzyme (e.g., HRP, AP, etc.), etc., or may provide sequences ensuring a certain conformation of a probe, etc.; various options are available to a skilled reader.

By means of example and not limitation, when a probe forms a molecular beacon as known in the art, mutually complementary oligonucleotide extensions are provided at the 5′ and 3′ ends of the probe, one of the oligonucleotide extensions linked to a fluorophore (e.g., fluorescein, carboxyfluorescein (FAM), tetrachloro-fluorescein, TAMRA, ROX, Cy3, Cy3.5, Cy5, Cy5.5, Texas Red, etc.) and the other one to a quencher (e.g. ZEN™ internal quencher, 3′ Iowa Black Black® FQ quencher) capable of quenching the fluorescent emission of the fluorophore. When the probe is not annealed to the nucleic acid to be detected, the mutually complementary oligonucleotide extensions will form a hairpin structure, whereby the quencher is brought into proximity of the fluorophore and quenches the fluorophore's signal. Conversely, when the probe is annealed to the nucleic acid to be detected, the hairpin structure cannot formed, the quencher is not in proximity of the fluorophore and does not quench the fluorophore's signal, which signal is therefore detectable.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require first to determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need first to convert the readouts to absolute values of the respective parameters.

The terms “quantity” and “expression level” of said at least one ammonium transporter, such as preferably at least one ammonium transporter selected from the group consisting of RHBG, RHAG, RHCG, and combinations thereof, such as particularly preferably RHBG, are used interchangeably in this specification to refer to the absolute and/or relative quantification, concentration level or amount of any such product in a sample.

Distinct reference values may represent the diagnosis of a proliferative disease vs. the absence of a proliferative disease (such as, e.g., healthy or recovered from a proliferative disease). In another example, distinct reference values may represent the diagnosis of a proliferative disease of varying severity.

Alternatively, distinct reference values may represent the prediction of a risk (e.g., an abnormally elevated risk) of developing a proliferative disease vs. the prediction of no or normal risk of developing a proliferative disease. In another example, distinct reference values may represent predictions of differing degrees of risk of developing a proliferative disease.

In yet another example, distinct reference values may represent the need of a subject for a therapeutic or prophylactic treatment of a proliferative disease vs. no need of a subject for a therapeutic or prophylactic treatment of a proliferative disease. In a further example, distinct reference values may represent various urgencies of the need for a therapeutic or prophylactic treatment of a proliferative disease or the need for a specific type of therapeutic or prophylactic treatment of a proliferative disease.

Moreover, distinct reference values may represent a good prognosis for a proliferative disease vs. a poor prognosis for a proliferative disease. In a further example, distinct reference values may represent varyingly favourable or unfavourable prognoses for a proliferative disease.

Such comparison may generally include any means to determine the presence or absence of at least one difference and optionally of the size of such difference between values or profiles being compared. A comparison may include a visual inspection, an arithmetical or statistical comparison of measurements. Such statistical comparisons include, but are not limited to, applying an algorithm. If the values or biomarker profiles comprise at least one standard, the comparison to determine a difference in said values or biomarker profiles may also include measurements of these standards, such that measurements of the biomarker are correlated to measurements of the internal standards.

Reference values for the quantity or expression level of said at least one ammonium transporter, such as preferably at least one ammonium transporter selected from the group consisting of RHBG, RHAG, RHCG, and combinations thereof, such as particularly preferably RHBG may be established according to known procedures previously employed for other biomarkers. For example, a reference value of the amount of said at least one ammonium transporter for a particular diagnosis, prediction, prognosis and/or monitoring of a proliferative disease or for the determination whether a subject is in need of therapeutic or prophylactic treatment as taught herein may be established by determining the quantity or expression level of said at least one ammonium transporter in sample(s) from one individual or from a population of individuals characterised by said particular diagnosis, prediction, prognosis and/or monitoring of said disease or condition or for the determination whether a subject is in need of therapeutic or prophylactic treatment of said disease or condition. Such population may comprise without limitation ≥2, ≥10, ≥100, or even several hundred individuals or more. In the context of predictive methods or uses, the status of an individual or population of individuals as to the disease progression of a proliferative disease, or disease severity of a proliferative disease, or responsiveness of a proliferative disease to a proliferative disease therapy may not be known at the time of sampling said individual or population of individuals, but will become known later on, such that the reference value generated on the basis of said individual or population of individuals can then be allocated to the particular prediction of disease progression of a proliferative disease, or of disease severity of a proliferative disease, or of responsiveness of a proliferative disease to a proliferative disease therapy, as observed in said individual or population of individuals.

Hence, by means of an illustrative example, reference values of the quantity or expression level of said at least one ammonium transporter, such as preferably at least one ammonium transporter selected from the group consisting of RHBG, RHAG, RHCG, and combinations thereof, such as particularly preferably RHBG for the diagnosis of a proliferative disease vs. no such disease or condition may be established by determining the quantity or expression level of said at least one ammonium transporter expression in sample(s) from one individual or from a population of individuals diagnosed (e.g., based on other adequately conclusive means, such as, for example, clinical signs and symptoms, imaging, etc.) as, respectively, having or not having a proliferative disease.

Measuring the quantity or expression level of said at least one ammonium transporter, such as preferably at least one ammonium transporter selected from the group consisting of RHBG, RHAG, RHCG, and combinations thereof, such as particularly preferably RHBG for the same patient at different time points may in such a case thus enable the continuous monitoring of the status of the patient and may lead to prediction of worsening or improvement of the patient's condition with regard to a given disease or condition as taught herein. Tools such as the kits described herein below can be developed to ensure this type of monitoring. One or more reference values or ranges for said at least one ammonium transporter quantities or expression levels linked to the development of a proliferative disease can, e.g., be determined beforehand or during the monitoring process over a certain period of time in said subject. Alternatively, these reference values or ranges can be established through data sets of several patients with highly similar disease phenotypes, e.g., from subjects not developing a proliferative disease. A sudden deviation of the levels of said at least one ammonium transporter from said reference value or range can predict the worsening of the condition of the patient (e.g., at home or in the clinic) before the (often severe) symptoms actually can be felt or observed.

In an embodiment, reference value(s) as intended herein may convey absolute quantities of the biomarker as intended herein. In another embodiment, the quantity of the biomarker in a sample from a tested subject may be determined directly relative to the reference value (e.g., in terms of increase or decrease, or fold-increase or fold-decrease). Advantageously, this may allow the comparison of the quantity or expression level of the biomarker in the sample from the subject with the reference value (in other words to measure the relative quantity of the biomarker in the sample from the subject vis-à-vis the reference value) without the need first to determine the respective absolute quantities of the biomarker.

As explained, the present methods, uses, or products may involve finding a deviation or no deviation between the quantity or expression level of said at least one ammonium transporter, such as preferably at least one ammonium transporter selected from the group consisting of RHBG, RHAG, RHCG, and combinations thereof, such as particularly preferably RHBG as taught herein measured in a sample from a subject and a given reference value.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given biomarker, e.g., an optimal cut-off value of the normalised surface area of the immuno-staining for said at least one ammonium transporter protein, such as preferably at least one ammonium transporter protein selected from the group consisting of RHBG, RHAG, RHCG, and combinations thereof, such as particularly preferably RHBG protein (see above), for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

The present methods, uses, or products may further involve attributing any finding of a deviation or no deviation between the quantity or expression level of said at least one ammonium transporter, such as preferably at least one ammonium transporter selected from the group consisting of RHBG, RHAG, RHCG, and combinations thereof, such as particularly preferably RHBG as taught herein measured in a sample from a subject and a given reference value to the presence or absence of a proliferative disease, a particular prediction of disease progression of a proliferative disease, or of disease severity of a proliferative disease or a particular prognosis of a proliferative disease.

In the methods provided herein the observation of a deviation between the quantity or expression level of said at least one ammonium transporter in a tissue sample from a subject and a reference value can lead to the conclusion that the diagnosis, prediction and/or prognosis of said proliferative disease in said subject is different from that represented by said reference value. Similarly, when no deviation is found between the quantity or expression level of said at least one ammonium transporter in a tissue sample from a subject and a reference value, the absence of such deviation can lead to the conclusion that the diagnosis, prediction and/or prognosis of said proliferative disease in said subject is substantially the same as that represented by said reference value.

In particular embodiments, the reference value as used in the methods according to the invention is determined from a tissue not affected by the proliferative disease, such as wherein said reference value is determined from healthy tissue. The quantity or expression level of said at least one ammonium transporter, such as preferably at least one ammonium transporter selected from the group consisting of RHBG, RHAG, RHCG, and combinations thereof, such as particularly preferably RHBG in a tissue sample from a subject (preferably but without limitation, subject with a proliferative disease) may be elevated compared to (i.e., relative to) a reference value representing the quantity or expression level of said at least one ammonium transporter in a tissue not affected by the proliferative disease, such as a healthy tissue sample. The so-elevated quantity or expression level may allow for the diagnosis of a proliferative disease in the subject.

In particular embodiments, the quantity or expression level of said at least one ammonium transporter, such as preferably at least one ammonium transporter selected from the group consisting of RHBG, RHAG, RHCG, and combinations thereof, such as particularly preferably RHBG in a tissue sample from a subject (preferably but without limitation, subject with a proliferative disease) may be elevated compared to (i.e., relative to) a reference value representing the prediction of normal (not elevated) probability of progression of a proliferative disease (or of comparatively more rapid progression of the proliferative disease compared to average) within a given time period. The so-elevated quantity or expression level may allow for the prediction that the subject has an elevated risk of progression of a proliferative disease (or of comparatively more rapid progression of the proliferative disease compared to average) within the given time period. In other preferred embodiments, the quantity or expression level of said at least one ammonium transporter, such as preferably at least one ammonium transporter selected from the group consisting of RHBG, RHAG, RHCG, and combinations thereof, such as particularly preferably RHBG in a tissue sample from a subject may be elevated compared to (i.e., relative to) a reference value representing a given degree of probability of progression of a proliferative disease (or of comparatively more rapid progression of the proliferative disease compared to average) (e.g., “low probability” or “moderate probability”) within a given time period. The so-elevated quantity or expression level may allow for the prediction that the subject has a comparatively greater probability of progression of a proliferative disease (or of comparatively more rapid progression of the proliferative disease compared to average) (e.g., “moderate probability” vs. “low probability”, or “high probability” vs. “moderate probability” or “low probability”) within the given time period.

In particular embodiments, the reference value as used in the methods according to the invention is determined from healthy breast tissue or basal breast cancer tissue. The quantity or expression level of said at least one ammonium transporter, such as preferably at least one ammonium transporter selected from the group consisting of RHBG, RHAG, RHCG, and combinations thereof, such as particularly preferably RHBG in a tissue sample obtained from a breast tumour from a subject may be elevated compared to (i.e., relative to) a reference value representing the quantity or expression level of said at least one ammonium transporter in healthy breast tissue or in a basal breast cancer tissue. The so-elevated quantity or expression level may allow for the diagnosis of luminal breast cancer in the subject. In further particular embodiments, the quantity or expression level of said at least one ammonium transporter, such as preferably at least one ammonium transporter selected from the group consisting of RHBG, RHAG, RHCG, and combinations thereof, such as particularly preferably RHBG in a tissue sample from a breast tumour from a subject may be elevated compared to (i.e., relative to) a reference value representing the prediction of the development of basal breast cancer within a given time period. The so-elevated quantity or expression level may allow for the prediction that the subject will display a less severe or aggressive type of breast cancer within the given time period, preferably luminal breast cancer. In other preferred embodiments, the quantity or expression level of said at least one ammonium transporter, such as preferably at least one ammonium transporter selected from the group consisting of RHBG, RHAG, RHCG, and combinations thereof, such as particularly preferably RHBG in a tissue sample from a breast tumour from a subject may be elevated compared to (i.e., relative to) a reference value representing the prediction of the development of basal breast tumour (“high severity”) within a given time period. The so-elevated quantity or expression level may allow for the prediction that the subject will display comparatively lower degree of severity of breast tumour (e.g., “low severity” or. “moderate severity” vs. “high severity”) within the given time period.

In particular embodiments, any one of the methods as taught herein may allow to differentiate between luminal breast cancer and basal breast cancer.

In particular embodiments, an increase of at least 1.5-fold, i.e., there is at least a 50% increase, of quantity of said at least one ammonium transporter, such as preferably at least one ammonium transporter selected from the group consisting of RHBG, RHAG, RHCG, and combinations thereof, such as particularly preferably RHBG or expression level, is indicative for the presence of a proliferative disease, preferably at least a 2-fold increase, more preferably at least a 3-fold increase.

A further aspect of the invention relates to a kit for diagnosing, predicting, prognosing and/or monitoring a proliferative disease in a subject, the kit comprising:
(a) means for measuring the quantity or expression level of said at least one ammonium transporter, such as preferably at least one ammonium transporter selected from the group consisting of RHBG, RHAG, RHCG, and combinations thereof, such as particularly preferably RHBG in a tissue sample from a subject; and
(b) a reference value of the quantity or expression level of said at least one ammonium transporter or means for establishing said reference value, wherein said reference value represents a known diagnosis, prediction and/or prognosis of the proliferative disease, such as wherein said reference value corresponds to the quantity or expression level of said at least one ammonium transporter in a tissue not affected by the proliferative disease, such as in a healthy tissue, or wherein said reference value corresponds to the quantity or expression level of said at least one ammonium transporter in a tissue affected by the proliferative disease.

The kit for diagnosing, predicting, prognosing and/or monitoring a proliferative disease in a subject may further comprise ready-to use substrate solutions, wash solutions, dilution buffers and instructions. The diagnostic kit may also comprise positive and/or negative control samples.

Preferably, the instructions included in the diagnostic kit are unambiguous, concise and comprehensible to those skilled in the art. The instructions typically provide information on kit contents, how to collect the tissue sample, methodology, experimental read-outs and interpretation thereof and cautions and warnings.

The terms “kit of parts” and “kit” as used throughout this specification refer to a product containing components necessary for carrying out the specified methods (e.g., methods for the diagnosis, prediction, prognosis and/or monitoring of a proliferative disease in a subject or for determining whether a subject is in need of therapeutic or prophylactic treatment of a proliferative disease as taught herein), packed so as to allow their transport and storage.

Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridisation probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

The means for measuring the quantity or expression level of said at least one ammonium transporter, such as preferably at least one ammonium transporter selected from the group consisting of RHBG, RHAG, RHCG, and combinations thereof, such as particularly preferably RHBG in a tissue sample from a subject may comprise binding agents as discussed elsewhere in this specification and/or carriers which allow visualization and/or a qualitative read-out of the measurement, for example, by spectrophotometry. Optionally, these carriers allow for cascade testing. Non-limiting examples of carriers are translucent microtiter plates, translucent stripwells or translucent tubes.

Moreover, the present invention also relates to a kit for diagnosing, predicting, prognosing and/or monitoring luminal breast cancer in a subject, the kit comprising:
(i) means for measuring the quantity or expression level of said at least one ammonium transporter, such as preferably at least one ammonium transporter selected from the group consisting of RHBG, RHAG, RHCG, and combinations thereof, such as particularly preferably RHBG in a tissue sample obtained from a breast tumour from a subject; and
(ii) a reference value of the quantity of said at least one ammonium transporter or means for establishing said reference value, wherein said reference value represents the quantity or expression level of said at least one ammonium transporter in a healthy breast tissue or in a basal breast cancer tissue.

In particular embodiments, the kit according to the invention is used for the diagnosis, prediction, prognosis and/or monitoring of luminal breast cancer in a subject.

The commercial available antibodies for human RHBG that are currently on the market are not very effective when used in methods for detecting human RHBG by immunohistochemistry and protein immunoblotting (e.g., Western blot, dot blot). Without wishing to be bound by any hypothesis, this may at least in part be due to some antibodies being directed against an incorrect C-terminal amino acid sequence as annotated under NCBI Genbank (http://www.ncbi.nlm.nih.gov/) accession number NM_001256395.1, NM_001256396.1, NM_020407.4, NP_001243324.1, NP_001243325.1 or NP_065140.3. In case of antibodies directed against the N-terminus of RHBG, this may potentially be due to the fact that the N-terminal portion of RHBG is typically glycosylated in cells, and said glycosylation might impair antibody recognition and binding.

The present inventors developed a new antibody capable of binding human RHBG with high affinity, which antibody performs adequately in among others immunohistochemistry and protein immunoblotting. This antibody was designed against a target antigen of RHBG with an amino acid sequence according to SEQ ID NO:2 (NCBI Genbank (http://www.ncbi.nlm.nih.gov/) accession number Q9H310.2). More particularly, the antibody was designed to recognize the C-terminal epitope with the ‘correct’ amino acid sequence DSPPRLPALRGPSS (SEQ ID NO: 8).

In view hereof, another aspect of the invention relates to an antibody or a functional fragment thereof characterised in that the antibody or the functional fragment thereof binds epitope DSPPRLPALRGPSS of human RHBG as set out in SEQ ID NO: 15.

In particular embodiments, the uses, methods, and products as taught in the application for diagnosis, prediction, prognosis and/or monitoring of proliferative diseases may employ the antibody, more particularly, as an agent capable of specifically binding to said at least one ammonium transporter, such as preferably at least one ammonium transporter selected from the group consisting of RHBG, RHAG, RHCG, and combinations thereof, such as particularly preferably RHBG

Given the differential expression of said at least one ammonium transporter, such as preferably at least one ammonium transporter selected from the group consisting of RHBG, RHAG, RHCG, and combinations thereof, such as particularly preferably RHBG in proliferative diseases, said at least one ammonium transporter, such as preferably at least one ammonium transporter selected from the group consisting of RHBG, RHAG, RHCG, and combinations thereof, such as particularly preferably RHBG represents a valuable target for therapies in these diseases.

Without wishing to be bound by any theory, the present inventors hypothesise that in certain instances said at least one ammonium transporter, such as preferably at least one ammonium transporter selected from the group consisting of RHBG, RHAG, RHCG, and combinations thereof, such as particularly preferably RHBG may potentially plays a role in removing ammonia and/or ammonium from cancer cells, which display an altered metabolism and are for their energy production dependent on glutaminolysis, which produces ammonia and/or ammonium as a side product. In such instances, interfering with removal of ammonia and/or ammonium mediated by said at least one ammonium transporter, such as preferably at least one ammonium transporter selected from the group consisting of RHBG, RHAG, RHCG, and combinations thereof, such as particularly preferably RHBG from the cell can be useful to fight the cancer.

Alternatively or in addition, the present inventors hypothesise the potential occurrence of an ammonium shuttle between glutamine-consuming cells and glutamine-synthesizing cells in the tumor environment. This takes into account the fact that said at least one ammonium transporter, such as preferably at least one ammonium transporter selected from the group consisting of RHBG, RHAG, RHCG, and combinations thereof, such as particularly preferably RHBG is bidirectional and can facilitate ammonium transport into as well as out of cells across or along an existing ammonium concentration gradient, wherein a given direction of ammonium transport can be favored based on the cellular and environment contexts. The cellular context of tumors is heterogeneous, containing neoplastic as well as non-neoplastic cells, such as stromal cells. Between such cells, for example between cancer cells and stromal cells, a coupling of cells with different and complementary metabolic factories may arise. Hence, cells actively consuming glutamine via glutaminase (such as cancer cells) will release ammonium, which can be favored if said at least one ammonium transporter, such as preferably at least one ammonium transporter selected from the group consisting of RHBG, RHAG, RHCG, and combinations thereof, such as particularly preferably RHBG is expressed. Other cells (such as cells in the stroma) expressing glutamine synthetase could play a role in detoxification of the ammonium by converting it into glutamine, which will in turn release glutamine to be used by the glutamine-consuming cells. Detoxifying cells expressing said at least one ammonium transporter, such as preferably at least one ammonium transporter selected from the group consisting of RHBG, RHAG, RHCG, and combinations thereof, such as particularly preferably RHBG, which will transport the ammonium into the cells to ‘feed’ the glutamine synthase, may therefore also be targeted by modulators of said at least one ammonium transporter, such as preferably at least one ammonium transporter selected from the group consisting of RHBG, RHAG, RHCG, and combinations thereof, such as particularly preferably RHBG, such as inhibitors, leading to impaired detoxification of the ammonium in the tumor environment as a whole.

In view of the above, a further aspect of the invention relates to a therapeutic or prophylactic agent for use as a medicament.

In particular embodiments, the therapeutic or prophylactic agent according to the invention is used as a medicament in the treatment of a proliferative disease, wherein said therapeutic agent is capable of modulating expression or activity of said at least one ammonium transporter, such as preferably at least one ammonium transporter selected from the group consisting of RHBG, RHAG, RHCG, and combinations thereof, such as particularly preferably RHBG.

In particular embodiments, the therapeutic or prophylactic agent according to the invention is used as a medicament in the treatment of a proliferative disease, wherein said therapeutic agent is capable of inhibiting expression or activity of said at least one ammonium transporter, such as preferably at least one ammonium transporter selected from the group consisting of RHBG, RHAG, RHCG, and combinations thereof, such as particularly preferably RHBG.

In particular embodiments, the therapeutic or prophylactic agent according to the invention is used as a medicament in the treatment of a proliferative disease, wherein said therapeutic agent is capable of increasing expression or activity of said at least one ammonium transporter, such as preferably at least one ammonium transporter selected from the group consisting of RHBG, RHAG, RHCG, and combinations thereof, such as particularly preferably RHBG.

In a related aspect, the invention provides the use of a therapeutic or prophylactic agent in the treatment of a proliferative disease, wherein said therapeutic agent is capable of modulating expression or activity of said at least one ammonium transporter, such as preferably at least one ammonium transporter selected from the group consisting of RHBG, RHAG, RHCG, and combinations thereof, such as particularly preferably RHBG.

In a related aspect, the invention provides the use of a therapeutic or prophylactic agent in the treatment of a proliferative disease, wherein said therapeutic agent is capable of inhibiting expression or activity of said at least one ammonium transporter, such as preferably at least one ammonium transporter selected from the group consisting of RHBG, RHAG, RHCG, and combinations thereof, such as particularly preferably RHBG.

In a related aspect, the invention provides the use of a therapeutic or prophylactic agent in the treatment of a proliferative disease, wherein said therapeutic agent is capable of increasing expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides the use of a therapeutic or prophylactic agent for the manufacture of a medicament for use in treatment of a proliferative disease, wherein said therapeutic agent is capable of modulating expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides the use of a therapeutic or prophylactic agent for the manufacture of a medicament for use in treatment of a proliferative disease, wherein said therapeutic agent is capable of inhibiting expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides the use of a therapeutic or prophylactic agent for the manufacture of a medicament for use in treatment of a proliferative disease, wherein said therapeutic agent is capable of increasing expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides a method of treating a proliferative disease in a subject, comprising administering to the subject a therapeutically or prophylactically amount of a therapeutic or prophylactic agent capable of modulating expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides a method of treating a proliferative disease in a subject, comprising administering to the subject a therapeutically or prophylactically amount of a therapeutic or prophylactic agent capable of inhibiting expression or activity of said at least one ammonium transporter, such as preferably at least one ammonium transporter selected from the group consisting of RHBG, RHAG, RHCG, and combinations thereof, such as particularly preferably RHBG.

In a further related aspect, the invention provides a method of treating a proliferative disease in a subject, comprising administering to the subject a therapeutically or prophylactically amount of a therapeutic or prophylactic agent capable of increasing expression or activity of said at least one ammonium transporter, such as preferably at least one ammonium transporter selected from the group consisting of RHBG, RHAG, RHCG, and combinations thereof, such as particularly preferably RHBG.

In particular embodiments, the therapeutic or prophylactic agent according to the invention modulates ammonia and/or ammonium transport mediated by said at least one ammonium transporter, such as preferably at least one ammonium transporter selected from the group consisting of RHBG, RHAG, RHCG, and combinations thereof, such as particularly preferably RHBG across the cell membrane and/or across intracellular membranes, preferably at least ammonia and/or ammonium transport mediated by said at least one ammonium transporter across the cell membrane.

In particular embodiments, the therapeutic or prophylactic agent according to the invention interferes with ammonia and/or ammonium transport mediated by said at least one ammonium transporter, such as preferably at least one ammonium transporter selected from the group consisting of RHBG, RHAG, RHCG, and combinations thereof, such as particularly preferably RHBG across the cell membrane and/or across intracellular membranes, preferably at least ammonia and/or ammonium transport mediated by said at least one ammonium transporter across the cell membrane.

In particular embodiments, the therapeutic or prophylactic agent according to the invention increases ammonia and/or ammonium transport mediated by said at least one ammonium transporter, such as preferably at least one ammonium transporter selected from the group consisting of RHBG, RHAG, RHCG, and combinations thereof, such as particularly preferably RHBG across the cell membrane and/or across intracellular membranes, preferably at least ammonia and/or ammonium transport mediated by said at least one ammonium transporter across the cell membrane.

The terms “ammonia” and “ammonium” as used herein refer to the ammonia (NH₃) and/or ammonium (NH₄) that is added to a cell by cellular uptake and to the ammonia (NH₃) and/or ammonium (NH₄) which is produced by the cell itself, for example during metabolism of amino acids, especially by glutaminolysis. Accordingly, the phrase “ammonia transport across the cell membrane” mediated by said at least one ammonium transporter as used herein refers to both the ammonia and/or ammonium transport through the transporter from outside to inside a cell as well as to the ammonia and/or ammonium transport through the transporter from inside to outside a cell, preferably from inside to outside a cell.

The phrase “ammonia transport across intracellular membranes” mediated by said at least one ammonium transporter as used herein refers to both the ammonia and/or ammonium transport through the transporter from outside to inside of a membrane-enclosed cellular organelle (such as, e.g., nucleus, mitochondrion, vacuole, endosome, endoplasmic reticulum, Golgi apparatus, or lysosome, provided the transporter is expressed in the membrane enwrapping such organelle) as well as to the ammonia and/or ammonium transport through the transporter from inside to outside of a membrane-enclosed cellular organelle (such as, e.g., nucleus, mitochondrion, vacuole, endosome, endoplasmic reticulum, Golgi apparatus, or lysosome, provided the transporter is expressed in the membrane enwrapping such organelle). The term “cell” as used herein, may refer to any type of cell, present in any type of tissue and/or organ, and present in any subject as defined herein. In particular embodiments, the cell is a cancer cell, more preferably a breast cancer cell, even more preferably, a luminal breast cancer cell.

The term “therapeutic or prophylactic agent” is used interchangeably with “drug” or “medicinal product”, and refers to an agent as taught herein used in the treatment, cure, prevention, or diagnosis of a disease.

The reference to the “activity” as used herein, is to be interpreted broadly and may generally encompass any one or more aspects of the biological activity of the target at any level (e.g., molecular, cellular and/or physiological), such as without limitation any one or more aspects of its biochemical activity, enzymatic activity, signalling activity, interaction activity, ligand activity, receptor activity or structural activity, e.g., within a cell, tissue, organ or an organism. By means of an example and not limitation, reference to the activity of the at least one ammonium transporter, such as preferably at least one ammonium transporter selected from the group consisting of RHBG, RHAG, RHCG, and combinations thereof, such as particularly preferably RHBG protein may particularly denote the activity of the polypeptide as ammonia transporter, i.e., its ability to transport ammonia across the cell membrane.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, expression or activity of said at least one ammonium transporter, such as preferably at least one ammonium transporter selected from the group consisting of RHBG, RHAG, RHCG, and combinations thereof, such as particularly preferably RHBG may be modulated without substantially altering other (unintended, undesired) phenotypic aspect(s).

The term “inhibit” as used herein is intended to be synonymous with terms such as “decrease”, “reduce”, “diminish”, “interfere”, “disrupt”, or “disturb”, and denotes a qualitative or quantitative decrease of expression and/or activity of said at least one ammonium transporter, such as preferably at least one ammonium transporter selected from the group consisting of RHBG, RHAG, RHCG, and combinations thereof, such as particularly preferably RHBG that is being interfered with. The term encompasses any extent of such interference. For example, the interference may encompass a decrease (in particular statistically significant decrease) of at least about 10%, e.g., of at least about 20%, of at least about 30%, e.g., of at least about 40%, of at least about 50%, e.g., of at least about 60%, of at least about 70%, e.g., of at least about 80%, of at least about 90%, e.g., of at least about 95%, such as of at least about 96%, 97%, 98%, 99% or even of 100%, compared to a reference situation without said interference.

The term “increase” as used herein is intended to be synonymous with terms such as “upregulate”, “enhance”, “stimulate”, or “activate”, and denotes a qualitative or quantitative increase of expression and/or activity of said at least one ammonium transporter, such as preferably at least one ammonium transporter selected from the group consisting of RHBG, RHAG, RHCG, and combinations thereof, such as particularly preferably RHBG that is being increased. The term encompasses any extent of such increase. For example, the term may encompass an increase (in particular statistically significant increase) of at least about 10%, e.g., of at least about 20%, of at least about 30%, of at least about 40%, of at least about 50%, of at least about 60%, of at least about 70%, of at least about 80%, of at least about 90%, of at least about 100% or more, including, for example at least 2-fold, at least 3-fold, at least 4-fold, at least 5-fold, at least 10-fold increase or greater as compared to a reference level without said increase.

Methods of measuring the decrease or increase of expression and/or activity of said at least one ammonium transporter, such as preferably at least one ammonium transporter selected from the group consisting of RHBG, RHAG, RHCG, and combinations thereof, such as particularly preferably RHBG are known by the skilled person. For example, the decrease or increase of activity of said at least one ammonium transporter might be reflected in a decrease or increase of ammonia transport mediated by said at least one ammonium transporter across the cell membrane. The decrease or increase of ammonia transport mediated by said at least one ammonium transporter across the cell membrane may be evaluated using stopped-flow spectrofluorimetry and the 2′,7′-bis-(2-carboxyethyl)-5(6)-carboxyfluorescein (BCECF) pH-sensitive probe to follow alterations in intracellular pH (pHᵢ) related to ammonium transport, substantially as described by Zidi-Yahiaoui et al. (supra). Ammonium transport via said at least one ammonium transporter can further be evaluated by assays to determine apparent methylammonium permeability, substantially as described in Zidi-Yahiaoui et al. or Handlogten et al. (supra).

In particular embodiments, the proliferative disease is characterised by (over)expression of said at least one ammonium transporter, such as preferably at least one ammonium transporter selected from the group consisting of RHBG, RHAG, RHCG, and combinations thereof, such as particularly preferably RHBG.

In preferred embodiments, the proliferative disease is cancer, more particularly breast cancer, even more particularly luminal breast cancer.

In particular embodiments, the therapeutic or prophylactic agent is capable of interacting with the gene RNA, preferably mRNA, encoding said at least one ammonium transporter, such as preferably at least one ammonium transporter selected from the group consisting of RHBG, RHAG, RHCG, and combinations thereof, such as particularly preferably RHBG, or the agent is capable of interacting with said at least one ammonium transporter protein, such as preferably at least one ammonium transporter protein selected from the group consisting of RHBG, RHAG, RHCG, and combinations thereof, such as particularly preferably RHBG protein.

In particular embodiments, the interaction between the therapeutic or prophylactic agent as described in the present application and the ammonium transporter gene, RNA, preferably mRNA, or protein, preferably alters the activity or the level or both of said target. For example, the therapeutic or prophylactic agent as disclosed herein may be capable of modulating, such as interfering with or increasing, the expression of said at least one ammonium transporter, such as preferably at least one ammonium transporter selected from the group consisting of RHBG, RHAG, RHCG, and combinations thereof, such as particularly preferably RHBG by a cell, the transport of the at least one ammonium transporter protein, such as preferably at least one ammonium transporter protein selected from the group consisting of RHBG, RHAG, RHCG, and combinations thereof, such as particularly preferably RHBG protein to the cell membrane and/or the binding and/or translocation of ammonia/ammonium to or by the at least one ammonium transporter protein, such as preferably at least one ammonium transporter protein selected from the group consisting of RHBG, RHAG, RHCG, and combinations thereof, such as particularly preferably RHBG protein at the cellular membrane. Substrate recognition (and optionally binding) by said at least one ammonium transporter, such as preferably at least one ammonium transporter selected from the group consisting of RHBG, RHAG, RHCG, and combinations thereof, such as particularly preferably RHBG may be distinguishable (e.g., separately or independently detectable) from substrate translocation, or substrate recognition (and optionally binding) and translocation may constitute a dynamic process, in which substrate recognition (and optionally binding) by said at least one ammonium transporter is not distinguishable (e.g., not separately or independently detectable) from substrate translocation. The impact of an agent on ammonia/ammonium transport by said at least one ammonium transporter, such as preferably at least one ammonium transporter selected from the group consisting of RHBG, RHAG, RHCG, and combinations thereof, such as particularly preferably RHBG can be evaluated as explained elsewhere in this specification, such as for example using stopped-flow spectrofluorimetry and the 2′,7′-bis-(2-carboxyethyl)-5(6)-carboxyfluorescein (BCECF) pH-sensitive probe to follow alterations in intracellular pH (pHᵢ) related to ammonium transport, substantially as described by Zidi-Yahiaoui et al. By means of an example and without limitation, the application of this technique to measure the impact of mercury and copper on RHCG-mediated alkalinisation of liposomes is described by Mouro-Chanteloup et al. PLoS One. 2010, 5(1): e8921, which can be readily modified and adapted by the skilled person for measurement of ammonia/ammonium transport by any ammonium transporter, such as RHBG or RHAG.

In other embodiments, the therapeutic or prophylactic agent is capable of interacting with a naturally-occurring binding and/or regulatory partner of said at least one ammonium transporter, such as preferably at least one ammonium transporter selected from the group consisting of RHBG, RHAG, RHCG, and combinations thereof, such as particularly preferably RHBG, or with a gene or RNA, preferably mRNA, encoding said partner.

In particular embodiments, the therapeutic or prophylactic agent for use according to the invention is selected from the group consisting of a protein, a polypeptide, a peptide, a peptidomimetic, a nucleic acid, an aptamer, a small organic molecule, and a compound as defined elsewhere in the specification. or combination of any two or more thereof; preferably wherein said agent is a gene-editing system, an antisense agent, an RNAi agent, such as siRNA or shRNA, or an antibody or functional fragment thereof, or a soluble receptor.

The term “soluble receptor” generally refers to the soluble (i.e., circulating, not bound to a cell) form of a cell-surface molecule, e.g., a cell-surface receptor, or a fragment or derivative thereof. For example, a cell-surface molecule can be made soluble by attaching a soluble fusion partner, e.g., an immunoglobulin (Ig) moiety, or a portion thereof, to the extracellular domain, or by removing its transmembrane domain.

Targeted genome modification is a powerful tool for genetic manipulation of cells and organisms, including mammals. Genome modification or gene editing, including insertion, deletion or replacement of DNA in the genome, can be carried out using a variety of known gene editing systems. The term “gene editing system” or “genome editing system” as used herein refers to a tool to induce one or more nucleic acid modifications, such as DNA or RNA modifications, into a specific DNA or RNA sequence within a cell. Gene editing systems typically make use of an agent capable of inducing a nucleic acid modification. In certain embodiments, the agent capable of inducing a nucleic acid modification may be a (endo)nuclease or a variant thereof having altered or modified activity. (endo)Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through nonhomologous end-joining or homologous recombination, resulting in targeted mutations. In certain embodiments, said (endo)nuclease may be RNA-guided. In certain embodiments, said (endo)nuclease can be engineered nuclease such as a Clustered Regularly Interspaced Short Palindromic Repeats (CRISPR) associated (Cas) (endo)nuclease, such as Cas9, Cpf1, or C2c2, a (zinc finger nuclease (ZFN), a transcription factor-like effector nuclease (TALEN), a meganuclease, or modifications thereof. Methods for using TALEN technology, Zinc Finger technology and CRISPR/Cas technology are known by the skilled person.

Kits comprising the means for modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG by use of a gene editing system are also provided. Examples of such kits, which may be suitable for modulating, such as inhibiting or increasing, RHBG expression or activity, more particularly of human RHBG, include without limitation those available from the following vendors (“#” stands for catalogue number): OriGene Technologies (Rockville, Md., US) (# KN217600).

In particular embodiments, the therapeutic agent or prophylactic agent as disclosed herein is an antisense agents capable of binding to (annealing with) a sequence region in pre-mRNA or mRNA sequence of said at least one ammonium transporter, such as preferably at least one ammonium transporter selected from the group consisting of RHBG, RHAG, RHCG, and combinations thereof, such as particularly preferably RHBG. Particularly intended may be such RNAi agents configured to target mRNA of said at least one ammonium transporter, such as preferably at least one ammonium transporter selected from the group consisting of RHBG, RHAG, RHCG, and combinations thereof, such as particularly preferably RHBG.

The term “antisense” generally refers to an agent (e.g., an oligonucleotide as defined elsewhere in the specification) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein may typically be capable of annealing with (hybridising to) the respective target nucleic acid sequences at high stringency conditions, and capable of hybridising specifically to the target under physiological conditions.

The terms “complementary” or “complementarity” as used herein with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The sequence of an antisense agent need not be 100% complementary to that of its target sequence to bind or hybridise specifically with the latter as defined elsewhere in the specification. An antisense agent may be said to be specifically hybridisable when binding of the agent to a target nucleic acid molecule interferes with the normal function of the target nucleic acid such as to attain an intended outcome (e.g., loss of utility), and there is a sufficient degree of complementarity to avoid non-specific binding of the antisense agent to non-target sequences under conditions in which specific binding is desired, i.e., under physiological conditions in the case of in vivo assays or therapeutic treatment, and in the case of in vitro assays, under conditions in which the assays are performed. Thus, “specifically hybridisable” and “complementary” may indicate a sufficient degree of complementarity or precise pairing such that stable and specific binding occurs between an antisense agent and a nucleic acid target.

Preferably, to ensure specificity of antisense agents towards the desired target over unrelated molecules, the sequence of said antisense agents may be at least about 80% identical, preferably at least about 90% identical, more preferably at least about 95% identical, such as, e.g., about 96%, about 97%, about 98%, about 99% and up to 100% identical to the respective target sequence.

Antisense agents as intended herein preferably comprise or denote antisense molecules such as more preferably antisense nucleic acid molecules or antisense nucleic acid analogue molecules. Preferably, antisense agents may refer to antisense oligonucleotides or antisense oligonucleotide analogues.

Antisense agents such as oligonucleotides as taught herein may be further conjugated (e.g., covalently or non-covalently, directly or via a suitable linker) to one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given agent to be uniformly modified, and in fact more than one of the aforementioned modifications may be incorporated in a single agent or even at a single nucleoside within an oligonucleotide. Further included are antisense compounds that are chimeric compounds. “Chimeric” antisense compounds or “chimeras” are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

The term “RNA interference agent” or “RNAi agent” refers to ribonucleic acid sequences, modified ribonucleic acid sequences, or DNA sequences encoding said ribonucleic acid sequences, which cause RNA interference and thus decrease expression of the target gene.

An RNAi (RNA interference) agent typically comprises, consists essentially of or consists of a double-stranded portion or region (notwithstanding the optional and potentially preferred presence of single-stranded overhangs) of annealed complementary strands, one of which has a sequence corresponding to a target nucleotide sequence (hence, to at least a portion of an mRNA) of the target gene to be down-regulated. The other strand of the RNAi agent is complementary to said target nucleotide sequence. Non-limiting examples of RNAi agents are shRNAs, siRNAs, miRNAs, and DNA-RNA hybrids.

Whereas the sequence of an RNAi agent need not be completely identical to a target sequence to be down-regulated, the number of mismatches between a target sequence and a nucleotide sequence of the RNAi agent is preferably no more than 1 in 5 bases, or 1 in 10 bases, or 1 in 20 bases, or 1 in 50 bases.

Preferably, to ensure specificity of RNAi agents towards the desired target over unrelated molecules, the sequence of said RNAi agents may be at least about 80% identical, preferably at least about 90% identical, more preferably at least about 95% identical, such as, e.g., about 96%, about 97%, about 98%, about 99% and up to 100% identical to the respective target sequence.

In particular embodiments, the therapeutic agent for use according to present invention is an RNAi agent of between about 15 and about 60 nucleotides in length and which comprises a nucleotide sequence that is at least 70% identical to a region of the gene encoding said at least one ammonium transporter, such as preferably at least one ammonium transporter selected from the group consisting of RHBG, RHAG, RHCG, and combinations thereof, such as particularly preferably.

An RNAi agent may be formed by separate sense and antisense strands or, alternatively, by a common strand providing for fold-back stem-loop or hairpin design where the two annealed strands of an RNAi agent are covalently linked.

An siRNA molecule may be typically produced, e.g., synthesised, as a double stranded molecule of separate, substantially complementary strands, wherein each strand is about 18 to about 35 bases long, preferably about 19 to about 30 bases, more preferably about 20 to about 25 bases and even more preferably about 21 to about 23 bases. shRNA is in the form of a hairpin structure. shRNA can be synthesized exogenously or can be formed by transcribing from RNA polymerase III promoters in vivo. Preferably, shRNAs can be engineered in host cells or organisms to ensure continuous and stable suppression of a desired gene. It is known that siRNA can be produced by processing a hairpin RNA in cells.

RNAi agents as intended herein may include any modifications as set out herein for nucleic acids and oligonucleotides, in order to improve their therapeutic properties.

In embodiments, at least one strand of an RNAi molecules may have a 3′ overhang from about 1 to about 6 bases in length, e.g., from 2 to 4 bases, more preferably from 1 to 3 bases. For example, one strand may have a 3′ overhang and the other strand may be either blunt-ended or may also have a 3′overhang. The length of the overhangs may be the same or different for each strand. The 3′ overhangs can be stabilised against degradation. For example, the RNA may be stabilised by including purine nucleotides, such as A or G nucleotides. Alternatively, substitution of pyrimidine nucleotides by modified analogues, e.g., substitution of U 3′ overhangs by 2′-deoxythymidine is tolerated and does not affect the efficiency of RNAi.

An exemplary but non-limiting siRNA molecule may by characterized by any one or more, and preferably by all of the following criteria:
at least about 80% sequence identity, more preferably at least about 90% or at least about 95% or at least about 97% sequence identity to target mRNA;
having a sequence which targets an area of the target gene present in mature mRNA (e.g., an exon or alternatively spliced intron);
showing a preference for targeting the 3′ end of the target gene.

The exemplary siRNA may be further characterised by one or more or all of the following criteria:
having a double-stranded nucleic acid length of between 16 to 30 bases and preferably of between 18 to 23 bases, and preferably of 19 nucleotides;
having GC content between about 30 and about 50%
having a TT(T) sequence at 3′ end;
showing no secondary structure when adopting the duplex form;
having a Tm (melting temperature) of lower than 20° C.
having the nucleotides indicated here below in the sequence of the nucleotides, wherein “h” is A, C, T/U but not G; wherein “d” is A, G, T/U but not C, and wherein “w” is A or T/U, but not G or C:

| | | | | | | | | | | | | | | | | | | | | | | |
|---|---|---|---|---|---|---|---|---|---|---|---|---|---|---|---|---|---|---|---|---|---|---|
| — | — | 1 | 2 | 3 | 4 | 5 | 6 | 7 | 8 | 9 | 10 | 11 | 12 | 13 | 14 | 15 | 16 | 17 | 18 | 19 | — | — |

| | | | | | | | | | | |
|---|---|---|---|---|---|---|---|---|---|---|
| mRNA | P′5 | A | A | A | U | h | w | | | 3′-OH |
| si- | OH-3′ | T | T | U | A | d | w | | | 5′-P |
| ASense | | | | | | | | | | |
| si- | P-5′ | | | A | U | h | w | T | T | 3′-OH |
| Sense | | | | | | | | | | |

RNAi agents as intended herein may particularly comprise or denote (i.e., may be selected from a group comprising or consisting of) RNAi nucleic acid molecules or RNAi nucleic acid analogue molecules, such as preferably short interfering nucleic acids and short interfering nucleic acid analogues (siNA) such as short interfering RNA and short interfering RNA analogues (siRNA), and may further denote inter alia double-stranded RNA and double-stranded RNA analogues (dsRNA), micro-RNA and micro-RNA analogues (miRNA), and short hairpin RNA and short hairpin RNA analogues (shRNA).

Production of antisense agents and RNAi agents can be carried out by any processes known in the art, such as inter alia partly or entirely by chemical synthesis (e.g., routinely known solid phase synthesis; an exemplary an non-limiting method for synthesising oligonucleotides on a modified solid support is described in U.S. Pat. No. 4,458,066; in another example, diethyl-phosphoramidites are used as starting materials and may be synthesised as described by Beaucage et al. 1981 (Tetrahedron Letters 22: 1859-1862)), or partly or entirely by biochemical (enzymatic) synthesis, e.g., by in vitro transcription from a nucleic acid construct (template) using a suitable polymerase such as a T7 or SP6 RNA polymerase, or by recombinant nucleic acid techniques, e.g., expression from a vector in a host cell or host organism. Nucleotide analogues can be introduced by in vitro chemical or biochemical synthesis. In an embodiment, the antisense agents of the invention are synthesised in vitro and do not include antisense compositions of biological origin, or genetic vector constructs designed to direct the in vivo synthesis of antisense molecules.

In particular embodiments, the therapeutic or prophylactic agent for use according to the invention may be an siRNA directed against one of the following target sequences: UGGUCUUCCUGCAGCGUUA (SEQ ID NO: 16), AUUCAGACCUCUUCGCCAU (SEQ ID NO: 17), CCACAGUAACGCGGACAAU (SEQ ID NO: 18), or GGCCGUAACUGGGUACAAU (SEQ ID NO: 19), or a combination of siRNAs directed against any two, any three, or all four of said target sequences.

In particular embodiments, a small organic molecule suitable as a therapeutic agent herein may include 4,4′-diisothiocyano-2,2′-disulfonic stilbene (DIDS), a stilbene derivative, which is able to block both gas (NH₃) and ionic (NH₄⁺) transport by said at least one ammonium transporter, such as preferably at least one ammonium transporter selected from the group consisting of RHBG, RHAG, RHCG, and combinations thereof, such as particularly preferably RHBG.

In particular embodiments, the therapeutic or prophylactic agent according to the invention is a polyclonal antibody, a monoclonal antibody, a chimeric antibody, a humanized antibody, a primatized antibody, a human antibody, a Nanobody or mixtures thereof, as defined elsewhere in the specification. Non-limiting examples of monoclonal antibodies potentially suitable for use as therapeutic agents herein include without limitation the antibody commercially available from Santa Cruz (cat. no. # sc-398816) which is a mouse monoclonal antibody (clone B-9, IgG1 (kappa light chain)) directed against residues 337-408, which are predicted to reside in the two last transmembrane domains of RHBG and the extracellular loop in between; or fragments thereof, human/mouse chimeric antibodies thereof, or humanized antibodies thereof.

In particular embodiments, the therapeutic or prophylactic agent as disclosed herein may be an intrabody, as defined elsewhere in the specification, specifically directed to the at least one ammonium transporter protein, such as preferably at least one ammonium transporter protein selected from the group consisting of RHBG, RHAG, RHCG, and combinations thereof, such as particularly preferably RHBG protein, which may bind said at least one ammonium transporter protein intracellularly and thereby modulate, such as inhibit or increase, activity of said at least one ammonium transporter, for instance, by preventing said at least one ammonium transporter from reaching the cell surface.

The therapeutic or prophylactic agent as disclosed herein may be an expressible molecule such as an antibody or a fragment or derivative thereof, a protein or polypeptide, a peptide, a nucleic acid, an antisense agent or an RNAi agent, it shall be understood that the agent itself may be introduced to a subject or may be introduced by means of a recombinant nucleic acid comprising a sequence encoding the agonist operably linked to one or more regulatory sequences allowing for expression of said sequence encoding the agent (e.g., gene therapy or cell therapy).

Hence, an agent may comprise a recombinant nucleic acid comprising a sequence encoding one or more desired proteins, polypeptides, peptides, antisense agents or RNAi agents, operably linked to one or more regulatory sequences allowing for expression of said sequence or sequences encoding the proteins, polypeptides, peptides, antisense agents or RNAi agents, e.g., in vitro, in a host cell, host organ and/or host organism (expression constructs). Such recombinant nucleic acid may be comprised in a suitable vector.

By “encoding” is meant that a nucleic acid sequence or part(s) thereof corresponds, by virtue of the genetic code of an organism in question to a particular amino acid sequence, e.g., the amino acid sequence of one or more desired proteins or polypeptides, or to another nucleic acid sequence in a template-transcription product (e.g. RNA or RNA analogue) relationship.

Preferably, a nucleic acid encoding one or more proteins, polypeptides or peptides may comprise one or more open reading frames (ORF) encoding said one or more proteins, polypeptides or peptides. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a protein, polypeptide or peptide. Hence, the term may be synonymous with “coding sequence” as used in the art.

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence.

The precise nature of regulatory sequences or elements required for expression may vary between expression environments, but typically include a promoter and a transcription terminator, and optionally an enhancer.

Reference to a “promoter” or “enhancer” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgarno sequence).

In embodiments, promoters contemplated herein may be constitutive or inducible.

The terms “terminator” or “transcription terminator” refer generally to a sequence element at the end of a transcriptional unit which signals termination of transcription. For example, a terminator is usually positioned downstream of, i.e., 3′ of ORF(s) encoding a polypeptide of interest. For instance, where a recombinant nucleic acid contains two or more ORFs, e.g., successively ordered and forming together a multi-cistronic transcription unit, a transcription terminator may be advantageously positioned 3′ to the most downstream ORF.

The term “vector” generally refers to a nucleic acid molecule, typically DNA, to which nucleic acid segments may be inserted and cloned, i.e., propagated. Hence, a vector will typically contain one or more unique restriction sites, and may be capable of autonomous replication in a defined host or vehicle organism such that the cloned sequence is reproducible. Vectors may include, without limitation, plasmids, phagemids, bacteriophages, bacteriophage-derived vectors, PAC, BAC, linear nucleic acids, e.g., linear DNA, viral vectors, etc., as appropriate. Expression vectors are generally configured to allow for and/or effect the expression of nucleic acids or ORFs introduced thereto in a desired expression system, e.g., in vitro, in a host cell, host organ and/or host organism. For example, expression vectors may advantageously comprise suitable regulatory sequences.

As noted elsewhere, an agent may comprise a protein, polypeptide or peptide. Such may be suitably obtained through expression by host cells or host organisms, transformed with an expression construct encoding and configured for expression of said protein, polypeptide or peptide in said host cells or host organisms, followed by purification of the protein, polypeptide or peptide.

The terms “host cell” and “host organism” may suitably refer to cells or organisms encompassing both prokaryotes, such as bacteria, and eukaryotes, such as yeast, fungi, protozoan, plants and animals. Contemplated as host cells are inter alia unicellular organisms, such as bacteria (e.g., E. coli, Salmonella tymphimurium, Serratia marcescens, or Bacillus subtilis), yeast (e.g., Saccharomyces cerevisiae or Pichia pastoris), (cultured) plant cells (e.g., from Arabidopsis thaliana or Nicotiana tobaccum) and (cultured) animal cells (e.g., vertebrate animal cells, mammalian cells, primate cells, human cells or insect cells). Contemplated as host organisms are inter alia multi-cellular organisms, such as plants and animals, preferably animals, more preferably warm-blooded animals, even more preferably vertebrate animals, still more preferably mammals, yet more preferably primates; particularly contemplated are such animals and animal categories which are non-human.

Such protein, polypeptide or peptide may be suitably isolated. The term “isolated” with reference to a particular component (such as for instance a nucleic acid, protein, polypeptide or peptide) generally denotes that such component exists in separation from—for example, has been separated from or prepared and/or maintained in separation from—one or more other components of its natural environment. For instance, an isolated human or animal protein or complex may exist in separation from a human or animal body where it naturally occurs. The term “isolated” as used herein may preferably also encompass the qualifier “purified” as defined elsewhere in the specification

Further, there are several well-known methods of introducing nucleic acids (e.g., antisense and RNAi agents) into animal cells, any of which may be used herein. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors (i.e. derived from lentivirus, adeno-associated virus, adenovirus, retrovirus or antiviruses), electroporation, and the like. Other techniques or methods which are suitable for delivering nucleic acid molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable micropsheres. Also envisaged is encapsulation of NA in various types of liposomes (immunoliposomes, PEGylated (immuno) liposomes), cationic lipids and polymers, nanoparticules or dendrimers, poly (lactic-Co-Glycolic Acid) polymeric microspheres, implantable drug-releasing biodegradable microspheres, etc; and co-injection of NA with protective agent like the nuclease inhibitor aurintricarboxylic acid. It shall be clear that also a combination of different above-mentioned delivery modes or methods may be used.

In particular embodiments, the therapeutic or prophylactic agent is administered in a therapeutically effective amount.

In particular embodiments, the therapeutic or prophylactic agent capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG as taught herein is formulated into and administered as pharmaceutical formulations or compositions. Such pharmaceutical formulations or compositions may be comprised in a kit of parts.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions of the invention may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agents and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The dosage or amount of the present active substances used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage of the agent capable of modulating, such as inhibiting or increasing, expression or activity said at least one ammonium transporter, such as preferably at least one ammonium transporter selected from the group consisting of RHBG, RHAG, RHCG, and combinations thereof, such as particularly preferably RHBG as taught herein might range from about 1 μg/kg to 1 μg/kg of body weight or more, depending on the factors mentioned above. For instance, a daily dosage of the agent capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG as taught herein may range from about 1 mg/kg to 1 μg/kg of body weight. For repeated administrations over several days or longer, depending on the condition, the treatment is sustained until a desired suppression of disease symptoms occurs. A preferred dosage of the agent capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG as taught herein may be in the range from about 10.0 mg/kg to about 500 mg/kg of body weight. Thus, one or more doses of about 10.0 mg/kg, 20.0 mg/kg, 50.0 mg/kg, 100 mg/kg, 200 mg/kg, 300 mg/kg, 400 mg/kg, or 500 mg/kg (or any combination thereof) may be administered to the patient. Such doses may be administered intermittently, e.g., every day, every week or every two or three weeks.

In certain embodiments, the agent capable of modulating, such as inhibiting or increasing, expression or activity of said at least one ammonium transporter, such as preferably at least one ammonium transporter selected from the group consisting of RHBG, RHAG, RHCG, and combinations thereof, such as particularly preferably RHBG may be administered daily during the treatment. In certain embodiments, the agent capable of modulating, such as inhibiting or increasing, expression or activity of said at least one ammonium transporter may be administered at least once a day during the treatment, for example the agent capable of modulating, such as inhibiting or increasing, expression or activity of said at least one ammonium transporter may be administered at least twice a day during the treatment, for example the agent capable of modulating, such as inhibiting or increasing, expression or activity of said at least one ammonium transporter may be administered at least three times a day during the treatment. In certain embodiments, the agent capable of modulating, such as inhibiting or increasing, expression or activity of said at least one ammonium transporter may be administered continuously during the treatment for instance in an aqueous drinking solution.

Aspects and embodiments of the present invention hence encompass, and the present specification describes, subject-matter as set forth in any one and all of the following Statements:

Statement 1. Use of at least one ammonium transporter as a biomarker for a proliferative disease in a subject.

Statement 2. The use according to statement 1, wherein the at least one ammonium transporter belongs to the Rhesus (Rh) protein family.

Statement 3. The use according to statement 1, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 4. Use of RHBG as a biomarker for a proliferative disease in a subject.

Statement 5. The use according to any one of statements 1-4 for the diagnosis, prediction, prognosis and/or monitoring of said proliferative disease in the subject.

Statement 6. A method for the diagnosis, prediction, prognosis and/or monitoring of a proliferative disease in a subject or for determining whether a subject is in need of therapeutic or prophylactic treatment of a proliferative disease, comprising detecting at least one ammonium transporter in a tissue sample from the subject.

Statement 7. The method according to statement 6, wherein the at least one ammonium transporter belongs to the Rh family.

Statement 8. The method according to statement 6 or 7, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 9. A method for the diagnosis, prediction, prognosis and/or monitoring of a proliferative disease in a subject or for determining whether a subject is in need of therapeutic or prophylactic treatment of a proliferative disease, comprising detecting RHBG in a tissue sample from the subject.

Statement 10. The method according to any one of statements 6-9, comprising the step of comparing the quantity or expression level of said at least one ammonium transporter, such as preferably at least one ammonium transporter selected from the group consisting of RHBG, RHAG, RHCG, and combinations thereof, such as particularly preferably RHBG, in said sample with the reference quantity or expression level of said at least one ammonium transporter.

Statement 11. The method according to any one of statements 6-10 comprising:
(i) determining the quantity or expression level of said at least one ammonium transporter, such as preferably at least one ammonium transporter selected from the group consisting of RHBG, RHAG, RHCG, and combinations thereof, such as particularly preferably RHBG, in a tissue sample from the subject;
(ii) comparing the quantity or expression level of said at least one ammonium transporter as determined in (a) with a reference value, said reference value representing a known diagnosis, prediction and/or prognosis of said proliferative disease;
(iii) finding a deviation or no deviation of the quantity or expression level of said at least one ammonium transporter as determined in (a) from said reference value;
(iv) attributing said finding of deviation or no deviation to a particular diagnosis, prediction, or prognosis of the proliferative disease in the subject.

Statement 12. The method according to statement 11, wherein in step (iii) an elevated quantity or expression level of said at least one ammonium transporter, such as preferably at least one ammonium transporter selected from the group consisting of RHBG, RHAG, RHCG, and combinations thereof, such as particularly preferably RHBG, in the tissue sample from the subject as compared to the reference value allows for the diagnosis, prediction, or prognosis of the proliferative disease in the subject.

Statement 13. The method according to statement 11 or 12, wherein said reference value is determined from a tissue not affected by the proliferative disease, such as wherein said reference value is determined from healthy tissue.

Statement 14. A therapeutic or prophylactic agent for use as a medicament, preferably for use as a medicament in the treatment of a proliferative disease, wherein said therapeutic agent is capable of modulating, such as inhibiting or increasing, expression or activity of at least one ammonium transporter.

Statement 15. The therapeutic or prophylactic agent for use according to statement 14, wherein the at least one ammonium transporter belongs to the Rh family.

Statement 16. The therapeutic or prophylactic agent for use according to statement 14 or 15, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 17. A therapeutic or prophylactic agent for use as a medicament, preferably for use as a medicament in the treatment of a proliferative disease, wherein said therapeutic agent is capable of modulating, such as inhibiting or increasing, RHBG expression or activity.

Statement 18. The therapeutic or prophylactic agent according to any one of statements 14-17, wherein the agent modulates, such as interferes with or increases, ammonia transport across the cell membrane mediated by said at least one ammonium transporter.

Statement 19. The therapeutic or prophylactic agent according to any one of statements 14-18, wherein said agent is capable of interacting with the gene or RNA, preferably mRNA, encoding said at least one ammonium transporter, or the agent is capable of interacting with said at least one ammonium transporter protein, or wherein said agent is capable of interacting with a naturally-occurring binding and/or regulatory partner of said at least one ammonium transporter, or with a gene or RNA, preferably mRNA, encoding said partner.

Statement 20. The therapeutic or prophylactic agent for use according to any one of statements 14-19, wherein said therapeutic or prophylactic agent is selected from the group consisting of a protein, a polypeptide, a peptide, a peptidomimetic, a nucleic acid, an aptamer, a small organic molecule, and a compound or combination of any two or more thereof; preferably wherein said agent is a gene-editing system, an antisense agent, an RNAi agent, such as siRNA or shRNA, or an antibody or functional fragment thereof, or a soluble receptor.

Statement 21. The use, method, or agent for use according to any of the preceding statements, wherein said proliferative disease is selected form the group consisting of hepatocellular carcinoma, hepatoma, hepatic carcinoma, liver cancer, colorectal cancer, colon cancer or carcinoma, and rectal cancer.

Statement 22. The use, method, or agent for use according to any one of statements 1 to 20, wherein said proliferative disease is selected form the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, endometrial cancer, uterine carcinoma, salivary gland carcinoma, vulval cancer, thyroid cancer, anal carcinoma, penile carcinoma, head and neck cancer, adrenal gland cancer, paraganglioma, oesophageal cancer or carcinoma, kidney or renal cancer or carcinoma, urothelial cancer, bile duct cancer, prostate cancer, brain cancer, leukemia, and multiple myeloma.

Statement 23. The use, method, or agent for use according to any of the preceding statements, wherein said proliferative disease is characterised by (over)expression of said at least one ammonium transporter, such as preferably at least one ammonium transporter selected from the group consisting of RHBG, RHAG, RHCG, and combinations thereof, such as particularly preferably RHBG.

Statement 24. The use, method, or agent for use according to any of the preceding statements, wherein said proliferative disease is luminal breast cancer.

Statement 25. An antibody or a functional fragment thereof, characterised in that the antibody or functional fragment thereof binds epitope DSPPRLPALRGPSS of human RHBG as set out in SEQ ID NO: 15.

Statement 26. A kit for diagnosing, predicting, prognosing and/or monitoring a proliferative disease in a subject, the kit comprising:
(i) means for measuring the quantity or expression level of at least one ammonium transporter in a tissue sample from a subject; and
(ii) a reference value of the quantity or expression level of said at least one ammonium transporter or means for establishing said reference value, wherein said reference value represents a known diagnosis, prediction and/or prognosis of the proliferative disease, such as wherein said reference value corresponds to the quantity or expression level of said at least one ammonium transporter in a tissue not affected by the proliferative disease, such as in a healthy tissue, or wherein said reference value corresponds to the quantity or expression level of said at least one ammonium transporter in a tissue affected by the proliferative disease.

Statement 27. A kit for diagnosing, predicting, prognosing and/or monitoring luminal breast cancer in a subject, the kit comprising:
(i) means for measuring the quantity or expression level of at least one ammonium transporter in a tissue sample obtained from a breast tumour from a subject; and
(ii) a reference value of the quantity of said at least one ammonium transporter or means for establishing said reference value, wherein said reference value represents the quantity or expression level of said at least one ammonium transporter in a healthy breast tissue or in a basal breast cancer tissue.

Statement 28. The kit according to statement 26 or 27, wherein the at least one ammonium transporter belongs to the Rh family.

Statement 29. The kit according to statement 26 or 27, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 30. A kit for diagnosing, predicting, prognosing and/or monitoring a proliferative disease in a subject, the kit comprising:
(i) means for measuring the quantity or expression level of RHBG in a tissue sample from a subject; and
(ii) a reference value of the quantity or expression level of RHBG or means for establishing said reference value, wherein said reference value represents a known diagnosis, prediction and/or prognosis of the proliferative disease, such as wherein said reference value corresponds to the quantity or expression level of RHBG in a tissue not affected by the proliferative disease, such as in a healthy tissue, or wherein said reference value corresponds to the quantity or expression level of RHBG in a tissue affected by the proliferative disease.

Statement 31. A kit for diagnosing, predicting, prognosing and/or monitoring luminal breast cancer in a subject, the kit comprising:
(i) means for measuring the quantity or expression level of RHBG in a tissue sample obtained from a breast tumour from a subject; and
(ii) a reference value of the quantity of RHBG or means for establishing said reference value, wherein said reference value represents the quantity or expression level of RHBG in a healthy breast tissue or in a basal breast cancer tissue.

Statement 32. Use of the kit according to any one of statements 26-31 for the diagnosis, prediction, prognosis and/or monitoring of the proliferative disease in a subject.

Statement 33. Use of the kit according to any one of statements 26-31 for the diagnosis, prediction, prognosis and/or monitoring of luminal breast cancer in a subject.

Statement 34. The therapeutic or prophylactic agent for use according to statement 20, wherein said antibody is a polyclonal antibody, a monoclonal antibody, a chimeric antibody, a humanized antibody, a primatized antibody, a human antibody, a Nanobody or mixtures thereof.

Statement 35. The therapeutic agent for use according to statement 19, wherein said RNAi agent is between about 15 and about 60 nucleotides in length and comprises a nucleotide sequence that is at least 70% identical to a region of the gene encoding said at least one ammonium transporter, such as preferably at least one ammonium transporter selected from the group consisting of RHBG, RHAG, RHCG, and combinations thereof, such as particularly preferably RHBG.

Statement 36. A method for diagnosing and treating a proliferative disease in a subject comprising:
(i) detecting at least one ammonium transporter in a tissue sample from the subject;
(ii) diagnosing the subject as in need of treatment of the proliferative disease when said at least one ammonium transporter is detected in the tissue sample; and
(iii) administering an effective amount of a treatment to the diagnosed subject.

Statement 37. A method for diagnosing and treating a proliferative disease in a subject comprising:
(i) determining the quantity or expression level of at least one ammonium transporter in a tissue sample from the subject;
(ii) comparing the quantity or expression level of said at least one ammonium transporter as determined in (i) with a reference value, said reference value representing a known diagnosis of said proliferative disease;
(iii) diagnosing the subject as in need of treatment of the proliferative disease when said quantity or expression level of said at least one ammonium transporter as determined in (i) deviates from said reference value, and
(iv) administering an effective amount of a treatment to the diagnosed subject.

Statement 38. The method according to statement 36 or 37, wherein the at least one ammonium transporter belongs to the Rh family.

Statement 39. The method according to any one of statement 36-38, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 40. The method according to any one of statements 37-39, wherein in step (iii) an elevated quantity or expression level of said at least one ammonium transporter, such as preferably at least one ammonium transporter selected from the group consisting of RHBG, RHAG, RHCG, and combinations thereof, such as particularly preferably RHBG, in a tissue sample obtained from the tissue sample from the subject as compared to the reference value allows for the diagnosis of the proliferative disease in the subject.

Statement 41. The method according to any one of statements 37-40, wherein said reference value is determined from a tissue not affected by the proliferative disease, such as wherein said reference value is determined from healthy tissue.

Statement 42. A method for diagnosing and treating a proliferative disease in a subject comprising:
(i) detecting RHBG in a tissue sample from the subject;
(ii) diagnosing the subject as in need of treatment of the proliferative disease when RHBG is detected in the tissue sample; and
(iv) administering an effective amount of a treatment to the diagnosed subject.

Statement 43. A method for diagnosing and treating a proliferative disease in a subject comprising:
(i) determining the quantity or expression level of RHBG in a tissue sample from the subject;
(ii) comparing the quantity or expression level of RHBG as determined in (i) with a reference value, said reference value representing a known diagnosis of said proliferative disease;
(iii) diagnosing the subject as in need of treatment of the proliferative disease when said quantity or expression level of RHBG as determined in (i) deviates from said reference value, and
(iv) administering an effective amount of a treatment to the diagnosed subject.

Statement 44. A method for diagnosing and treating luminal breast cancer in a subject comprising:
(i) determining the quantity or expression level of at least one ammonium transporter in a tissue sample obtained from a breast tumour from the subject;
(ii) comparing the quantity or expression level of said at least one ammonium transporter as determined in (i) with a reference value, said reference value representing the quantity or expression level of said at least one ammonium transporter in a healthy breast tissue or in a basal breast cancer tissue;
(iii) diagnosing the subject as in need of treatment of luminal breast cancer when said quantity or expression level of said at least one ammonium transporter as determined in (i) deviates from said reference value, and
(iv) administering an effective amount of a treatment to the diagnosed subject.

Statement 45. The method according to statement 44, wherein the at least one ammonium transporter belongs to the Rh family.

Statement 46. The method according to statement 44 or 45, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 47. The method according to any one of statements 44-46, wherein in step (iii) an elevated quantity or expression level of said at least one ammonium transporter in a tissue sample obtained from the tissue sample from the subject as compared to the reference value allows for the diagnosis of luminal breast cancer in the subject.

Statement 48. A method for diagnosing and treating luminal breast cancer in a subject comprising:
(i) determining the quantity or expression level of RHBG in a tissue sample obtained from a breast tumour from the subject;
(ii) comparing the quantity or expression level of RHBG as determined in (i) with a reference value, said reference value representing the quantity or expression level of RHBG in a healthy breast tissue or in a basal breast cancer tissue;
(iii) diagnosing the subject as in need of treatment of luminal breast cancer when said quantity or expression level of RHBG as determined in (i) deviates from said reference value, and
(iv) administering an effective amount of a treatment to the diagnosed subject.

Statement 49. The methods according to any one of statements 36-48, wherein the treatment is selected from the group consisting of radiotherapy, chemotherapy, hormone therapy, biological therapy, bisphosphonate therapy, immune therapy, stem cell therapy, and surgery.

Statement 50. The method according to statement 49, wherein the treatment is biological therapy, preferably therapy using the agent according to any one of statements 14-20.

Statement 51. A method for detecting or measuring the quantity or expression level of at least one ammonium transporter in a subject, said method comprising:
(i) obtaining a tissue sample from the subject; and
(ii) detecting or measuring the quantity or expression level of said at least one ammonium transporter in the tissue sample;
wherein said patient is affected by a proliferative disease.

Statement 52. The method according to statement 51, wherein the at least one ammonium transporter belongs to the Rh family.

Statement 53. The method according to statement 51 or 52, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 54. A method for detecting or measuring the quantity or expression level of RHBG in a subject, said method comprising:
(i) obtaining a tissue sample from the subject; and
(ii) detecting or measuring the quantity or expression level of RHBG in the tissue sample;

wherein said patient is affected by a proliferative disease.

Statement 55. The method according to any one of statements 51-54, wherein the proliferative disease is breast cancer.

Statement 56. The method according to any one of statements 51-55, wherein detecting or measuring the quantity or expression level of RHBG in the tissue sample comprises contacting the tissue sample with the antibody as defined in statement XIV and detecting binding between RHBG and the antibody.

Statement 57. A method of treatment of a proliferative disease in a subject in need thereof, comprising administering to the subject a therapeutically or prophylactically effective amount of a therapeutic or prophylactic agent capable of modulating, such as inhibiting or increasing, expression or activity of at least one ammonium transporter.

Statement 58. The method according to statement 57, wherein the at least one ammonium transporter belongs to the Rh family.

Statement 59. The method according to statement 57 or 58, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

Statement 60. An immunoassay for RHBG comprising the antibody as defined in statement 23. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Example 1: RHBG is Overexpressed in Luminal Breast Cancer Cell Lines

Expression of human RHBG was determined by qRT-PCR in three Triple negative (or basal) breast cancer cell lines, namely (i) HCC38 (ER−) (ATCC-CRL-2314), which is a cell line obtained from human breast ductal carcinoma, (ii) Sum149PT (ER−), which is a cell line obtained from human invasive ductal carcinoma of the breast and (iii) Sum159PT (ER−), which is a cell line obtained from human anaplastic carcinoma of the breast; and three luminal breast cancer cell lines, namely (i) MCF-7 (ER+) (ATCC-HTB-22), which is a cell line obtained from human breast adenocarcinoma, (ii) BT-474 (ER+) (ATCC-HTB-20), which is a cell line obtained from human breast ductal carcinoma, and (iii) MDA-MB-361(ER+) (ATCC-HTB-27), which is a cell line obtained from human breast adenocarcinoma; human. These expression levels were compared to the expression levels of human RHBG in primary Human Mammary Epithelial Cells (HMEC; ATCC-PCS-600-010; control). The expression levels of ACTB (housekeeping gene) were used for normalisation of the data.

Table 1 provides the primers and probes used to detect RHBG and the house keeping gene ACTB in Example 1.

TABLE 1
Primer and probe sequences for
measuring expression levels of
RHBG and ACTB

| Gene | | sequence |
|---|---|---|
| RHBG | Forward | CTGTGAGTGCAGCATTGAAG |
| | primer | (SEQ ID NO: 3) |
| | Reverse | CTACCATTCAGACCTCTTCGC |
| | primer | (SEQ ID NO: 4) |
| | Probe | /56-FAM/CCATCTTCC/ |
| | | ZEN/TGTGGATCTTCTGGCC/ |
| | | 3IABkFQ/ |
| | | (SEQ ID NO: 5) |
| ACTB | Forward | AAGTCAGTGTACAGGTAAGCC |
| | primer | (SEQ ID NO: 6) |
| | Reverse | GTCCCCCAACTTGAGATGTATG |
| | primer | (SEQ ID NO: 7) |
| | Probe | /56-FAM/CTGCCTCCA/ZEN/ |
| | | CCCACTCCCA/3IABkFQ/ |
| | | (SEQ ID NO: 8) |

“56-FAM” refers to 5′ 6-FAM (6-carboxyfluorescein); “ZEN” refers to the ZEN™ internal quencher (Integrated DNA Technologies); and “31ABkFQ” refers to 3′ Iowa Black® FQ quencher (Integrated DNA Technologies).

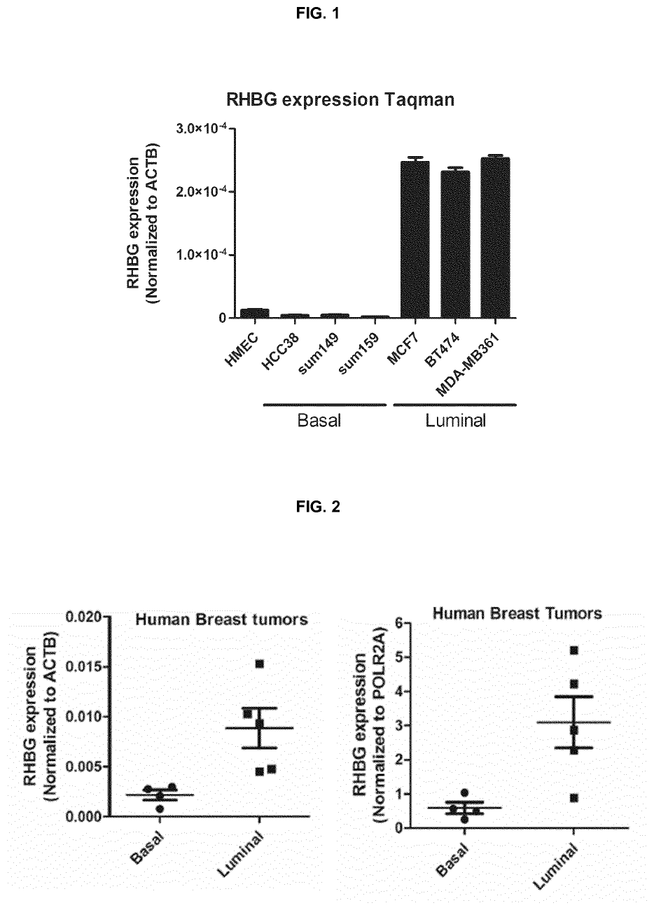

FIG. 1 shows that the expression levels of RHBG were increased in luminal breast cancer cell lines compared to the expression levels of RHBG in basal breast cancer cell lines and also compared to the expression levels of RHBG in control cells (P value<0.0001, the data are presented as mean+/−SEM (n=3); the test that was used was a t-test).

### Example 2: RHBG is Overexpressed in Luminal Breast Tumours (qRT-PCR)

Tissue samples were collected from patients with a basal breast tumour (n=4) or a luminal breast tumours (n=5) by tumour biopsy. Subsequently, these samples were fixated with formalin and embedded in paraffin. Next, RNA was purified from these formalin-fixed, paraffin-embedded specimens and the expression levels of human RHBG were determined by qRT-PCR. The expression levels of ACTB or POLR2A (housekeeping genes) were used to normalise the data.

FIG. 2 shows that the expression levels of RHBG were increased in tissue samples from luminal breast tumours compared to the expression levels of RHBG in in tissue samples from basal breast tumours (P value=0.0232, the test that was used was a t-test).

Table 2 provides the primers used to detect RHBG and the house keeping genes ACTB and POLR2A in Example 2.

TABLE 2
Primer sequences for measuring
expression levels of RHBG,
ACTB and POLR2A

| gene | | sequence |
|---|---|---|
| RHBG | Forward primer | CCTCAAGTGAAATGATGCTG |
| | | (SEQ ID NO: 9) |
| | Reverse primer | ATTTTGATTCAAGGATGGGC |
| | | (SEQ ID NO: 10) |
| ACTB | Forward primer | CTGGAACGGTGAAGGTGACA |
| | | (SEQ ID NO: 11) |
| | Reverse primer | AAGGGACTTCCTGTAACAAT |
| | | GCA (SEQ ID NO: 12) |
| POLR2A | Forward primer | CATCATCATCCATCTTGTCC |
| | | (SEQ ID NO: 13) |
| | Reverse primer | AAGATCAATGCTGGTTTTGG |
| | | (SEQ ID NO: 14) |

### Example 3: RHBG is Overexpressed in Luminal Breast Tumours (Immunohistochemistry)

The RHBG polyclonal antibody (Genecust, Luxembourg) was produced by immunizing New Zealand rabbits with a peptide of the target epitope (DSPPRLPALRGPSS, SEQ ID NO: 15) and subsequently purifying the polyclonal antibody by affinity purification methods. The peptide was conjugated with a carrier protein (keyhole limpet hemocyanin, KLH) via an additional cysteine added at the N-terminus of the peptide, hence, the immunogen was KLH-C-DSPPRLPALRGPSS.

Immunohistochemistry (IHC) for human RHBG protein expression was performed on the Formalin-fixed paraffin-embedded (FFPE) basal (n=4) and luminal (n=5) breast tumours of Example 2 using the RHBG polyclonal antibody.

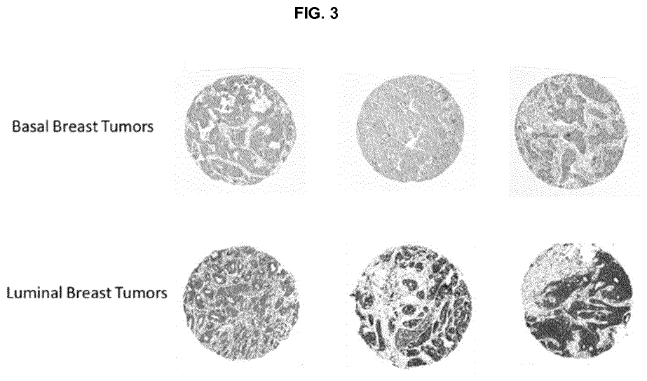

FIG. 3 shows that the IHC-staining for RHBG was stronger in tissue samples of luminal breast tumours when compared to IHC-staining for RHBG in tissue samples of basal breast tumours, which was nearly absent. Accordingly, these data indicate that the protein levels of RHBG are higher in tissue samples of luminal breast tumours when compared to the protein levels of RHBG in tissue samples of basal breast tumours.

### Example 4: Targeted Inhibition of Expression of RHBG with siRNA in Luminal Breast Cancer Cell Lines Inhibits Proliferation

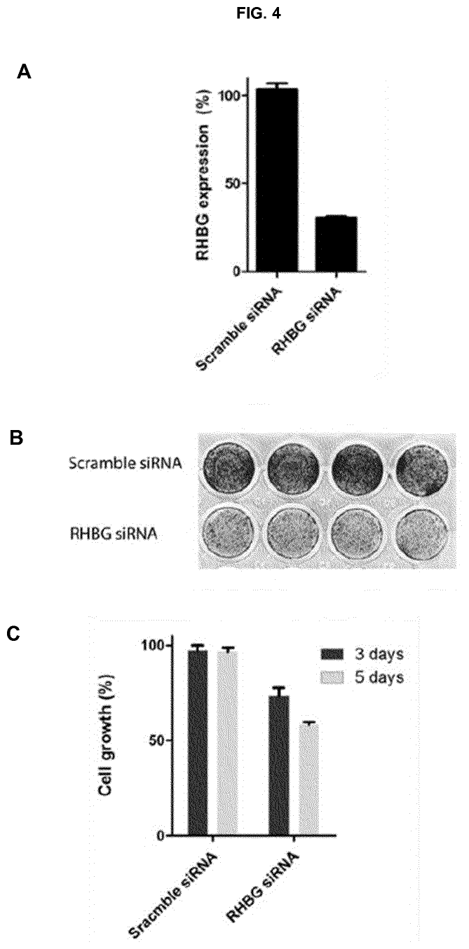

Expression of human RHBG was inhibited with siRNA directed against human RHBG (Table 3; on-Target plus smart Pool (Dharmacon; L-020429-02-0005)) in luminal breast cancer cell line MCF-7. The achieved knockdown of RHBG was determined 72 hours after transfection by qRT-PCR and was about 70% (FIG. 4A). RHBG and ACTB primers and probes used for qRT-PCR were the same as for Example 1 (Table 1).

FIGS. 4B and 4C show that inhibition of human RHBG by use of siRNA inhibited the proliferation of luminal breast cancer cells, and this was particularly noticeable 5 days after transfection with the siRNA directed against human RHBG (each row shows four identically treated wells, t-test was used and the data was presented as mean+/−SEM, P value after 3 days=0.0049, P value after 5 days<0.0001).

TABLE 3
Target sequences of siRNA against RHBG.

| siRNA | Sequence |
|---|---|
| Target Sequence 1 | UGGUCUUCCUGCAGCGUUA |
| | (SEQ ID NO: 16) |
| Target Sequence 2 | AUUCAGACCUCUUCGCCAU |
| | (SEQ ID NO: 17) |
| Target Sequence 3 | CCACAGUAACGCGGACAAU |
| | (SEQ ID NO: 18) |
| Target Sequence 4 | GGCCGUAACUGGGUACAAU |
| | (SEQ ID NO: 19) |

1. A method of treating a proliferative disease in a subject, comprising:
detecting at least one ammonium transporter in a tissue sample from the subject; and
administering a proliferative disease treatment to the subject.

2. The method according to claim 1, wherein the at least one ammonium transporter belongs to the Rhesus (Rh) protein family.

3. The method according to claim 1, wherein said at least one ammonium transporter is selected from the group consisting of Rh family, B glycoprotein (RHBG), Rh family, A glycoprotein (RHAG), Rh family, C glycoprotein (RHCG), aquaporin-3 (AQP3), aquaporin-7 (AQP7), aquaporin-8 (AQP8), aquaporin-9 (AQP9), aquaporin-10 (AQP10), Na⁺/K⁺ ATPase, Na⁺—K⁺-2Cl⁻ cotransporter (NKCC1), Na⁺/H⁺ exchanger NHE2, Na⁺/H⁺ exchanger NHE3, and combinations thereof; preferably wherein said at least one ammonium transporter is selected from the group consisting of RHBG, RHAG, RHCG, and combinations thereof; more preferably wherein said at least one ammonium transporter is RHBG.

4. The method according to claim 1, comprising the step of comparing the quantity or expression level of said at least one ammonium transporter in said sample with the reference quantity or expression level of said at least one ammonium transporter.

5. The method according to claim 1 comprising:
determining the quantity or expression level of said at least one ammonium transporter in a tissue sample from the subject;
comparing the determined quantity or expression level of said at least one ammonium transporter with a reference value, said reference value representing a known diagnosis, prediction and/or prognosis of said proliferative disease;
finding a deviation or no deviation of the determined quantity or expression level of said at least one ammonium transporter from said reference value;
attributing said finding of deviation or no deviation to a particular diagnosis, prediction, or prognosis of the proliferative disease in the subject.

6. The method according to claim 5, wherein an elevated quantity or expression level of said at least one ammonium transporter in the tissue sample from the subject as compared to the reference value allows for the diagnosis, prediction, or prognosis of the proliferative disease in the subject.

7. The method according to claim 5, wherein said reference value is determined from a tissue not affected by the proliferative disease, such as wherein said reference value is determined from healthy tissue.

8. A kit for diagnosing, predicting, prognosing and/or monitoring a proliferative disease in a subject, the kit comprising:
means for measuring the quantity or expression level of at least one ammonium transporter in a tissue sample from a subject; and
a reference value of the quantity or expression level of said at least one ammonium transporter or means for establishing said reference value, wherein said reference value represents a known diagnosis, prediction and/or prognosis of the proliferative disease, such as wherein said reference value corresponds to the quantity or expression level of said at least one ammonium transporter in a tissue not affected by the proliferative disease, such as in a healthy tissue, or wherein said reference value corresponds to the quantity or expression level of said at least one ammonium transporter in a tissue affected by the proliferative disease.

9. The kit according to claim 8, wherein the at least one ammonium transporter belongs to the Rh family, preferably wherein said at least one ammonium transporter is selected from the group consisting of RHBG, RHAG, RHCG, and combinations thereof.

10. A method of treating a proliferative disease in a subject, comprising administering to said subject a therapeutic or prophylactic agent capable of modulating, such as inhibiting or increasing, expression or activity of at least one ammonium transporter.

11. The method according to claim 10, wherein the at least one ammonium transporter belongs to the Rh family, preferably wherein said at least one ammonium transporter is selected from the group consisting of RHBG, RHAG, RHCG, and combinations thereof.

12. The method according to claim 10, wherein:
the agent modulates, such as interferes with or increases, ammonia and/or ammonium transport across the cell membrane mediated by said at least one ammonium transporter; and/or
the agent is capable of interacting with the gene or RNA, preferably mRNA, encoding said at least one ammonium transporter, or the agent is capable of interacting with said at least one ammonium transporter protein, or wherein said agent is capable of interacting with a naturally-occurring binding and/or regulatory partner of said at least one ammonium transporter, or with a gene or RNA, preferably mRNA, encoding said partner; and/or
said therapeutic or prophylactic agent is selected from the group consisting of a protein, a polypeptide, a peptide, a peptidomimetic, a nucleic acid, an aptamer, a small organic molecule, and a compound or combination of any two or more thereof; preferably wherein said agent is a gene-editing system, an antisense agent, an RNAi agent, such as siRNA or shRNA, or an antibody or functional fragment thereof, or a soluble receptor.

13. The method according to claim 1, wherein:
said proliferative disease is selected from the group consisting of hepatocellular carcinoma, hepatoma, hepatic carcinoma, liver cancer, colorectal cancer, colon cancer or carcinoma, and rectal cancer;
said proliferative disease is selected from the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, endometrial cancer, uterine carcinoma, salivary gland carcinoma, vulval cancer, thyroid cancer, anal carcinoma, penile carcinoma, head and neck cancer, adrenal gland cancer, paraganglioma, oesophageal cancer or carcinoma, kidney or renal cancer or carcinoma, urothelial cancer, bile duct cancer, prostate cancer, brain cancer, leukemia, and multiple myeloma; or
said proliferative disease is characterized by (over)expression of said at least one ammonium transporter.

14. (canceled)

15. (canceled)

16. The method according to claim 1, wherein said proliferative disease is luminal breast cancer.

17. An antibody or a functional fragment thereof, characterised in that the antibody or functional fragment thereof binds the epitope DSPPRLPALRGPSS of human RHBG as set out in SEQ ID NO: 15.

18. The kit according to claim 8, wherein:
said proliferative disease is selected from the group consisting of hepatocellular carcinoma, hepatoma, hepatic carcinoma, liver cancer, colorectal cancer, colon cancer or carcinoma, and rectal cancer;
said proliferative disease is selected from the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, endometrial cancer, uterine carcinoma, salivary gland carcinoma, vulval cancer, thyroid cancer, anal carcinoma, penile carcinoma, head and neck cancer, adrenal gland cancer, paraganglioma, oesophageal cancer or carcinoma, kidney or renal cancer or carcinoma, urothelial cancer, bile duct cancer, prostate cancer, brain cancer, leukemia, and multiple myeloma; or
said proliferative disease is characterised by (over)expression of said at least one ammonium transporter.

19. The kit according to claim 8, wherein said proliferative disease is luminal breast cancer.

20. The kit according to claim 8, wherein said proliferative disease is luminal breast cancer; said tissue sample is obtained from a breast tumour; and said reference value represents the quantity or expression level of said at least one ammonium transporter in a healthy breast tissue or in a basal breast cancer tissue.

21. The method according to claim 10, wherein:
said proliferative disease is selected form the group consisting of hepatocellular carcinoma, hepatoma, hepatic carcinoma, liver cancer, colorectal cancer, colon cancer or carcinoma, and rectal cancer;
said proliferative disease is selected form the group consisting of breast cancer, squamous cell cancer, lung cancer, cancer of the peritoneum, gastric cancer, stomach cancer, pancreatic cancer, glioma, glioblastoma, skin cancer, uterus cancer, cervical cancer, ovarian cancer, bladder cancer, endometrial cancer, uterine carcinoma, salivary gland carcinoma, vulval cancer, thyroid cancer, anal carcinoma, penile carcinoma, head and neck cancer, adrenal gland cancer, paraganglioma, oesophageal cancer or carcinoma, kidney or renal cancer or carcinoma, urothelial cancer, bile duct cancer, prostate cancer, brain cancer, leukemia, and multiple myeloma; or
said proliferative disease is characterised by (over)expression of said at least one ammonium transporter.

22. The method according to claim 10, wherein said proliferative disease is luminal breast cancer.

23. A method of diagnosing a proliferative disease in a subject, said method comprising detecting whether at least one ammonium transporter is present in a tissue sample from the subject by contacting the tissue sample with an antibody or a functional fragment thereof that binds the epitope DSPPRLPALRGPSS of human RHBG as set out in SEQ ID NO: 15 and detecting binding between the at least one ammonium transporter and the antibody or the functional fragment thereof; and diagnosing the subject with a proliferative disease when the presence of at least one ammonium transporter is detected in the tissue sample.


SEQUENCE LISTING (21 sequences)

SEQ ID NO: 1 - DNA, 1349 bp, Homo sapiens
atggccgggtctcctagccgcgccgcgggccggcgactgcagcttcccctgctgtgcctcttcctccagggcgccactgccgtcctctttgctgtctttgtccgctacaaccacaaaaccgacgctgccctctggcaccggagcaaccacagtaacgcggacaatgaattttactttcgctacccaagcttccaggacgtgcatgccatggtcttcgtgggctttggcttcctcatggtcttcctgcagcgttacggcttcagcagcgtgggcttcaccttcctcctggccgcctttgccctgcagtggtccacactggtccagggctttctccactccttccacggtggccacatccatgttggcgtggagagcatgatcaatgctgacttttgtgcgggggccgtgctcatctcctttggtgccgtcctgggcaagaccgggcctacccagctgctgctcatggccctgctggaggtggtgctgtttggcatcaatgagtttgtgctccttcatctcctgggggtgagagatgccggaggctccatgactatccacacctttggtgcctacttcgggctcgtcctttcgcgggttctgtacaggccccagctggagaagagcaagcaccgccagggctccgtctaccattcagacctcttcgccatgattgggaccatcttcctgtggatcttctggcctagcttcaatgctgcactcacagcgctgggggctgggcagcatcggacggccctcaacacatactactccctggctgccagcacccttggcacctttgccttgtcagcccttgtaggggaagatgggaggcttgacatggtccacatccaaaatgcagcgctggctggaggggttgtggtggggacctcaagtgaaatgatgctgacaccctttggggctctggcagctggcttcttggctgggactgtctccacgctggggtacaagttcttcacgcccatccttgaatcaaaattcaaagtccaagacacatgtggagtccacaacctccatgggatgccgggggtcctgggggccctcctgggggtccttgtggctggacttgccacccatgaagcttacggagatggcctggagagtgtgtttccactcatagccgagggccagcgcagtgccacgtcacaggccatgcaccagctcttcgggctgtttgtcacactgatgtttgcctctgtgggcgggggccttggagggctcctgctgaagctaccctttctggactccccccccagactcccagcactacgaggaccaagttcactggcaggtgcctggcgagcatgaggataaagcccagagacctctga

SEQ ID NO: 2 - protein, 441 aa, Homo sapiens
MAGSPSRAAGRRLQLPLLCLFLQGATAVLFAVFVRYNHKTDAALWHRSNHSNADNEFYFRYPSFQDVHAMVFVGFGFLMVFLQRYGFSSVGFTFLLAAFALQWSTLVQGFLHSFHGGHIHVGVESMINADFCAGAVLISFGAVLGKTGPTQLLLMALLEVVLFGINEFVLLHLLGVRDAGGSMTIHTFGAYFGLVLSRVLYRPQLEKSKHRQGSVYHSDLFAMIGTIFLWIFWPSFNAALTALGAGQHRTALNTYYSLAASTLGTFALSALVGEDGRLDMVHIQNAALAGGVVVGTSSEMMLTPFGALAAGFLAGTVSTLGYKFFTPILESKFKVQDTCGVHNLHGMPGVLGALLGVLVAGLATHEAYGDGLESVFPLIAEGQRSATSQAMHQLFGLFVTLMFASVGGGLGGLLLKLPFLDSPPRLPALRGPSSLAGAWRA

SEQ ID NO: 3 - DNA, 20 bp, artificial
  forward RHBG primer
ctgtgagtgcagcattgaag

SEQ ID NO: 4 - DNA, 21 bp, artificial
  reverse RHBG primer
ctaccattcagacctcttcgc

SEQ ID NO: 5 - DNA, 25 bp, artificial
  RHBG probe
  misc_feature (1)..(1): 6-FAM fluorescent dye
  misc_feature (9)..(10): ZEN? internal quencher is inserted between nucleotide positions 9 and 10
  misc_feature (25)..(25): Iowa Black?-quencher
ccatcttcctgtggatcttctggcc

SEQ ID NO: 6 - DNA, 21 bp, artificial
  forward ACTB primer
aagtcagtgtacaggtaagcc

SEQ ID NO: 7 - DNA, 22 bp, artificial
  reverse ACTB primer
gtcccccaacttgagatgtatg

SEQ ID NO: 8 - DNA, 19 bp, artificial
  ACTB probe
  misc_feature (1)..(1): 6-FAM fluorescent dye
  misc_feature (9)..(10): ZEN? internal quencher is inserted between nucleotide positions 9 and 10
  misc_feature (19)..(19): Iowa Black?-quencher
ctgcctccacccactccca

SEQ ID NO: 9 - DNA, 20 bp, artificial
  forward RHBG primer
cctcaagtgaaatgatgctg

SEQ ID NO: 10 - DNA, 20 bp, artificial
  reverse RHBG primer
attttgattcaaggatgggc

SEQ ID NO: 11 - DNA, 20 bp, artificial
  forward ACTB primer
ctggaacggtgaaggtgaca

SEQ ID NO: 12 - DNA, 23 bp, artificial
  reverse ACTB primer
aagggacttcctgtaacaatgca

SEQ ID NO: 13 - DNA, 20 bp, artificial
  forward POLR2A primer
catcatcatccatcttgtcc

SEQ ID NO: 14 - DNA, 20 bp, artificial
  reverse POLR2A primer
aagatcaatgctggttttgg

SEQ ID NO: 15 - protein, 14 aa, Homo sapiens
DSPPRLPALRGPSS

SEQ ID NO: 16 - RNA, 19 nt, Homo sapiens
uggucuuccugcagcguua

SEQ ID NO: 17 - RNA, 19 nt, Homo sapiens
auucagaccucuucgccau

SEQ ID NO: 18 - RNA, 19 nt, Homo sapiens
ccacaguaacgcggacaau

SEQ ID NO: 19 - RNA, 19 nt, Homo sapiens
ggccguaacuggguacaau

SEQ ID NO: 20 - protein, 409 aa, Homo sapiens
MRFTFPLMAIVLEIAMIVLFGLFVEYETDQTVLEQLNITKPTDMGIFFELYPLFQDVHVMIFVGFGFLMTFLKKYGFSSVGINLLVAALGLQWGTIVQGILQSQGQKFNIGIKNMINADFSAATVLISFGAVLGKTSPTQMLIMTILEIVFFAHNEYLVSEIFKASDIGASMTIHAFGAYFGLAVAGILYRSGLRKGHENEESAYYSDLFAMIGTLFLWMFWPSFNSAIAEPGDKQCRAIVNTYFSLAACVLTAFAFSSLVEHRGKLNMVHIQNATLAGGVAVGTCADMAIHPFGSMIIGSIAGMVSVLGYKFLTPLFTTKLRIHDTCGVHNLHGLPGVVGGLAGIVAVAMGASNTSMAMQAAALGSSIGTAVVGGLMTGLILKLPLWGQPSDQNCYDDSVYWKVPKTR

SEQ ID NO: 21 - protein, 479 aa, Homo sapiens
MAWNTNLRWRLPLTCLLLQVIMVILFGVFVRYDFEADAHWWSERTHKNLSDMENEFYYRYPSFQDVHVMVFVGFGFLMTFLQRYGFSAVGFNFLLAAFGIQWALLMQGWFHFLQDRYIVVGVENLINADFCVASVCVAFGAVLGKVSPIQLLIMTFFQVTLFAVNEFILLNLLKVKDAGGSMTIHTFGAYFGLTVTRILYRRNLEQSKERQNSVYQSDLFAMIGTLFLWMYWPSFNSAISYHGDSQHRAAINTYCSLAACVLTSVAISSALHKKGKLDMVHIQNATLAGGVAVGTAAEMMLMPYGALIIGFVCGIISTLGFVYLTPFLESRLHIQDTCGINNLHGIPGIIGGIVGAVTAASASLEVYGKEGLVHSFDFQGFNGDWTARTQGKFQIYGLLVTLAMALMGGIIVGLILRLPFWGQPSDENCFEDAVYWEMPEGNSTVYIPEDPTFKPSGPSVPSVPMVSPLPMASSVPLVP